(19) United States
(12) Patent Application Publication
(10) Pub. No.: US 2007/0275404 A1
(43) Pub. Date: Nov. 29, 2007

(54) Gene expression signatures associated with tumor stromal cells

(76) Inventors: Jan Matthijs van de Rijn, La Honda, CA (US); Robert West, Stanford, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/801,459

(22) Filed: May 9, 2007

Related U.S. Application Data
(60) Provisional application No. 60/799,561, filed on May 10, 2006.

(51) Int. Cl. C12Q 1/68; G06F 19/00

(52) U.S. Cl. 435/6; 702/20

(57) ABSTRACT

Methods are provided for classification of solid tumors other than soft tissue tumors; e.g. carcinomas. The tumor mass of such cancers comprises neoplastic cells of epithelial origin, and surrounding stroma. Methods are provided for classification and analysis of such tumors based on the gene expression signature of the tumor stromal cell component.

This invention was made with Government support under contract DAMD17-03-1 awarded by the Department of Defense and this invention was made with Government support under contract RCA112270 awarded by the National Institutes of Health. The Government has certain rights in this invention.

In recent years, microarray analysis of gene expression patterns has provided a way to improve the diagnosis and risk stratification of many cancers, as well identifying candidate genes for therapeutic intervention. Unsupervised analysis of global gene expression patterns may identify molecularly distinct subtypes of cancer or of cells within tumors, distinguished by extensive differences in gene expression. Such molecular subtypes can be associated with different clinical outcomes. Global gene expression pattern can also be examined for features that correlate with clinical behavior to create prognostic signatures.

Identification of differentially expressed gene products also furthers the understanding of the progression and nature of complex diseases such as cancer, and is key to identifying the genetic factors that are responsible for the phenotypes associated with development of, for example, the metastatic phenotype. Identification of gene products that are differentially expressed at various stages, and in various types of cancers, can both provide for early diagnostic tests, and further serve as therapeutic targets. Additionally, the product of a differentially expressed gene can be the basis for screening assays to identify chemotherapeutic agents that modulate its activity (e.g. its expression, biological activity, and the like).

By detailing the expression level of thousands of genes simultaneously in tumor cells or their surrounding stroma, gene expression profiles of tumors can provide “molecular portraits” of human cancers. The variations in gene expression patterns in human cancers are multidimensional and typically represent the contributions and interactions of numerous distinct cells and diverse physiological, regulatory, and genetic factors. Although gene expression patterns that correlate with different clinical outcomes can be identified from microarray data, the biological processes that the genes represent and thus the appropriate therapeutic interventions are generally not obvious.

In recent years scientists have determined multiple factors that affect transformed cells in the body—that a cell becomes malignant as a result of changes to its genetic material and that accompanying biological characteristics of the cell also change. These changes are unique molecular “signatures” and serve as signals of the presence of cancer. However, the neoplastic cancer cell is only part of the story in cancer development. As a cancer cell grows within the architecture of the body's tissues and organs, it interacts with its surrounding environment.

Mounting evidence now suggests that a dynamic interaction occurs between the cancer cell and its microenvironment, with each profoundly influencing the behavior of the other. This “tumor microenvironment,” is populated with a variety of different cell types, is rich in growth factors and enzymes, and includes parts of the blood and lymphatic systems. It promotes some of the most destructive characteristics of cancer cells and permits the tumor to grow and spread.

Although the cells in the microenvironment may not be genetically altered, their behavior can be changed through interactions with tumor cells. The tumor cells and their surrounding environment both need to be fully characterized in order to understand how cancer grows in the body, and both need to be considered when developing new interventions to fight disease: Evidence suggests that the interaction between cancer cells and their microenvironment is key to this transition from transformed cell to a tumor mass. It has been observed that the influence between the environment and tumor cells is bidirectional. Non-cancerous cells that adjoin a cancerous tumor often take on atypical characteristics and exert a profound influence on a cancer cell's ability to develop into a tumor.

It is becoming evident that events outside the cancer cell are as important to disease development as the disrupted processes inside the cell. This broadened concept of cancer requires an understanding of stromal cells, and the interplay between the cancer cell and its immediate environment. This new perspective may also open new avenues to treatment. Rather than targeting the cancer cell alone, new treatment approaches can potentially target the features of the microenvironment that allow tumors to develop and progress. In addition, because the microenvironment often exerts considerable influence over tumor cells in the early stages of tumor development; it promises to be an attractive target for prevention efforts. The present invention addresses this issue.

### SUMMARY OF THE INVENTION

Methods are provided for classification of solid tumors other than soft tissue tumors; e.g. carcinomas. The tumor mass of such cancers comprises neoplastic cells of epithelial origin, and surrounding stroma. Methods are provided for classification and analysis of such tumors based on the gene expression signature of the tumor stromal cell component.

In the methods of the invention, reference signatures for a tumor stromal cell component are derived from the gene expression profiles of soft tissue tumors, e.g. sarcomas. Such soft tissue gene expression sets (STS) comprise information of the genes that are specifically expressed in certain types of soft tissue cells; and provide insight into the nature of the tumor stromal cell component. The gene expression sets further provide targets for therapeutic intervention in the treatment of carcinomas.

It is shown herein that varied carcinomas have a commonality in stromal cell components, even where there is not a commonality in the neoplastic epithelial cell component. This stromal cell component allows for classification and treatment of carcinomas regardless of the origin of the neoplastic cells. Classification according to STS signature allows optimization of treatment, and determination of whether on whether to proceed with a specific therapy, and how to optimize dose, choice of treatment, and the like.

For the methods of the invention, a gene expression profile is utilized from one or more, usually two or more soft tissue tumors. Tumors of interest include, without limitation, Evan's tumor; nodular fasciitis; desmoid-type fibromatosis; solitary fibrous tumor; dermatofibrosarcoma protuberans (DFSP); angiosarcoma; epithelioid hemangioendothelioma; tenosynovial giant cell tumor (TGCT); pigmented villonodular synovitis (PVNS); fibrous dysplasia; myxofibrosarcoma; fibrosarcoma; synovial sarcoma; malignant peripheral nerve sheath tumor; neurofibroma; and pleomorphic adenoma of soft tissue. A gene expression dataset from a soft tissue tumor is compared to a gene expression dataset from a second soft tissue tumor, e.g. using one or more of the tumor profiles provided herein. Genes that are common to both tumors are withdrawn from the dataset, leaving the dataset of unique genes (i.e. unique with respect to another soft tissue tumor). The dataset of unique genes is useful in classification of carcinomas; as a source of probes for in situ hybridization; as a platform for discovery of therapeutic targets; and the like.

In some embodiments of the invention, a set of unique sequences from a soft tissue tumor are used as source of probes for in situ hybridization of solid cancers other than soft tissue cancers, e.g. for the in situ hybridization of carcinomas. In such methods, the set of uniquely expressed sequence is analyzed for a high level of differential expression in the soft tissue tumor; and a high level of absolute expression of the mRNA. Sequences having these characteristics are selected, and the sequence used to provide a probe. Probes are labeled, e.g. with a fluorescent label, and hybridized to tissue sections of non-soft tissue tumors, e.g. carcinomas. The staining is used to identify and classify features of stromal cells within the tumor. In some embodiments, probes are useful for characterization of multiple carcinomas, e.g. two or more of breast carcinoma, lung carcinoma, colorectal carcinoma; prostate carcinoma; ovarian carcinoma, etc.

In other embodiments, a set of unique genes from a soft tissue tumor is used as a platform for identifying targets useful in therapy of solid tumors other than soft tissue tumors, e.g. carcinomas. Sequences within the STS are analyzed for specific features of interest, including expression on the cell surface; presence of protein kinase or protein phosphatase domains; transmembrane regions; and the like. Sequences having these characteristics are selected, and the sequence used to identify therapeutic agents. In some instances, candidate target sequences will also be useful as in situ hybridization probes. In other examples, agents are initially screened for the ability to bind to a candidate target; and will undergo a secondary screening for activity against a carcinoma in a model that provides a stromal component; e.g. xenotransplant; animal models; tissue sections; etc.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Soft Tissue Tumor Gene Expression. Unsupervised hierarchical clustering of ten cases of DTF (blue), 13 cases of SFT (orange), and 35 other previously examined soft tissue tumors (black) based on expression profiling on 42,000-element cDNA microarrays. Red represents high expression, black represents median expression, green represents low expression, and grey represents no data. DFSP, dermatofibrosarcoma protuberans; GIST, gastrointestinal stromal tumor; LMS, leiomyosarcomas, MFH, malignant fibrous histiocytomas; SS, synovial sarcoma.

FIG. 2. Localization of Fibroblastic Gene Expression. Comparison of expression of two SFT markers APOD (ISH) and CD34 (IHC), and two DTF markers CTHRC1 (ISH) and OSF2 (ISH) in SFT and DTF. SFTs express ApoD and CD34 whereas DTFs express CTHRC1 and OSF2. H&E, hematoxylin-eosin. Magnification=600×.

FIG. 3. Fibroblastic Markers in Non-Neoplastic Tissue. (A) Skin adnexa, (B) breast, (C) dermis, (D) reactive, and (E) keloid tissue arranged in rows. Fibroblastic markers: CD34 (IHC), APOD (ISH), CTHRC1 (ISH) and OSF2 (ISH) arranged in columns. SFTs express APOD and CD34 whereas DTFs express CTHRC1 and OSF2. Magnification=600×.

FIGS. 4A-4B. Fibroblast Markers in Breast Carcinoma (A) Examples of SFT (APOD [ISH] and CD34) and DTF (CTHRC1 [ISH] and OSF2 [ISH]) expression in breast carcinoma stroma. Each panel shows expression of the marker that is restricted to the fibroblasts between neoplastic cells. Magnification=600×. (B) Hierarchical clustering of 24 breast carcinomas based on TMA staining with fibroblast markers: CD34 (IHC), APOD (ISH), CTHRC1 (ISH), and OSF2 (ISH). Bright red represents high expression, dull red represents intermediate high expression, green represents negative expression, and white represents no data. The DTF-associated cluster is highlighted in blue. The SFT-associated cluster is highlighted in orange. Most breast carcinomas express either a DTF or SFT gene in the stromal fibroblasts.

FIG. 5. Hierarchical Clustering of 295 Breast Carcinomas with 471 SFT and DTF Genes. Within the heatmap, red represents high expression, black represents median expression, and green represents low expression. Sidebar on right indicates which tumor the gene is positively associated with: pink is SFT and purple is DTF. Sidebar on left indicates gene cluster.

FIG. 6. Outcome Data. Statistical method of the y-axis is Kaplan-Meier survival curves compared by the Cox-Mantel log-rank test. The x-axis unit of measure is years. (A) Time to first recurrence for tumor group A versus all other tumors. (B) Time to first recurrence for tumor group B versus all other tumors. (C) Survival outcome for tumor group A versus all others. (D) Survival outcome for tumor group B versus all others.

### DETAILED DESCRIPTION OF THE EMBODIMENTS

Methods are provided for classification of solid tumors other than soft tissue tumors; e.g. carcinomas based on the gene expression signature of the tumor stromal cell component. In the methods of the invention, reference signatures for the tumor stromal cell component are derived from the genetic profiles of soft tissue tumors, e.g. sarcomas.

Typically a gene expression profile is utilized from one or more, usually two or more soft tissue tumors. A gene expression dataset from a soft tissue tumor is compared to a gene expression dataset from a second soft tissue tumor, e.g. using one or more of the tumor profiles provided herein. The dataset of unique expressed genes (i.e. unique with respect to another soft tissue tumor) is useful in classification of carcinomas; as a source of probes for in situ hybridization; as a platform for discovery of therapeutic targets; and the like. In certain embodiments, the expression profile is determined using a microarray. In other embodiments the expression profile is determined by quantitative PCR or other quantitative methods for measuring mRNA.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims. In this specification and the appended claims, the singular forms “a,” “an” and “the” include plural reference unless the context clearly dictates otherwise.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the subject components of the invention that are described in the publications, which components might be used in connection with the presently described invention.

As summarized above, the subject invention is directed to methods of classification of cancers, as well as reagents and kits for use in practicing the subject methods. The methods may also determine an appropriate level of treatment for a particular cancer.

Methods are also provided for optimizing therapy, by first classification, and based on that information, selecting the appropriate therapy, dose, treatment modality, etc. which optimizes the differential between delivery of an anti-proliferative treatment to the undesirable target cells, while minimizing undesirable toxicity. The treatment is optimized by selection for a treatment that minimizes undesirable toxicity, while providing for effective anti-proliferative activity.

Applicants herein specifically incorporate by reference the teachings and associated published information of each of West et al. (2005) PLoS 3(6)e187; of Van de Rijn et al. (2006) Annu. Rev. Pathol. Mech. 1:435-466; of West and Ven de Rijn (2006) Histopathology 48:22-31; of West et al. (2006) Proc Natl Acad Sci U S A 103(3):690-5; and of Subramanian et al. (2005) J Pathol. 206(4):433-44.

Soft tissue tumors (STT) are a highly diverse group of rare tumors that are derived from connective tissue. More than 100 different malignant and benign soft tissue neoplasms can be recognized by histologic examination. Few diagnostic markers are known.

Tumors of connective tissue include alveolar soft part sarcoma; angiomatoid fibrous histiocytoma; chondromyoxid fibroma; skeletal chondrosarcoma; extraskeletal myxoid chondrosarcoma; clear cell sarcoma; desmoplastic small round-cell tumor; dermatofibrosarcoma protuberans; endometrial stromal tumor; Ewing's sarcoma; fibromatosis (Desmoid); fibrosarcoma, infantile; gastrointestinal stromal tumor; bone giant cell tumor; tenosynovial giant cell tumor; inflammatory myofibroblastic tumor; uterine leiomyoma; leiomyosarcoma; lipoblastoma; typical lipoma; spindle cell or pleomorphic lipoma; atypical lipoma; chondroid lipoma; well-differentiated liposarcoma; myxoid/round cell liposarcoma; pleomorphic liposarcoma; myxoid malignant fibrous histiocytoma; high-grade malignant fibrous histiocytoma; myxofibrosarcoma; malignant peripheral nerve sheath tumor; mesothelioma; neuroblastoma; osteochondroma; osteosarcoma; primitive neuroectodermal tumor; alveolar rhabdomyosarcoma; embryonal rhabdomyosarcoma; benign or malignant schwannoma; synovial sarcoma; Evan's tumor; nodular fasciitis; desmoid-type fibromatosis; solitary fibrous tumor; dermatofibrosarcoma protuberans (DFSP); angiosarcoma; epithelioid hemangioendothelioma; tenosynovial giant cell tumor (TGCT); pigmented villonodular synovitis (PVNS); fibrous dysplasia; myxofibrosarcoma; fibrosarcoma; synovial sarcoma; malignant peripheral nerve sheath tumor; neurofibroma; and pleomorphic adenoma of soft tissue.

Including in the designation of soft tissue tumors are neoplasias derived from fibroblasts, myofibroblasts, histiocytes, vascular cells/endothelial cells and nerve sheath cells. All of these cells have representation in stroma of epithelial neoplasms such as breast carcinoma and colon carcinoma.

The invention finds use in the prevention, treatment, detection or research into solid cancers other than those of soft tissue, particularly carcinomas. Carcinomas are malignancies that originate in the epithelial tissues. Epithelial cells cover the external surface of the body, line the internal cavities, and form the lining of glandular tissues. In adults, carcinomas are the most common forms of cancer. Carcinomas include the a variety of adenocarcinomas, for example in prostate, lung, etc.; adernocartical carcinoma; hepatocellular carcinoma; renal cell carcinoma, ovarian carcinoma, carcinoma in situ, ductal carcinoma, carcinoma of the breast, basal cell carcinoma; squamous cell carcinoma; transitional cell carcinoma; colon carcinoma; nasopharyngeal carcinoma; multilocular cystic renal cell carcinoma; oat cell carcinoma, large cell lung carcinoma; small cell lung carcinoma; etc. Carcinomas may be found in prostrate, pancreas, colon, brain (usually as secondary metastases), lung, breast, skin, etc.

“Diagnosis” as used herein generally includes determination of a subject's susceptibility to a disease or disorder, determination as to whether a subject is presently affected by a disease or disorder, prognosis of a subject affected by a disease or disorder (e.g., identification of pre-metastatic or metastatic cancerous states, stages of cancer, or responsiveness of cancer to therapy), and use of therametrics (e.g., monitoring a subject's condition to provide information as to the effect or efficacy of therapy).

The term “biological sample” encompasses a variety of sample types obtained from an organism and can be used in a diagnostic or monitoring assay. The term encompasses blood and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain-components. The term encompasses a clinical sample, and also includes cells in cell culture, cell supernatants, cell lysates, serum, plasma, biological fluids, and tissue samples.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the, disease symptom, i.e., arresting its development; or (c) relieving the disease symptom, i.e., causing regression of the disease or symptom.

The terms “individual,” “subject,” “host,” and “patient,” used interchangeably herein and refer to any mammalian subject for whom diagnosis, treatment, or therapy is desired, particularly humans. Other subjects may include cattle, dogs, cats, guinea pigs, rabbits, rats, mice, horses, and the like.

A “host cell”, as used herein, refers to a microorganism or a eukaryotic cell or cell line cultured as a unicellular entity which can be, or has been, used as a recipient for a recombinant vector or other transfer polynucleotides, and include the progeny of the original cell which has been transfected. It is understood that the progeny of a single cell may not necessarily be completely identical in morphology or in genomic or total DNA complement as the original parent, due to natural, accidental, or deliberate mutation.

The term “normal” as used in the context of “normal cell,” is meant to refer to a cell of an untransformed phenotype or exhibiting a morphology of a non-transformed cell of the tissue type being examined.

“Cancerous phenotype” generally refers to any of a variety of biological phenomena that are characteristic of a cancerous cell, which phenomena can vary with the type of cancer. The cancerous phenotype is generally identified by abnormalities in, for example, cell growth or proliferation (e.g., uncontrolled growth or proliferation), regulation of the cell cycle, cell mobility, cell-cell interaction, or metastasis, etc.

“Therapeutic target” generally refers to a gene or gene product that, upon modulation of its activity (e.g., by modulation Of expression, biological activity, and the like), can provide for modulation of the cancerous phenotype.

As used throughout, “modulation” is meant to refer to an increase or a decrease in the indicated phenomenon (e.g., modulation of a biological activity refers to an increase in a biological activity or a decrease in a biological activity).

In the methods of the invention, reference signatures for a tumor stromal cell component are derived from the gene expression profiles of soft tissue tumors as described above. A “STS signature” is a dataset that has been obtained from a soft tissue tumor, and provides information on the set of genes particular to that soft tissue cell type, e.g. fibroblast type; endothelial cell type, etc. A useful signature may be obtained from all or a part of the gene dataset, usually the signature will comprise information from at least about 20 genes, more usually at least about 30 genes, at least about 35 genes, at least about 45 genes, at least about 50 genes, or more, up to the complete dataset. Where a subset of the dataset is used, the subset may comprise upregulated genes, downregulated genes, or a combination thereof.

The dataset is obtained by various means known in the art, e.g. by hybridization of mRNA or a polynucleotide derived therefrom to an array. Preferably expression profiling utilizes two or more, three or more, four or more different tumors from a particular soft tissue tumor; and will usually include expression data from one or more unrelated soft tissue tumors for filtering purposes.

For example, analysis of solitary fibrous tumors (SFT) may comprise data from 2, 3, 4, 5 or more different tumors within this classification. The raw data for hybridization to an array is then filtered. Suitable criteria for filtering include achieving a pre-determined ratio of hybridization intensity to background intensity, e.g. a ratio of at least about 1.5, at least about 2.0, at least about 2.5 versus background intensity.

Data may be further filtered for absolute level of expression, relative to the mean expression level within the tumor classification, and optionally as compared to other samples. A cut-off for expression level may be at least about three-fold greater relative to the mean expression; at least about four-fold; at least about five-fold, or more. A further selection may be made for genes that had at least about 70%, at least about 80%, at least about 90% or more measurable data across the set of tumors being analyzed.

The filtered data is the grouped, for example using unsupervised hierarchical clustering, across data from unrelated soft tissue tumors. For example, data from SFT tumors may be clustered with expression data from one or more of desmoid-type fibromatosis; extraskeletal myxoid chondrosarcoma; Evan's tumor; nodular fasciitis; dermatofibrosarcoma protuberans (DFSP); angiosarcoma; epithelioid hemangioendothelioma; tenosynovial giant cell tumor (TGCT); pigmented villonodular synovitis (PVNS); fibrous dysplasia; myxofibrosarcoma; fibrosarcoma; synovial sarcoma; malignant peripheral nerve sheath tumor; neurofibroma; pleomorphic adenoma of soft tissue; and the like. The analysis clusters expressed genes into groups with similar expression patterns across the tumors tested and clusters the tumor specimens based on their gene expression profile.

The analysis thus performed provides a set of highly expressed genes (STS signature) that distinguish the soft tissue tumor from other soft tissue tumors. In one embodiment of the invention, a reference STS profile is obtained from one or more of the soft tissue tumors selected from desmoid-type fibromatosis; extraskeletal myxoid chondrosarcoma; Evan's tumor; nodular fasciitis; dermatofibrosarcoma protuberans (DFSP); angiosarcoma; epithelioid hemangioendothelioma; tenosynovial giant cell tumor (TGCT); pigmented villonodular synovitis (PVNS); fibrous dysplasia; myxofibrosarcoma; fibrosarcoma; synovial sarcoma; malignant peripheral nerve sheath tumor; neurofibroma; or pleomorphic adenoma of soft tissue; which reference profile comprises information for genes that are clustered relative to at least one other soft tissue tumor.

Because of the generally clonal nature of such soft tissue tumors, the STS signature is also useful in the classification of the soft tissue component of carcinomas and other solid tissues. It is shown herein that varied carcinomas have a commonality in stromal cell components, even where there is not a commonality in the neoplastic epithelial cell component. This stromal cell component allows for classification and treatment of carcinomas regardless of the origin of the neoplastic cells. Classification according to STS signature allows optimization of treatment, and determination of whether on whether to proceed with a specific therapy, and how to optimize dose, choice of treatment, and the like.

For various methods, a subset of genes may be utilized, where the subset may comprise expression data for 5, 10, 20, 25, 50, 100 or more of, including all of the listed genes/proteins in a reference STS profile. Where the expression profile comprises a subset of genes, the subset may be selected for various criteria, including, without limitation, quantitative data, for example the 10% most highly expressed sequences; the 25% most highly expressed sequences; the 50% highly expressed sequences; the 75% highly expressed sequences. Alternatively, the subset may comprise genes that are most specific for the soft tissue type of interest.

Such a subset may be chosen for usefulness in in situ hybridization. Parameters for such selection may include specificity of expression, e.g. based on analysis with SAM (see Tibshirani et al., herein incorporated by reference) for the genes that have the most differential diagnostic capacity. A second criterion is for a relatively high level of messenger RNA in those tumors in which they react positively. For example, the arbitrary absolute level of expression of 9,000 or higher as indicated by the red channel fluorescence for the gene may be selected. Alternatively genes may be selected that at least a five-fold increased expression compared to the mean level of expression, at least abut 7.5-fold increased, at least about 10-fold increased, or more.

In another embodiment, a subset of sequences is selected based on function of the gene product. Such subsets may include genes involved in extracellular matrix functions, e.g. collagens, cadherins, and other structural proteins, matrix metalloproteases, growth factors in fibrotic response; and the like; genes involved in growth factor pathways, e.g. growth factors and growth factor receptors; genes involved in wnt signaling pathways, e.g. wnts, notched, β-catenin, frizzled, Dkk, etc.; genes involved in angiogenesis; and the like.

In other embodiments, the subset of genes is selected based on general characteristics of the encoded polypeptides. For example, a subset may comprise sequences comprising a transmembrane domain; sequences comprising a kinase domain; sequences comprising a phosphatase domain; sequences comprising a signal sequence, and the like. In other embodiments, a subset of sequences is selected for investigation as a therapeutic target, for example a set of genes in an STS signature may be filtered for level of expression, expression on the cell surface, specificity of expression relative to neoplastic epithelial cells, and for expression in the stromal cell component of varied carcinomas or other solid tumors.

The STS profiles of the invention are useful in categorizing expression profiles of test samples derived from carcinomas and other solid tumors comprising a stromal cell component. The test sample is classified according to its similarity to one or more STS profiles, where such profiles are associated with a clinical outcome. For example, association of breast or ovarian carcinoma profiles with SFT and DTF gene sets resulted in a clustering of carcinomas with the DTF profile, which had a statistically significant better overall survival and metastasis-free survival when compared to the rest of the dataset. In contrast, carcinoma profiles that clustered with SFT reference profile had a statistically significant worse overall survival and metastasis-free survival when compared to the rest of the dataset.

The term expression profile is used broadly to include a genomic expression profile, e.g., an expression profile of mRNAs, or a proteomic expression profile, e.g., an expression profile of one or more different proteins. Profiles may be generated by any convenient means for determining differential gene expression between two samples, e.g. quantitative hybridization of mRNA, labeled mRNA, amplified mRNA, cRNA, etc., quantitative PCR, ELISA for protein quantitation, and the like. A subject or patient tumor sample, e.g., cells or collections thereof, e.g., tissues, is assayed. Samples are collected by any convenient method, as known in the art. Additionally, tumor cells may be collected and tested to determine the relative effectiveness of a therapy in causing differential death between normal and diseased cells. Genes/proteins of interest are genes/proteins that are found to be predictive, including the genes/proteins provided above, where the expression profile may include expression data for 5, 10, 20, 25, 50, 100 or more of, including all of the listed genes/proteins.

In certain embodiments, the expression profile obtained is a genomic or nucleic acid expression profile, where the amount or level of one or more nucleic acids in the sample is determined. In these embodiments, the sample that is assayed to generate the expression profile employed in the diagnostic methods is one that is a nucleic acid sample. The nucleic acid sample includes a plurality or population of distinct nucleic acids that includes the expression information of the phenotype determinative genes of interest of the cell or tissue being diagnosed. The nucleic acid may include RNA or DNA nucleic acids, e.g., mRNA, cRNA, cDNA etc., so long as the sample retains the expression information of the host cell or tissue from which it is obtained.

The sample may be prepared in a number of different ways, as is known in the art, e.g., by mRNA isolation from a cell, where the isolated mRNA is used as is, amplified, employed to prepare cDNA, cRNA, etc., as is known in the differential expression art. The sample is typically prepared from a tumor cell or tissue harvested from a subject to be diagnosed, using standard protocols, where cell types or tissues from which such nucleic acids may be generated include any tissue in which the expression pattern of the to be determined phenotype exists. Cells may be cultured prior to analysis.

The expression profile may be generated from the initial nucleic acid sample using any convenient protocol. While a variety of different manners of generating expression profiles are known, such as those employed in the field of differential gene expression analysis, one representative and convenient type of protocol for generating expression profiles is array based gene expression profile generation protocols. Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively.

Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the phenotype determinative genes whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acid provides information regarding expression for each of the genes that have been probed, where the expression information is in terms of whether or not the gene is expressed and, typically, at what level, where the expression data, i.e., expression profile, may be both qualitative and quantitative.

Alternatively, non-array based methods for quantitating the levels of one or more nucleic acids in a sample may be employed, including quantitative PCR, and the like.

Where the expression profile is a protein expression profile, any convenient protein quantitation protocol may be employed, where the levels of one or more proteins in the assayed sample are determined. Representative methods include, but are not limited to; proteomic arrays, flow cytometry, standard immunoassays, etc.

Following obtainment of the expression profile from the sample being assayed, the expression profile is compared with a reference or control profile to make a diagnosis. A reference or control profile is provided, or may be obtained by empirical methods using the methods described herein. In certain embodiments, the obtained expression profile is compared to a single reference/control profile to obtain information regarding the phenotype of the cell/tissue being assayed. In yet other embodiments, the obtained expression profile is compared to two or more different reference/control profiles to obtain more in depth information regarding the phenotype of the assayed cell/tissue. For example, the obtained expression profile may be compared to a positive and negative reference profile to obtain confirmed information regarding whether the cell/tissue has the phenotype of interest.

The difference values, i.e. the difference in expression may be performed using any convenient methodology, where a variety of methodologies are known to those of skill in the array art, e.g., by comparing digital images of the expression profiles, by comparing databases of expression data, etc. Patents describing ways of comparing expression profiles include, but are not limited to, U.S. Pat. Nos. 6,308,170 and 6,228,575, the disclosures of which are herein incorporated by reference. Methods of comparing expression profiles are also described above.

A statistical analysis step is then performed to obtain the weighted contribution of the set of predictive genes. For example, nearest shrunken centroids analysis may be applied as described in Tibshirani et al. (2002) P.N.A.S. 99:6567-6572 to compute the centroid for each class, then compute the average squared distance between a given expression profile and each centroid, normalized by the within-class standard deviation.

The classification is probabilistically defined, where the cut-off may be empirically derived. In one embodiment of the invention, a probability of about 0.4 may be used to distinguish between quiescent and induced patients, more usually a probability of about 0.5, and may utilize a probability of about 0.6 or higher. A “high” probability may be at least about 0.75, at least about 0.7, at least about 0.6, or at least about 0.5. A “low” probability may be not more than about 0.25, not more than 0.3, or not more than 0.4. In many embodiments, the above-obtained information about the cell/tissue being assayed is employed to predict whether a host, subject or patient should be treated with a therapy of interest and to optimize the dose therein.

Various methods for analysis of a set of data may be utilized. In one embodiment, expression data is subjected to transformation and normalization. For example, ratios are generated by mean centering the expression data for each gene (by dividing the intensity measurement for each gene on a given array by the average intensity of the gene across all arrays), (2) then log-transformed (base 2) the resulting ratios, and (3) then median centered the expression data across arrays then across genes.

For cDNA microarray data, genes with fluorescent hybridization signals at least 1.5-fold greater than the local background fluorescent signal in the reference channel are considered adequately measured. The genes are centered by mean value within each dataset, and average linkage clustering carried out. The samples are segregated into two classes based on the first bifurcation in the hierarchical clustering “dendrogram”. The clustering and reciprocal expression of genes in tumor expression data allows classes of tumors to be unambiguously assigned.

To address the level of redundancy of STS genes in achieving tumor classification, a shrunken centroid analysis may be applied, using Prediction Analysis of Microarrays (PAM). Using a 10-fold balanced leave-one-out training and testing procedure, the minimum number of genes in an STS dataset that are sufficient to recapitulate the classification may be obtained.

A scaled approach may also be taken to the data analysis. Pearson correlation of the expression values of STS genes of tumor samples to the serum-activated fibroblast centroid results in a quantitative score reflecting the wound response signature for each sample. The higher the correlation value, the more the sample resembles serum-activated fibroblasts (“activated” wound response signature). A negative correlation value indicates the opposite behavior and higher expression of the “quiescent” wound response signature. The threshold for the two classes can be moved up or down from zero depending on the clinical goal.

The data may be subjected to non-supervised hierarchical clustering to reveal relationships among profiles. For example, hierarchical clustering may be performed, where the Pearson correlation is employed as the clustering metric. Clustering of the correlation matrix, e.g. using multidimensional scaling, enhances the visualization of functional homology similarities and dissimilarities. Multidimensional scaling (MDS) can be applied in one, two or three dimensions.

The analysis may be implemented in hardware or software, or a combination of both. In one embodiment of the invention, a machine-readable storage medium is provided, the medium comprising a data storage material encoded with machine readable data which, when using a machine programmed with instructions for using said data, is capable of displaying a any of the datasets and data comparisons of this invention. Such data may be used for a variety of purposes, such as drug discovery, analysis of interactions between cellular components, and the like. Preferably, the invention is implemented in computer programs executing on programmable computers, comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer may be, for example, a personal computer, microcomputer, or workstation of conventional design.

Each program is preferably implemented in a high level procedural or object oriented programming language to communicate with a computer system. However, the programs can be implemented in assembly or machine language, if desired. In any case, the language may be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system may also be considered to be implemented as a computer-readable storage medium, configured with a computer program, where the storage medium so configured causes a computer to operate in a specific and predefined manner to perform the functions described herein.

A variety of structural formats for the input and output means can be used to input and output the information in the computer-based systems of the present invention. One format for an output means test datasets possessing varying degrees of similarity to a trusted profile. Such presentation provides a skilled artisan with a ranking of similarities and identifies the degree of similarity contained-in the test pattern.

In situ hybridization and Immunohistochemistry. In addition to analysis based on bulk gene expression profiles, analysis may be performed based on in situ hybridization analysis, or antibody binding to tissue sections. Such analysis allows identification of histologically distinct cells within a tumor mass, and the identification of genes expressed in such cells. Such methods find particular interest value with the gene sets identified herein, in which expression is associated with the stromal cell component of a solid tumor. Criteria for selection of probes for in situ hybridization are discussed above.

Sections for hybridization may comprise one or multiple solid tumor samples, e.g. using a tissue microarray (see, for example, West and van de Rijn (2006) Histopathology 48(1):22-31; and Montgomery et al. (2005) Appl Immunohistochem Mol Morphol. 13(1):80-4). Tissue microarrays (TMAs) comprise multiple sections.

A selected probe, e.g. antibody specific for an STS gene product; or probe specific for an STS gene, is detectable labeled, and allowed to bind to the tissue section, using methods known in the art. The staining may be combined with other histochemical or immunohistochemical methods. The expression of selected genes in a stromal component of a tumor allows for characterization of the cells according to similarity to a stromal cell correlate of a soft tissue tumor.

Target Identification and Screening. Genes within the filtered STS gene set also provide a platform for target discovery. In some embodiments, a subset of genes is selected based on properties of the encoded protein, e.g. transmembrane domains, kinase domains, etc. Selection may also be based on the expression of a gene in the stromal component of one or more carcinomas, where desirable genes are expressed at high levels in such stromal components. In certain embodiments, it will be desirable to select genes that are not expressed or expressed at low levels in the corresponding transformed epithelial cells, e.g. in order to provide a complementary or synergistic drug target.

Target sequences can provide a platform for drug discovery. Compound screening may be performed using an in vitro model, a genetically altered cell or animal, or purified protein corresponding to an STS gene. One can identify ligands or substrates that bind to, modulate or mimic the action of the encoded polypeptide. Compound screening may be initially performed to determine candidate agents that bind to or otherwise interact with the target sequence, followed by a secondary screening that tests the activity of the compound In the context of a carcinoma or other solid tumor where a stromal component is present, e.g. in an animal model, xenotransplantation of tumors, in vitro tissue model, etc.

STS polypeptides include those encoded by genes in the provided gene sets, as well as nucleic acids that, by virtue of the degeneracy of the genetic code, are not identical in sequence to the disclosed nucleic acids, and variants thereof. Variant polypeptides can include amino acid (aa) substitutions, additions or deletions. The amino acid substitutions can be conservative amino acid substitutions or substitutions to eliminate non-essential amino acids, such as to alter a glycosylation site, a phosphorylation site or an acetylation site, or to minimize misfolding by substitution or deletion of one or more cysteine residues that are not necessary for function. Variants can be designed so as to retain or have enhanced biological activity of a particular region of the protein (e.g., a functional domain and/or, where the polypeptide is a member of a protein family, a region associated with a consensus sequence). Variants also include fragments of the polypeptides disclosed herein, particularly biologically active fragments and/or fragments corresponding to functional domains. Fragments of interest will typically be at least about 10 aa to at least about 15 aa in length, usually at least about 50 aa in length, and can be as long as 300 aa in length or longer, but will usually not exceed about 500 aa in length, where the fragment will have a contiguous stretch of amino acids that is identical to a polypeptide encoded by STS associated genes, or a homolog thereof.

Compound screening identifies agents that modulate function of the STS polypeptides. Of particular interest are screening assays for agents that have a low toxicity for human cells. A wide variety of assays may be used for this purpose. Knowledge of the 3-dimensional structure of the encoded protein, derived from crystallization of purified recombinant protein, could lead to the rational design of small drugs that specifically inhibit activity. These drugs may be directed at specific domains.

Functional assays are of interest, for example in investigating polypeptides associated with angiogenesis, the effect of an agent on an invasion assay may be monitored, for example, to provide a measure of the cells ability to move through a matrix like matrigel in response to a chemoattractant, e.g. 5% fetal bovine serum, etc. Percent Invasion is determined by the number of cells invading through matrigel coated FluoroBlok membrane divided by the number of cells invading through uncoated Fluorblok membrane. A number of in vitro and in vivo bioassays have been developed to mimic the complex process of angiogenesis. Among these, two assays in particular have been widely used to screen specifically for angiogenic regulatory factors, each mimicking an aspect of angiogenesis; namely, endothelial cell proliferation and migration. The proliferation assay uses cultured capillary endothelial cells and measures either increased cell number or the incorporation of radiolabeled or modified nucleosides to detect cells in S phase. In contrast, the chemotaxis assay separates endothelial cells and a test solution by a porous membrane disc (a Boyden Chamber), such that migration of endothelial cells across the barrier is indicative of a chemoattractant present in the test solution.

Rate of internalization can be measured by coupling a fluorescent tag to the protein for example using the Cellomics Array Scan HCS reader. Rate of association and dissociation can also be measured in a similar fashion. Receptor internalization can be measured by its accumulation in the recycling compartment, and the receptor's decrease in the recycling compartment.

Gelatin zymography is a qualitative method to analyze enzymes involved in matrix degradation. It can be combined with fluorogenic substrate assays to demonstrate temporal changes in enzyme concentration and activity. The invasive property of a tumor may be accompanied by the elaboration of proteolytic enzymes, such as collagenases, that degrade matrix material and basement membrane material, to enable the tumor to expand beyond the confines of the particular tissue in which that tumor is located. Elaboration of such enzymes may be by endogenous synthesis within the tumor cells, or may be elicited from adjacent cells or by circulating neutrophils, in which cases the elicitation by the tumor results from chemical messengers elaborated by the tumor and expression of the enzymes occurs at the tumor site or proximal to the tumor.

The effect of an agent on signaling pathways may be determined using reporter assays that well known in the art. Binding by a ligand triggers activation of key cell signaling pathways, such as p21.sup.ras, MAP kinases, NF-kappaB and cdc42/rac implicated in tumors. The cis reporting system can be used to determine if the gene or protein of interest acts on specific enhancer elements while the trans-activator indicates if the gene or protein of interest directly or indirectly may be involved in the phosphorylation and activation of the transcription factor.

The term “agent” as used herein describes any molecule, e.g. protein or pharmaceutical, with the capability of altering or mimicking the physiological function of an STS polypeptide. Generally a plurality of assay mixtures are run in parallel with different agent concentrations to obtain a differential response to the various concentrations. Typically one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Test agents can be obtained from libraries, such as natural product libraries or combinatorial libraries, for example. A number of different types of combinatorial libraries and methods for preparing such libraries have been described, including for example, PCT publications WO 93/06121, WO 95/12608, WO 95/35503, WO 94/08051 and WO 95/30642, each of which is incorporated herein by reference.

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin, etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The mixture of components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hours will be sufficient.

Preliminary screens can be conducted by screening for compounds capable of binding to a STS polypeptide, as at least some of the-compounds so identified are likely inhibitors. The binding assays usually involve contacting a STS polypeptide with one or more test compounds and allowing sufficient time for the protein and test compounds to form a binding complex. Any binding complexes formed can be detected using any of a number of established analytical techniques. Protein binding assays include, but are not limited to, methods that measure co-precipitation, co-migration on non-denaturing SDS-polyacrylamide gels, and co-migration on Western blots.

In response to a candidate agent, the level of expression or activity can be compared to a baseline value. As indicated above, the baseline value can be a value for a control sample or a statistical value that is representative of expression levels for a control population. Expression levels can also be determined for cells that do not express an STS gene, as a negative control. Such cells generally are otherwise substantially genetically the same as the test cells. Various controls can be conducted to ensure that an observed activity is authentic including running parallel reactions with cells that lack the reporter construct or by not contacting a cell harboring the reporter construct with test compound.

Compounds that are initially identified by any of the foregoing screening methods can be further tested to validate the apparent activity. The basic format of such methods involves administering a lead compound identified during an initial screen to an animal that serves as a model for humans and then determining if the desired activity is found. The animal models utilized in validation studies generally are mammals. Specific examples of suitable animals include, but are not limited to, primates, mice, and rats.

Tumor classification and patient stratification. The invention provides for methods of classifying tumors, and thus grouping or “stratifying” patients, according to the STS signature. As shown in the Examples, tumors classified as having a particular signature carry a higher risk of metastasis and death, and therefore may be treated more aggressively than tumors of a less aggressive type.

The tumor of each patient in a pool of potential patients for a clinical trial can be classified as described above. Patients having similarly classified tumors can then be selected for participation in an investigative or clinical trial of a cancer therapeutic where a homogeneous population is desired. The tumor classification of a patient can also be used in assessing the efficacy of a cancer therapeutic in a heterogeneous patient population. Thus, comparison of an individual's expression profile to the population profile for a type of cancer, permits the selection or design of drugs or other therapeutic regimens that are expected to be safe and efficacious for a particular patient or patient population (i.e., a group of patients having the same type of cancer).

The methods of the invention can be carried out using any suitable probe for detection of a gene product that is differentially expressed in cancer cells. For example, mRNA (or cDNA generated from mRNA) expressed from a STS gene can be detected using polynucleotide probes. In another example, the STS gene product is a polypeptide, which polypeptides can be detected using, for example, antibodies that specifically bind such polypeptides or an antigenic portion thereof.

The present invention relates to methods and compositions useful in diagnosis of cancer, design of rational therapy, and the selection of patient populations for the purposes of clinical trials. The invention is based on the discovery that tumors of a patient can be classified according to STS expression profile. Polynucleotides that correspond to the selected STS genes can be used in diagnostic assays to provide for diagnosis of cancer at the molecular level, and to provide for the basis for rational therapy (e.g., therapy is selected according to the expression pattern of a selected set of genes in the tumor). The gene products encoded by STS genes can also serve as therapeutic targets, and candidate agents effective against such targets screened by, for example, analyzing the ability of candidate agents to modulate activity of differentially expressed gene products.

### Databases of Expression Profiles

Also provided are databases of expression profiles of STS genes. Such databases will typically comprise expression profiles derived from soft tissue tumors of interest, carcinoma cell samples, normal soft tissue samples, etc. The expression profiles and databases thereof may be provided in a variety of media to facilitate their use. “Media” refers to a manufacture that contains the expression profile information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure may be chosen, based on the means used to access the stored information. A variety of data processor programs and formats can be used for storage, e.g. word processing text file, database format, etc.

As used herein, “a computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

A variety of structural formats for the input and output means can be used to input and output the information in the computer-based systems of the present invention. Such presentation provides a skilled artisan with a ranking of similarities and identifies the degree of similarity contained in the test expression profile.

### Reagents and Kits

Also provided are reagents and kits thereof for practicing one or more of the above-described methods. The subject reagents and kits thereof may vary greatly. Reagents of interest include reagents specifically designed for use in production of the above described expression profiles of phenotype determinative genes.

One type of such reagent is an array of probe nucleic acids in which STS genes of interest are represented. A variety of different array formats are known in the art, with a wide variety of different probe structures, substrate compositions and attachment technologies. Representative array structures of interest include those described in U.S. Pat. Nos.: 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In certain embodiments, the number of genes that are from that is represented on the array is at least 10, usually at least 25, and may be at least 50, 100, up to including all of the STS genes, preferably utilizing the top ranked set of genes. Where the subject arrays include probes for such additional genes, in certain embodiments the number % of additional genes that are represented does not exceed about 50%, usually does not exceed about 25%.

Another type of reagent that is specifically tailored for generating expression profiles of STS genes is a collection of gene specific primers that is designed to selectively amplify such genes, for use in quantitative PCR and other quantitation methods. Gene specific primers and methods for using the same are described in U.S. Pat. No. 5,994,076, the disclosure of which is herein incorporated by reference. Of particular interest are collections of gene specific primers that have primers for at least 10 of the STS genes, often a plurality of these genes, e.g., at least 25, and may be 50, 100 or more to include all of the STS genes. The subject gene specific primer collections may include only STS genes, or they may include primers for additional genes.

The kits of the subject invention may include the above described arrays and/or gene specific primer collections. The kits may further include a software package for statistical analysis of one or more phenotypes, and may include a reference database for calculating the probability of susceptibility. The kit may include reagents employed in the various methods, such as primers for generating target nucleic acids, dNTPs and/or rNTPs, which may be either premixed or separate, one or more uniquely labeled dNTPs and/or rNTPs, such as biotinylated or Cy3 or Cy5 tagged dNTPs, gold or silver particles with different scattering spectra, or other post synthesis labeling reagent, such as chemically active derivatives of fluorescent dyes, enzymes, such as reverse transcriptases, DNA polymerases, RNA polymerases, and the like, various buffer mediums, e.g. hybridization and washing buffers, prefabricated probe arrays, labeled probe purification reagents and components, like spin columns, etc., signal generation and detection reagents, e.g. streptavidin-alkaline phosphatase conjugate, chemifluorescent or chemiluminescent substrate, and the like.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

The above-described analytical methods may be embodied as a program of instructions executable by computer to perform the different aspects of the invention. Any of the techniques described above may be performed by means of software components loaded into a computer or other information appliance or digital device. When so enabled, the computer, appliance or device may then perform the above-described techniques to assist the analysis of sets of values associated with a plurality of genes in the manner described above, or for comparing such associated values. The software component may be loaded from a fixed media or accessed through a communication medium such as the internet or other type of computer network. The above features are embodied in one or more computer programs may be performed by one or more computers running such programs.

### Diagnosis, Prognosis, Assessment of Therapy (Therametrics), and Management of Cancer

The classification methods described herein, as well as their gene products and corresponding genes and gene products, are of particular interest as genetic or biochemical markers (e.g., in blood or tissues) that will detect the earliest changes along the carcinogenesis pathway and/or to monitor the efficacy of various therapies and preventive interventions.

Staging. Staging is a process used by physicians to describe how advanced the cancerous state is in a patient. Staging assists the physician in determining a prognosis, planning treatment and evaluating the results of such treatment. Staging systems vary with the types of cancer, but generally involve the following “TNM” system: the type of tumor, indicated by T; whether the cancer has metastasized to nearby lymph nodes, indicated by N; and whether the cancer has metastasized to more distant parts of the body, indicated by M. Generally, if a cancer is only detectable in the area of the primary lesion without having spread to any lymph nodes it is called Stage I. If it has spread only to the closest lymph nodes, it is called Stage II. In Stage III, the cancer has generally spread to the lymph nodes in near proximity to the site of the primary lesion. Cancers that have spread to a distant part of the body, such as the liver, bone, brain or other site, are Stage IV, the most advanced stage.

The methods described herein can facilitate fine-tuning of the staging process by identifying the aggressiveness of a cancer, e.g. the metastatic potential, as well as the presence in different areas of the body. Thus, a Stage II cancer with a classification signifying a high metastatic potential cancer can be used to change a borderline Stage II tumor to a Stage III tumor, justifying more aggressive therapy. Conversely, the presence of a polynucleotide signifying a lower metastatic potential allows more conservative staging of a tumor.

The following examples are offered by way of illustration and not by way of limitation.

### EXAMPLE 1

### Determination of Stromal Signatures in Breast Carcinoma

Many soft tissue tumors recapitulate features of normal connective tissue. Different types of fibroblastic tumors are shown herein to be representative of different populations of fibroblastic cells or different activation states of these cells. We examined two tumors with fibroblastic features, solitary fibrous tumor (SFT) and desmoid-type fibromatosis (DTF), by DNA microarray analysis and found that they have very different expression profiles, including significant differences in their patterns of expression of extracellular matrix genes and growth factors. Using immunohistochemistry and in situ hybridization on a tissue microarray, we found that genes specific for these two tumors have mutually specific expression in the stroma of nonneoplastic tissues. We defined a set of 786 gene spots whose pattern of expression distinguishes SFT from DTF. In an analysis of DNA microarray gene expression data from 295 previously published breast carcinomas, we found that expression of this gene set defined two groups of breast carcinomas with significant differences in overall survival. One of the groups had a favorable outcome and was defined by the expression of DTF genes. The other group of tumors had a poor prognosis and showed variable expression of genes enriched for SFT type. Our findings suggest that the host stromal response varies significantly among carcinomas and that gene expression patterns characteristic of soft tissue tumors can be used to discover new markers for normal connective tissue cells.

Numerous soft tissue tumors demonstrate specific differentiation toward connective tissue. This may be represented in cytoplasmic organelles or extracellular matrix deposition, or defined by immunohistochemical features. Some soft tissue tumors have features of smooth muscle cells (leiomyomas, leiomyosarcomas) or adipocytes (lipoma, liposarcoma). Other soft tissue tumors exhibit features of rarer cell types such as the interstitial cell of Cajal (gastrointestinal stromal tumor) and glomus cells (glomus tumor). There are numerous tumors with fibroblastic and myofibroblastic features, but their corresponding normal counterparts are not well delineated by available markers. We examined two fibroblastic tumors: solitary fibrous tumor (SFT) and desmoid-type fibromatosis (DTF). Both tumors are composed of spindled cells, typically have low-grade nuclear morphology, and can occur throughout the body. Most SFTs occur on the pleural surface, but they have been recognized in a wide range of anatomic locations. Although they were initially thought to be associated with mesothelial differentiation, a number of studies have indicated that SFTs are derived from fibroblasts. The vast majority of SFTs are CD34 immunoreactive. SFTs do not generally infiltrate into surrounding soft tissue, recur after excision, or metastasize. However, a minority of cases exhibit malignant features and these are associated with chromosomal alterations.

DTF is widely assumed to be derived from fibroblasts of the deep soft tissue. DTFs occur both sporadically or as part of a syndrome due to germline APC mutations in familial adenomatous polyposis coli. These tumors are often found in the deep soft tissue of the trunk or abdomen. The sporadic DTFs also often have mutations in APC or b-catenin, suggesting that abnormal activation of the canonical Wnt pathway plays a role in their pathogenesis. Sporadic and familial DTFs have been found to be composed of a monoclonal population. DTFs are locally aggressive and are difficult to resect completely: local recurrences in anatomically critical sites can be fatal.

Thus SFT and DTF show significant differences in clinical behavior. Although the histologic growth patterns are distinct, with DTF showing a more aggressive infiltrative growth than SFT, the individual cells that comprise these tumors are histologically very similar and hard to distinguish. As such, these two tumors form a good model system to use for discovery of novel connective tissue markers.

In this study, we used DNA microarrays to profile gene expression of two fibroblastic tumors, DTF and SFT. The gene expression profiles define two different fibroblastic neoplasms that corresponds to two physiologic fibroblastic phenotypes or fibroblastic response patterns. We demonstrate that several genes differentially expressed in DTF and SFT are also differentially expressed in characteristic patterns in conditions from inflammatory and reparative tissue to neoplasia. Here we show that gene sets discovered in fibroblastic tumors can be used to recognize prognostically distinct subsets of breast carcinomas.

Results

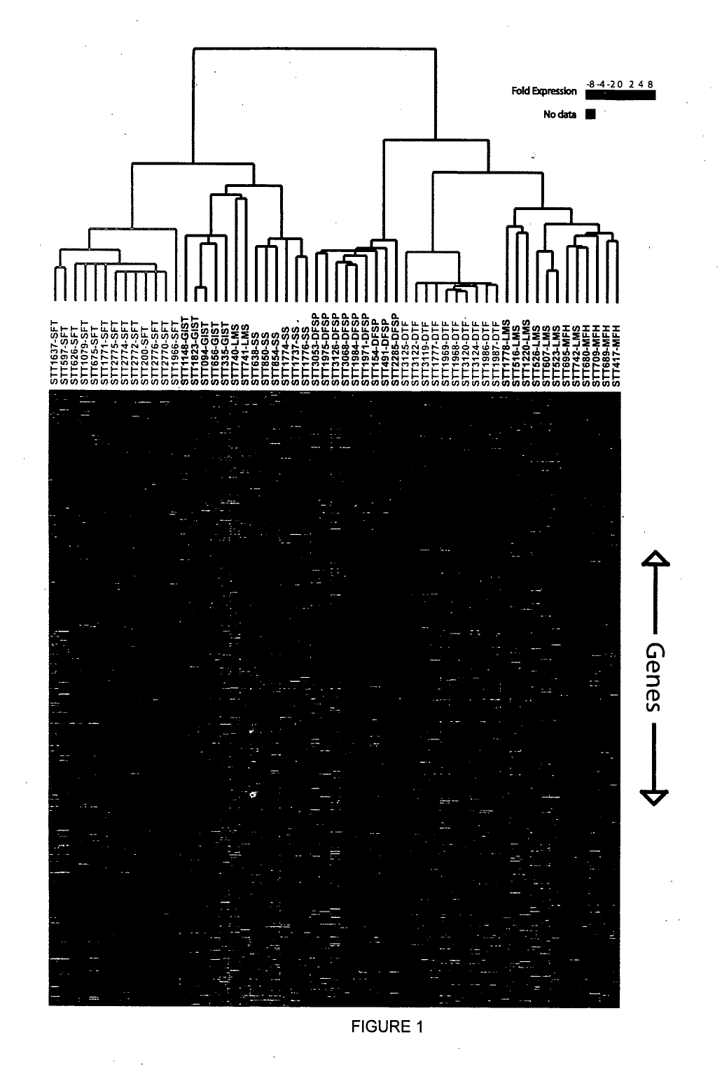

Expression Profiling Comparison of SFT and DTF. The ten cases of DTF and 13 cases of benign SFT were compared to 35 other previously examined soft tissue tumors with expression profiling on 42,000-element cDNA microarrays, corresponding to approximately 36,000 unique gene sequences. Unsupervised hierarchical cluster analysis organized the 58 tumors and the 3,778 gene spots that demonstrate at least 4-fold variation from the mean in at least two tumors. Based on gene expression, all the DTF and SFT cases can be separated into two groups according to the pathologic diagnosis. The two fibroblastic tumors did not group together. Instead, the SFTs clustered on the same branch as synovial sarcoma and gastrointestinal stromal tumor, whereas the DTF cases clustered on the same branch as the majority of leiomyosarcomas, dermatofibrosarcoma protuberans, and malignant fibrous histiocytomas (FIG. 1).

Comparison of Expression Patterns in SFT and DTF. To directly compare the expression patterns, the ten cases of DTF and 13 cases of SFT were analyzed without the other soft tissue tumors. Using the same filtering criteria as above, the 23 tumors were clustered based on 1,010 gene spots. Again, the tumors clustered according to pathologic diagnosis. The dataset was analyzed using the significance analysis of microarray (SAM) method to create two lists. The two lists included genes significantly more highly expressed in either SFT or DTF. A total of 786 gene spots, differentially expressed between the two tumor types, had a false discovery rate of one in 786 (0.13%). The SFT-specific gene list shared 64% identity with a list of genes selected using SAM for specific expression in SFT compared to all other soft tissue tumors in the initial set of 58 soft tissue tumors. Likewise, the DTF-specific gene list shared 65% identity with a list selected by SAM based on differential expression in DTF compared with the 58 soft tissue tumors.

The two tumor types differed in their patterns of expression in a number of different functional categories of genes, for example as shown in Table 1.

| | Extracellular Matrix | | | Growth Factor Pathways | | | WNT Pathway | | |
|---|---|---|---|---|---|---|---|---|---|
| Category | Symbol | UGCluster | LLID | Symbol | UGCluster | LLID | Symbol | UGCluster | LLID |
| Selected fibromatosis genes | COL1A1 | Hs.172928 | 1277 | TGFB2 | Hs.133379 | 7042 | FZD1 | Hs.94234 | 8321 |
| | COLSA1 | Hs.210283 | 1289 | TGFB3 | Hs.2025 | 7043 | FZD2 | Hs.142912 | 2535 |
| | COL3A1 | Hs.443625 | 1281 | CTGF | Hs.410037 | 1490 | DKK2 | Hs.211869 | 27123 |
| | COL6A1 | Hs.474053 | 1291 | FGF11 | Hs.528468 | 2256 | DKK3 | Hs.292156 | 27122; 10530 |
| | COL8A1 | Hs.134830 | 1295 | FGF12 | Hs.185577 | 2257 | WISP1 | Hs.492974 | 8840 |
| | FBN1 | Hs.146447 | 2200 | | | | WISP2 | Hs.194679 | 8839 |
| | TPM1 | Hs.133892 | 7168 | | | | WNT5A | Hs.152213 | 7474 |
| | MYL9 | Hs.504687 | 10398 | | | | | | |
| | MYO10 | Hs.481720 | 4651 | | | | | | |
| | CNN1 | Hs.465929 | 1264 | | | | | | |
| | CALD1 | Hs.490203 | 800 | | | | | | |
| | ADAM12 | Hs.386283 | 8038 | | | | | | |
| | ADAM19 | Hs.483944 | 8728; 26999 | | | | | | |
| | ADAMTS1 | Hs.534115 | 9510 | | | | | | |
| | MMP11 | Hs.143751 | 4320 | | | | | | |
| | MMP19 | Hs.154057 | 4327 | | | | | | |
| | MMP23b | Hs.211819 | 8510; 8511 | | | | | | |
| Selected SFT genes | COL4A5 | Hs.369089 | 1287 | NRG2 | Hs.408515 | 9542 | | | |
| | COL17A1 | Hs.117938 | 1308 | ERBB2 | Hs.446352 | 2064 | | | |
| | COL21A1 | Hs.47629 | 81578 | EPS8 | Hs.26139 | 2059 | | | |
| | CDH24 | Hs.155912 | 64403 | DDR1 | Hs.520004 | 780 | | | |
| | SPOCK | Hs.124611 | 6695 | MERTK | Hs.306178 | 10461 | | | |
| | SPOCK3 | Hs.481133 | 50859 | IGF1 | Hs.160562 | 3479 | | | |

DTF and SFT were analyzed by SAM (see Materials and Methods) resulting in 786 genes with fewer than 0.1% false positive genes. Entire gene list is available at http://microarray.pubs.stanford.edu/ [symbol] portal/DTF_SFTbreast
DOt: 10.1.371/journal.pbio.0030187.t001

On the basis of these differences in expression, the cells of origin for each lesion may perform different functions in normal connective tissue. One of the more striking differences is in the variation of genes involved in fibrotic response and basement membrane synthesis between the two tumors. DTF has high expression of genes involved in the fibrotic response. These include numerous collagen genes, such as COL1A1 and COL3A1, involved in fibrosis and contraction and a number of growth factors that stimulate the classic fibrotic response. DTFs also highly express numerous genes that remodel the extracellular matrix, including ADAM and MMP family members, consistent with its infiltrative behavior. In contrast, SFTs highly express collagen genes and other genes involved in basement membrane formation and maintenance, such as COL4A5 and COL17A1. In contrast to DTF, no metalloproteinase family members were especially highly expressed in SFTs. Possible exceptions were ADAM22 and ADAM23, which were highly expressed in SFT. But the metalloprotease domain is inactive in these proteins, and these proteins are more likely involved in cell adhesion than in matrix remodeling. SFTs highly express a number of signaling pathways involved in growth and survival, including BCL2 and IGF1. DTF and SFT also differed in other pathways, including WNT signaling and THY1 expression. Thus, although SFT and DTF both express genes typically expressed in fibroblasts, they express genes that belong to very different functional groups.

Histologic Patterns of Expression of Genes Characteristic of SFT and DTF. To confirm, localize, and extend our observations on the expression of DTF- and SFT-specific genes, we constructed a tissue microarray (TMA) and measured expression using immunohistochemistry (IHC) and in situ hybridization. The TMA contained representative cores of five DTFs and SFTs, in addition to cores of scar and keloid. In addition, the TMA included well-oriented embedded pieces of normal skin, lung, and breast tissue. The array also contained 11 fibroadenomas, as well as five colorectal and 24 breast carcinomas.

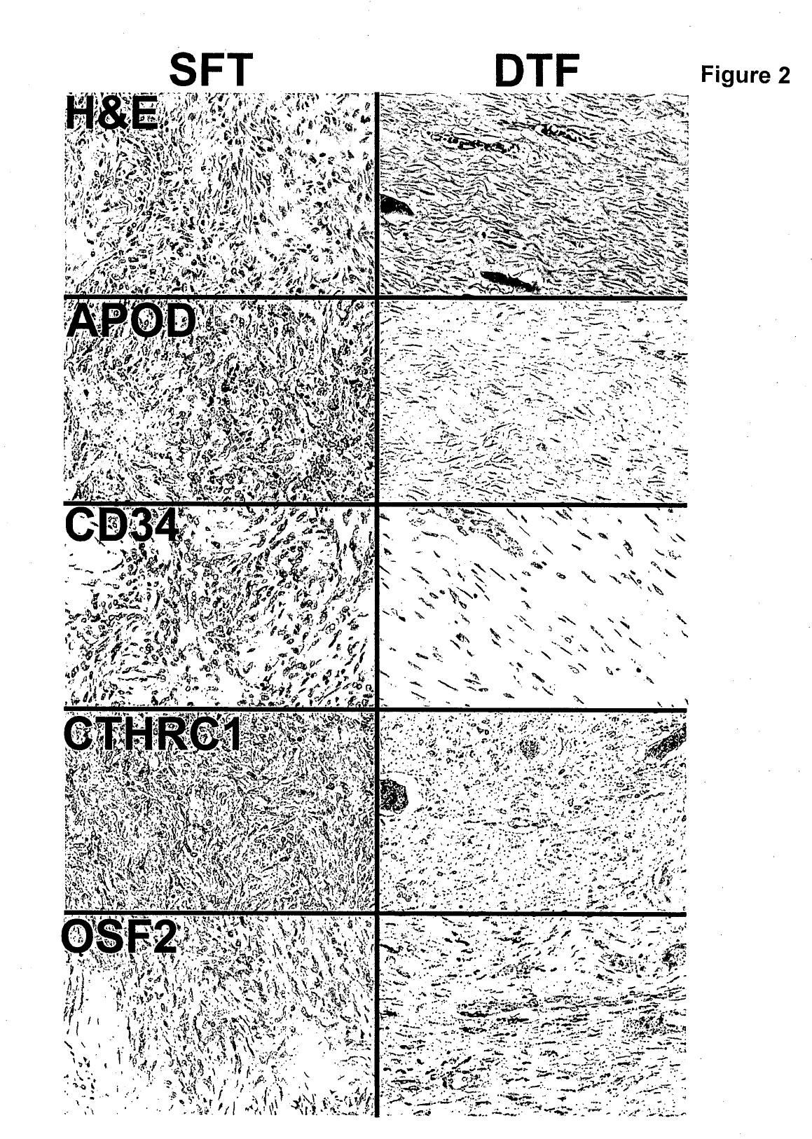

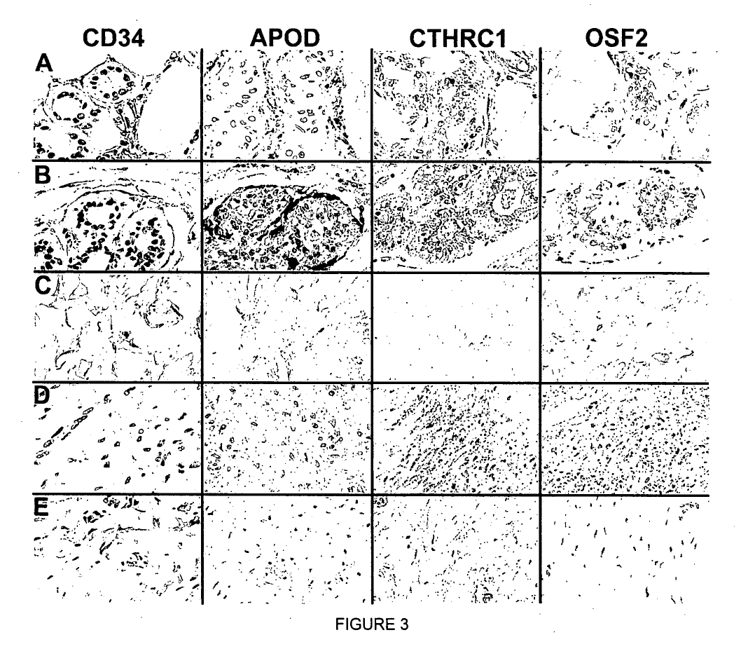

SFTs, fibroadenomas, and a subset of normal fibroblasts in the skin and breast specimens demonstrated expression of SFT-specific genes (FIGS. 2 and 3). Normal fibroblasts that reacted for SFT markers, APOD and CD34, included those associated with adnexal glands and dermal fat. The reactivity of so-called dendritic interstitial cells for CD34 in a, number of locations was previously reported. These tissues were rarely positive for DTF-specific gene probes. DTF-specific probes, for OSF2 and CTHRC1, were positive in DTF, keloid, scar, granulation tissue, and fistula tract (FIGS. 2 and 3). In the granulation tissue and fistula tract tissue, a gradient of expression dependent on location of the cells within the tissue could be identified in some hybridizations. There was no staining of fibroblast-like cells by probes for OSF2 and CTHRC1 in the normal tissues.

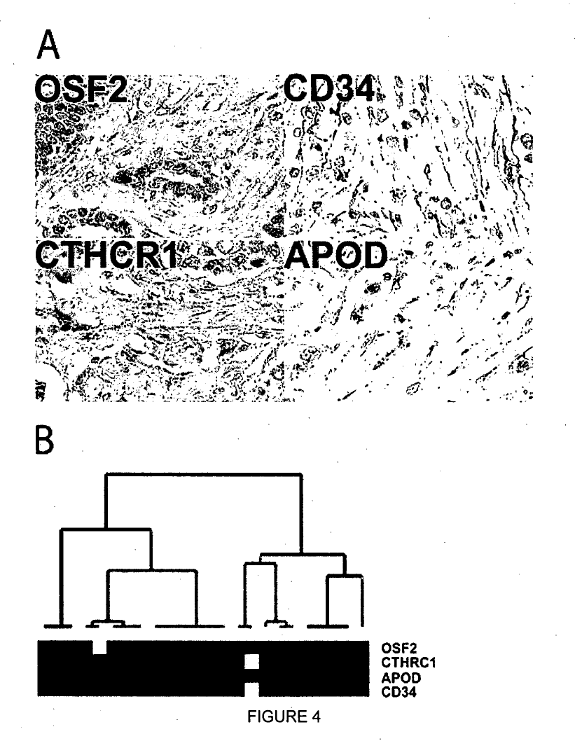

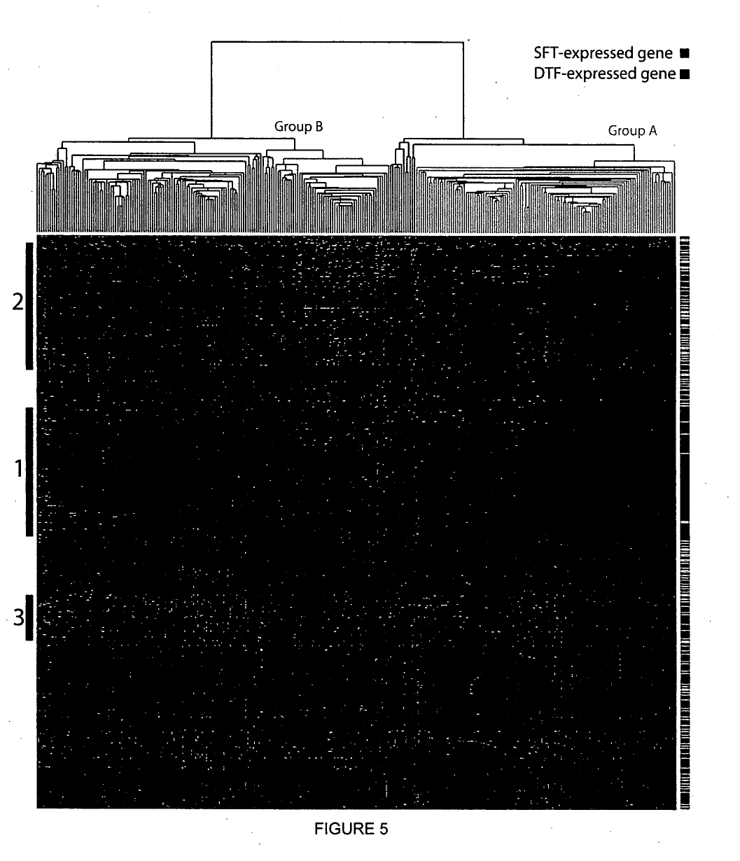

A similar pattern of differential expression of SFT and DTF markers was observed in breast carcinoma. With the exception of APOD, only stromal staining was observed with these markers whereas the neoplastic epithelial cells did not react. For breast carcinoma, 24 cases were scored for stromal staining and clustered by hierarchical clustering. The resulting dendrogram and heatmap are shown in FIG. 4. A subset of cases was positive for the SFT markers, CD34 and APOD, another for the DTF markers, OSF2 and CTHRC1.

Variable Expression of Genes Characteristic of Fibroblastic Tumors in Breast Carcinoma. To further investigate the implication of the variation in expression of these fibroblastic tumor-related genes in breast cancer, we analyzed their expression in 295 breast carcinomas using a previously published dataset. We focused on the genes selected by SAM for differential expression in DTF versus SFT, and investigated their expression levels in the published breast cancer dataset.

When clustering the breast carcinomas with the fibroblastic tumor-related genes only, the resulting dendrogram of the tumors/samples showed several high-order branches of correlation between distinct tumor groups. Two of these groups (FIG. 5, groups A and B) showed remarkable differences in the expression of DTF versus SFT genes. Tumor group A, composed of 120 breast carcinomas, showed high levels of expression of a gene cluster (gene cluster 1, left sidebar) highly enriched for genes that are found in DTF (see right sidebar: genes highly expressed in DTF are represented by purple). This gene cluster was predominately composed of genes whose protein products interact with the extracellular matrix, including collagens, cadherins, and remodeling enzymes. Moreover, two key growth factors in the fibrotic response were also identified, TGFB3 and CTGF. The second tumor group (group B), composed of 59 breast carcinomas, showed expression of a mixture of genes (gene cluster 2, left sidebar) that were enriched for those genes that positively identified SFT (see right sidebar: genes highly expressed in SFT are represented by pink). This gene cluster contained extracellular matrix-interacting genes, such as COL9A3 and ADAMTS1. An additional cluster (gene cluster 3, left sidebar), containing a mixture of SFT and DTF genes, was predominately highly expressed across all tumors except for the tumor group B.

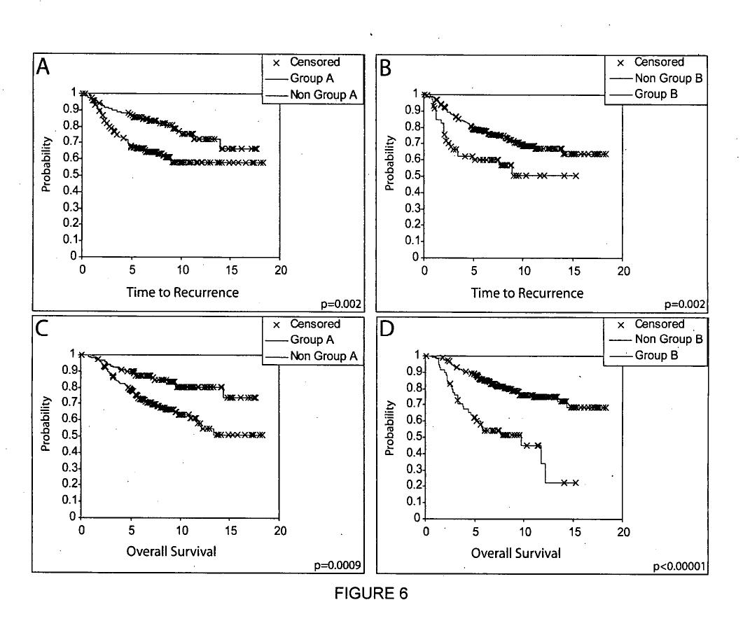

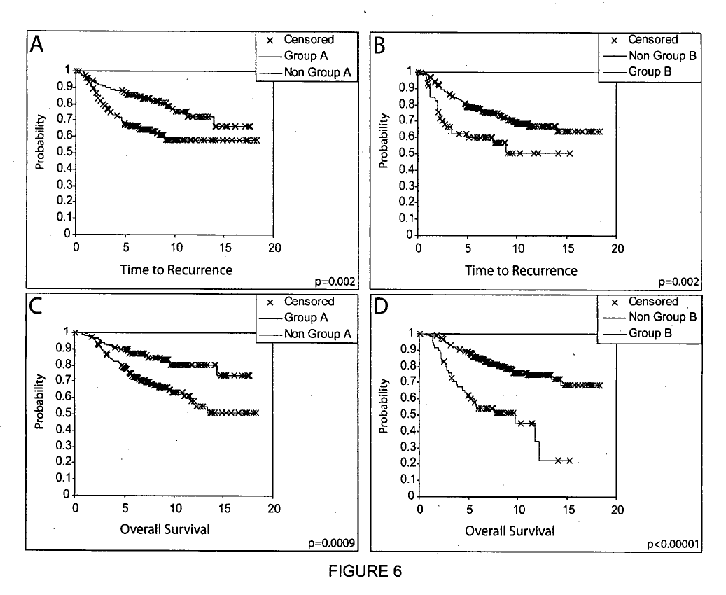

The prognosis of these two tumor groups, (A and B), was assessed by distant metastasis-free survival and overall survival (FIG. 6). Group A demonstrated significantly better outcomes in both overall survival (80% at 10 y vs. 63%; p=0.0009) and metastasis-free survival (77% at 10 y vs. 58%; p=0.002) as compared to the all tumors. In contrast, group B demonstrated significantly poorer outcome in overall survival (45% at 10 y vs. 76%; p<0.00001) and distant metastasis-free survival (50% at 10 y vs. 69%; p=0.002) compared to all other tumors.

For both tumor groups A and B, prognostic performance was independent in multivariate analysis for clinical risk factors including tumor size, lymph node status, and tumor grade (see Table 2). The hazard ratio for death was 2.6 (1.6-4.4, 95% confidence interval [CI]) for group B and 0.55 (0.33-0.92, 95% CI) for group A. Group B also retained independent prognostic relevance when the previously described 70-gene prognosis profile is considered in the model.

TABLE 2
Multivariate Analysis for Tumor Group Status versus Clinical Risk
Factors including Treatment with Chemotherapy, Tumor Size (<2 cm),
Lymph Node Status, Tumor Grade (Low and Intermediate versus
High), Age (<40 y old), Vascular Invasion

| Cox Regression Analysis Group and | | Statistical | | 95.0% Cl | |
|---|---|---|---|---|---|
| Clinical Risk Factors on Survival | Risk Factor | Significance | Hazard Ratio | Lower | Upper |
| Group A | ChemoTx | 0.355 | 1.386 | 0.694 | 2.768 |
| | Tumor size | 0.036 | 1.663 | 1.034 | 2.676 |
| | LN status | 0.622 | 1.181 | 0.61 | 2.284 |
| | Grade | 0.001 | 2.308 | 1.41 | 3.779 |
| | Age | 0.012 | 0.543 | 0.337 | 0.876 |
| | Vascular | 0.009 | 1.379 | 1.085 | 1.752 |
| | Group A | 0.021 | 0.549 | 0.33 | 0.915 |
| Group B | ChemoTx | 0.174 | 1.629 | 0.806 | 3.292 |
| | Tumor size | 0.076 | 1.551 | 0.954 | 2.52 |
| | LN status | 0.382 | 1.345 | 0.691 | 2.618 |
| | Grade | 0.013 | 1.934 | 1.15 | 3.255 |
| | Age | 0.013 | 0.547 | 0.339 | 0.882 |
| | Vascular | 0.001 | 1.502 | 1.178 | 1.916 |
| | Group B | 0.0002 | 2.62 | 1.577 | 4.353 |
| Group B and “70 genes” | ChemoTx | 0.18 | 1.624 | 0.799 | 3.298 |
| | Tumor size | 0.045 | 1.635 | 1.011 | 2.644 |
| | LN status | 0.55 | 1.225 | 0.629 | 2.385 |
| | Grade | 0.284 | 1.327 | 0.791 | 2.226 |
| | Age | 0.082 | 0.653 | 0.404 | 1.056 |
| | Vascular | 0.004 | 1.444 | 1.128 | 1.848 |
| | “70 genes” | <0.0001 | 5.249 | 2.284 | 12.059 |
| | Group B | 0.016 | 1.859 | 1.124 | 3.075 |

The hazard ratio for death, Cl and statistical significance are included.
The “70 genes” factor refers to the 70 genes previously published to be predictive in the 295 breast carcinomas dataset [15].
ChemoTx, chemotherapy,
LN lymph node.
DOt: 10.1371/journal.pbio.0030187.g002

Expression patterns among fibroblasts in tumors/carcinomas in vivo are difficult to assess due to tissue heterogeneity, which includes the relative content of epithelial cells, vascular structures, and inflammatory cells, and the diversity of fibroblastic and myofibroblastic cells that may be present. We have attempted to gain insight into the possible variation in expression patterns in fibroblastic cells by examining two fibroblastic neoplasms, SFT and DTF.

Soft tissue tumors are comprised of relatively pure populations of cells in comparison with other tissue types, including normal tissues and other neoplasms. Thus, the gene expression profile of a soft tissue tumor represents primarily a single cell type. To a degree, many soft tissue tumors recapitulate normal tissue components both morphologically and by protein expression, and this is the basis for much of the diagnostic nomenclature in surgical pathology.

We hypothesized that tumors with different fibroblastic features might represent different activation states or different subtypes of normal fibroblasts or stromal cells. Thus, we examined two tumors with fibroblastic differentiation: SFT and DTF. These two tumors have been extensively studied by morphology, IHC, and electron microscopy and are known to share features with non-neoplastic fibroblasts. In this study we demonstrate that the gene expression patterns of these two tumors are distinguished by differences in expression of a variety of functional groups of genes. DTF expresses numerous collagens that are present in a fibrotic response. Numerous myofibroblastic genes are also expressed by DTF. In contrast, SFTs express collagens and other extracellular matrix proteins that are typically found in the basement membrane. DTF tumors express several genes in the ADAM and MMP families involved in extracellular matrix remodeling, which might be relevant to the more infiltrative behavior of these tumors. SFTs expresses few of these genes, and the ADAMs that are expressed in SFT (ADAM22 and ADAM23) are probably involved more in cell adhesion than in extracellular matrix remodeling. In addition, DTF tumors express growth factors involved in the profibrotic response, such as TGFB and CTGF.

By IHC and ISH, markers representative of the separate DTF and SFT gene sets highlighted at least two groups of normal connective tissue “fibroblasts” or stromal cells. The cells positive for DTF markers are found in a variety of reactive tissues, ranging from inflammatory granulation tissue to scar tissue. In contrast, cells positive for SFT markers tend to be found in normal tissue. The stromal cells surrounding breast lobules and eccrine lobules of the skin were strongly reactive for SFT markers and negative for DTF genes. These findings are consistent with the gene expression data in which SFTs highly express many genes that help create basement membrane.

We created two gene sets consisting of genes that are positively identified either as DTF or SFT. For four genes we determined the expression patterns in breast carcinoma samples and showed that they were restricted to connective tissue cells and were not expressed by tumor cells. With these gene sets, we can evaluate for the presence of an expression signature of either SFT or DTF in other gene array datasets. In this study, we examined a previously published breast carcinoma dataset that contains 295 tumors with a median follow-up of 7.8 y. These gene sets highlight a minor expression pattern within a gene expression dataset that may not be readily apparent when the entire dataset is examined. In this case, the expression pattern is putatively associated with stromal fibroblast-like cells, a cell population that is often the minority in breast carcinoma and may not have as much RNA expression. Thus, we might expect the expression signature of stromal cells to be obscured in the hierarchical clustering of the entire dataset.

When the breast carcinoma dataset was analyzed with the SFT and DTF gene sets, three main gene clusters were apparent, one more tightly correlated than the other two. The first gene cluster (see FIG. 5, gene cluster 1) was composed almost entirely of DTF genes. Most of these genes are involved in stimulating or interacting with the extracellular matrix in a pro-fibrotic manner. This gene cluster identified a tumor cluster of 120 cases (tumor group A). Tumor group B showed a less-obvious relationship to either of the soft tissue tumors. However, it was defined by two gene clusters enriched for SFT genes, either by high expression for the genes (gene cluster 2) or relatively low expression for these genes (gene cluster 3). Interestingly, the two tumor groups had very different clinical behaviors. Tumor group A had a statistically significant better overall survival and metastasis-free survival when compared to the rest of the dataset. In contrast, tumor group B had a statistically significant worse overall survival and metastasis-free survival when compared to the rest of the dataset. In multivariate analysis this predictive value is independent of clinico-pathological risk factors. These findings show that stromal expression patterns can vary amongst breast carcinomas and may be clinically significant.

In summary, analysis of gene expression patterns in two soft tissue tumors, DTF and SFT, has allowed identification of at least two different nonneoplastic subtypes of stromal cells. Furthermore, analysis of the gene expression signatures of these soft tissue tumors in a breast carcinoma expression dataset has suggested that there may be molecularly distinct patterns of stromal reaction in breast cancer. These stromal reaction patterns appear to be correlated with differences in the biology of the tumors that are reflected in clinical outcome.

Materials and Methods

Tumor samples for DTF and SFT cDNA microarray analysis. Tumors were collected from four academic institutions with IRB approval. After resection, a representative sample was quickly frozen and stored at −80° C. Prior to processing, frozen sections of the tissue were cut and histologically examined to ensure that the tissue represented the diagnostic entity. The DTFs were all sporadic cases, including five cases from the extremities, two cases from the abdomen, two cases from the sacrum, and one case from the chest wall. The SFTs included 13 cases with benign features; all but one were derived from the chest cavity. SFT cases with malignant pathologic or clinical features were excluded. The diagnoses were based on clinical data, morphologic data, and IHC, including CD34.

DTF and SFT cDNA microarray procedures. We used 42,000-spot cDNA microarrays to measure the relative mRNA expression levels in the tumors. The details of isolating mRNA, labeling, and hybridizing are described in Linn et al. (2003) Am J Pathol 163: 2383-2395. Data were filtered using the following criteria: Only cDNA spots with a ratio of signal over background of at least 1.5 in both the Cy3 and the Cy5 channel were included; only cDNAs were selected that had an absolute value at least four times greater in at least two arrays than the geometric mean; and only cDNA spots that fulfill these criteria on at least 70% of the arrays were included. Data were evaluated with unsupervised hierarchical clustering and SAM.

Analysis of breast carcinoma dataset. The gene array dataset for breast carcinoma contained 295 tumors arrayed on 25,000-spot oligo nucleotide arrays as described by van de Vijver et al. (2002) N Engl J Med 347: 1999-2009. In short, patients were all diagnosed and treated in the Netherlands Cancer Institute for early breast cancer (Stage I and II) between 1984 and 1995. The median follow-up for living patients is 7.8 y.

For DTF and SFT, genes were identified that were highly expressed in either of the two tumor types by using SAM. A total of 1,010 spots satisfied the gene-filtering criteria mentioned above in-the clustering of the DTF and SFT tumors. The criterion for SAM was set to yield 0.1% false-positive data. A list of 786 clones was obtained that consisted of 493 genes positively identifying fibromatosis and 293 genes positively identifying SFT. Equal numbers of DTF and SFT clones were chosen for breast carcinoma analysis, and clones having the same Unigene locus were removed, resulting in 237 unique gene sequences identifying DTF and 246 unique gene sequences identifying SFT. These gene sequences were mapped to spots on the NKI array using Unigene build 172 (release date 17 Jul. 2004) to give 471 unique spots. Gene measurements were mean centered. The resulting dataset was subjected to hierarchical clustering with average linkage clustering.

Significant Genes List

| Input Parameters | Imputation Engine | 10-Nearest Neighbor Imputer |
|---|---|---|
| | Data Type | Two Class, unpaired data |
| | Data in log scale? | TRUE |
| | Number of Permutations | 100 |
| | Blocked Permutation? | FALSE |
| | RNG Seed | 1234567 |
| | (Delta, Fold Change) | (0.70329,) |
| | (Upper Cutoff, Lower Cutoff) | (1.02967, −2.08700) |
| Computed Quantities | Computed Exchangeability Factor S0 | 0.444648738 |
| | S0 percentile | 0.61 |
| | False Significant Number (Median, 90 percentile) | (1.02970, 8.77624) |
| | False Discovery Rate (Median, 90 percentile) | (0.13101, 1.11657) |
| | Pi0Hat | 0.07921 |

293 SFT genes

| | | Fold |
|---|---|---|
| Gene ID | Score(d) | Change |
| 112700 \|\| NRGN \|\| neurogranin (protein kinase C substrate, RC3) \|\| Hs.232004 \|\| | 5.868570153 | 29.54055 |
| H49511 \|\|4900 | | |
| 106815 \|\| MCOLN3 \|\| mucolipin 3 \|\| Hs.49344 \|\| AA171718 \|\| \|\| 55283□ | 5.614663369 | 17.06663 |
| 221573 \|\| NAB1 \|\| NGFI-A binding protein 1 (EGR1 binding protein 1) \|\| Hs.107474 \|\| | 5.458180498 | 13.67425 |
| N91896 \|\| 4664 | | |
| 115538 \|\| CDH24 \|\| cadherin-like 24 \|\| Hs.155912 \|\| AI668564 \|\| \|\| 64403□ | 5.423064855 | 13.38053 |
| 315169 \|\| R38064\| | 5.244632964 | 14.18661 |
| 116005 \|\| FAM13A1 \|\| family with sequence similarity 13, member A1 \|\| Hs.442818 \|\| | 5.228772563 | 11.29710 |
| AA024827 \|\| \|\| 10144□ | | |
| 100151 \|\| LIFR \|\| leukemia inhibitory factor receptor \|\| Hs.446501 \|\| H10192 \|\| \|\| 3977□ | 5.186844778 | 16.15460 |
| 117785 \|\| KIAA0182 \|\| KIAA0182 protein \|\| Hs.222171 \|\| H05099 \|\| \|\| 23199□ | 5.106082478 | 13.74697 |
| 228982 \|\| SYT7 \|\| synaptotagmin VII \|\| Hs.27495 \|\| R71085 \|\| \|\| 9066□ | 4.954662563 | 18.47525 |
| 107229 \|\| TRIP6 \|\| **thyroid hormone receptor interactor 6 \|\| Hs.380230 \|\| AA037466 \|\| \|\| | 4.939259153 | 9.94770 |
| 7205□ | | |
| 224110 \|\| RC3 \|\| rabconnectin-3 \|\| Hs.200828 \|\| R12847 \|\| \|\| 23312□ | 4.809513046 | 13.18260 |
| 118798 \|\| ALDH1A1 \|\| aldehyde dehydrogenase 1 family, member A1 \|\| Hs.76392 \|\| | 4.801627292 | 9.92152 |
| AA664101 \|\| \|\| 216□ | | |
| 220471 \|\| SYT7 \|\| synaptotagmin VII \|\| Hs.27495 \|\| H46001 \|\| \|\| 9066□ | 4.771508775 | 15.38155 |
| 113251 \|\| COL4A5 \|\| collagen, type IV, alpha 5 (Alport syndrome) \|\| Hs.169825 \|\| | 4.612516018 | 9.27510 |
| AA029107 \|\| \|\| 1287□ | | |
| 113063 \|\| NR3C1 \|\| nuclear receptor subfamily 3, group C, member 1 (glucocorticoid | 4.588166816 | 7.69559 |
| receptor) \|\| Hs.126608 \|\| N30428 \|\| Glucocorticoid receptor \|\| 2908□ | | |
| 109340 \|\| FKBP5 \|\| FK506 binding protein 5 \|\| Hs.7557 \|\| AA872767 \|\| \|\| 2289□ | 4.575877554 | 20.12744 |
| 99942 \|\| LOC112476 \|\| similar to lymphocyte antigen 6 complex, locus G5B; G5b | 4.508258098 | 10.21270 |
| protein; open reading frame 31 \|\| Hs.23650 \|\| AA398262 \|\| \|\| 112476□ | | |
| 99923 \|\| CDH24 \|\| cadherin-like 24 \|\| Hs.155912 \|\| AI732266 \|\| \|\| 64403□ | 4.500829445 | 11.98223 |
| 104660 \|\| ATRNL1 \|\| attractin-like 1 \|\| Hs.407474 \|\| H11917 \|\| \|\| 26033□ | 4.472527012 | 10.20115 |
| 98405 \|\| GPM6B glycoprotein M6B \|\| Hs.5422 \|\| AA284329 \|\| \|\| 2824□ | 4.327796311 | 7.35239 |
| 110842 \|\| SSX2IP \|\| synovial sarcoma, X breakpoint 2 interacting protein \|\| Hs.22587 \|\| | 4.222276734 | 6.93002 |
| H20847 \|\| \|\| 117178□ | | |
| 110377 \|\| CBLN2 \|\| cerebellin 2 precursor \|\| Hs.131596 \|\| H05818 \|\| \|\| 147381□ | 4.208342686 | 7.66680 |
| 102657 \|\| ITPR2 \|\| **inositol 1,4,5-triphosphate receptor, type 2 \|\| Hs.512235 \|\| R68020 \|\| | 4.205437 | 8.15323 |
| \|\| 3709□ | | |
| 220143 \|\| PROC \|\| protein C (inactivator of coagulation factors Va and VIIIa) \|\| Hs.2351 \|\| | 4.168474337 | 8.22620 |
| AI021885 \|\| \|\| 5624□ | | |
| 221690 \|\| SSX2IP \|\| synovial sarcoma, X breakpoint 2 interacting protein \|\| Hs.22587 \|\| | 4.143906703 | 7.08362 |
| AA909354 \|\| \|\| 117178□ | | |
| 308435 \|\| ASS \|\| argininosuccinate synthetase \|\| Hs.160786 \|\| AA676405 \|\| \|\| 445□ | 4.121733166 | 12.42701 |
| 117499 \|\| CDC42EP4 \|\| CDC42 effector protein (Rho GTPase binding) 4 \|\| Hs.3903 \|\| | 4.104416144 | 6.60574 |
| W32509 \|\| \|\| 23580□ | | |
| 99977 \|\| LIFR \|\| leukemia inhibitory factor receptor \|\| Hs.446501 \|\| AA131466 \|\| \|\| 3977□ | 4.082142767 | 7.82320 |
| 115590 \|\| STAT6 \|\| signal transducer and activator of transcription 6, interleukin-4 | 4.074026703 | 8.39293 |
| induced \|\| Hs.437475 \|\| T72202 \|\| \|\| 6778□ | | |
| 111666 \|\| BHLHB3 \|\| basic helix-loop-helix domain containing, class B, 3 \|\| Hs.437282 \|\| | 4.070419683 | 7.42424 |
| AA164818 \|\| \|\| 79365□ | | |
| 98603 \|\| \|\| \|\| \|\| W52112 \|\| \|\| □ | 4.03330383 | 6.51654 |
| 161377 \|\| PRKCD \|\| protein kinase C, delta \|\| Hs.155342 \|\| AA005214 \|\| PKC | 4.0244938 | 8.38109 |
| delta = Protein kinase C, delta \|\| 5580□ | | |
| 104837 \|\| HIST1H2AC \|\| histone 1, H2ac \|\| Hs.28777 \|\| AA452933 \|\| \|\| 8334□ | 3.999960465 | 7.60844 |
| 101789 \|\| \|\| Transcribed sequences \|\| Hs.527552 \|\| AA454562 \|\| \|\| □ | 3.986592961 | 12.47434 |
| 224472 \|\| \|\| Transcribed sequence with strong similarity to protein sp: P00722 (E. coli) | 3.975340688 | 6.88518 |
| BGAL_ECOLI Beta-galactosidase \|\| Hs.387246 \|\| AA705029 \|\| \|\| □ | | |
| 98640 \|\| CART1 \|\| **cartilage paired-class homeoprotein 1 \|\| Hs.41683 \|\| AA418020 \|\| \|\| | 3.974968121 | 11.13968 |
| 8092□ | | |
| 111056 \|\| TRA@ \|\| T cell receptor alpha locus \|\| Hs.74647 \|\| AA427491 \|\| \|\| 6955□ | 3.955502475 | 9.21489 |
| 117507 \|\| MSI2 \|\| musashi homolog 2 (Drosophila) \|\| Hs.185084 \|\| N45139 \|\| \|\| 124540□ | 3.947137141 | 6.34876 |
| 117186 \|\| ASP \|\| AKAP-associated sperm protein \|\| Hs.381089 \|\| AA453616 \|\| \|\| 83853□ | 3.929273199 | 19.75772 |
| 110941 \|\| ASS \|\| argininosuccinate synthetase \|\| Hs.160786 \|\| AA676466 \|\| \|\| 445□ | 3.896392778 | 11.12526 |
| 109657 \|\| SSX2IP \|\| synovial sarcoma, X breakpoint 2 interacting protein \|\| Hs.22587 \|\| | 3.88749883 | 5.66424 |
| H08594 \|\| \|\| 117178□ | | |
| 102887 \|\| KIAA0182 \|\| KIAA0182 protein \|\| Hs.222171 \|\| AI023801 \|\| \|\| 23199□ | 3.879701897 | 8.04762 |
| 107561 \|\| NFIX \|\| nuclear factor I/X (CCAAT-binding transcription factor) \|\| Hs.35841 \|\| | 3.875579115 | 6.54567 |
| R19306 \|\| \|\| 4784□ | | |
| 103020 \|\| GPM6B \|\| glycoprotein M6B \|\| Hs.5422 \|\| R42852 \|\| \|\| 2824□ | 3.841170241 | 5.97714 |
| 309960 \|\| ARHGAP25 \|\| Rho GTPase activating protein 25 \|\| Hs.1528 \|\| AI264291 \|\| \|\| | 3.831297695 | 6.72361 |
| 9938□ | | |
| 107869 \|\| GABRA2 \|\| gamma-aminobutyric acid (GABA) A receptor, alpha 2 \|\| Hs.91343 | 3.819700648 | 11.75424 |
| \|\| R12710 \|\| \|\| 2555□ | | |
| 108977 \|\| ALPI \|\| alkaline phosphatase, intestinal \|\| Hs.37009 \|\| AA190871 \|\| \|\| 248□ | 3.800425885 | 8.70847 |
| 116929 \|\| SORBS1 \|\| sorbin and SH3 domain containing 1 \|\| Hs.108924 \|\| AA459944 \|\| | 3.793310062 | 6.64177 |
| \|\| 10580□ | | |
| 102937 \|\| DDR1 \|\| discoidin domain receptor family, member 1 \|\| Hs.423573 \|\| H41900 \|\| | 3.785185886 | 8.51211 |
| \|\| 780□ | | |
| 105491 \|\| CDC42EP4 \|\| CDC42 effector protein (Rho GTPase binding) 4 \|\| Hs.3903 \|\| | 3.72386013 | 5.47888 |
| AA449773 \|\| \|\| 23580□ | | |
| 116828 \|\| DPYD \|\| dihydropyrimidine dehydrogenase \|\| Hs.1602 \|\| AA428170 \|\| | 3.631509152 | 5.37965 |
| Dihydropyrimidine dehydrogenase \|\| 1806□ | | |
| 315947 \|\| TRERF1 \|\| transcriptional regulating factor 1 \|\| Hs.50102 \|\| AI299636 \|\| \|\| | 3.606443217 | 7.12071 |
| 55809□ | | |
| 120943 \|\| PRKCD \|\| protein kinase C, delta \|\| Hs.155342 \|\| H11054 \|\| \|\| 5580□ | 3.578206246 | 7.54485 |
| 223681 \|\| HIST1H2BD \|\| histone 1, H2bd \|\| Hs.180779 \|\| AA885642 \|\| \|\| 3017□ | 3.560535595 | 9.28950 |
| 114324 \|\| DKFZP586A0522 \|\| DKFZP586A0522 protein \|\| Hs.288771 \|\| T49984 \|\| \|\| | 3.443699097 | 5.55837 |
| 25840□ | | |
| 222016 \|\| DUSP22 \|\| dual specificity phosphatase 22 \|\| Hs.29106 \|\| H42417 \|\| \|\| 56940□ | 3.433107171 | 5.68075 |
| 316002 \|\| TLE3 \|\| transducin-like enhancer of split 3 (E(sp1) homolog, Drosophila) \|\| | 3.415630959 | 6.00721 |
| Hs.287362 \|\| AI216623 \|\| \|\| 7090□ | | |
| 118264 \|\| KBTBD2 \|\| **kelch repeat and BTB (POZ) domain containing 2 \|\| Hs.20237 \|\| | 3.380904147 | 4.88429 |
| AA418251 \|\| \|\| 25948□ | | |
| 313410 \|\| AK3 \|\| adenylate kinase 3 \|\| Hs.10862 \|\| AA947132 \|\| \|\| 205□ | 3.379722534 | 5.29497 |
| 109848 \|\| BCL2 \|\| B-cell CLL/lymphoma 2 \|\| Hs.79241 \|\| W61100 \|\| BCL-2 \|\| 596□ | 3.377481632 | 4.62286 |
| 106544 \|\| SHMT2 \|\| **serine hydroxymethyltransferase 2 (mitochondrial) \|\| Hs.75069 \|\| | 3.365132308 | 5.09893 |
| N67017 \|\| \|\| 6472□ | | |
| 103825 \|\| \|\| \|\| \|\| W86653 \|\| \|\| □ | 3.364772832 | 7.67650 |
| 114436 \|\| CYP3A5 \|\| cytochrome P450, family 3, subfamily A, polypeptide 5 \|\| | 3.363416052 | 6.45275 |
| Hs.150276 \|\| AA873089 \|\| 1577□ | | |
| 112324 \|\| COL17A1 \|\| collagen, type XVII, alpha 1 \|\| Hs.117938 \|\| H87535 \|\| \|\| 1308□ | 3.329744105 | 12.84318 |
| 106244 \|\| ENC1 \|\| ectodermal-neural cortex (with BTB-like domain) \|\| Hs.104925 \|\| | 3.32317539 | 8.56967 |
| AA100036 \|\| Pig10 = p53-inducible gene = Similar to actin-binding protein EN \|\| 8507□ | | |
| 113626 \|\| NFIB \|\| nuclear factor I/B \|\| Hs.302690 \|\| R51361 \|\| \|\| 4781□ | 3.31848291 | 6.01142 |
| 117523 \|\| BTEB1 \|\| basic transcription element binding protein 1 \|\| Hs.150557 \|\| N80235 | 3.30793378 | 4.73192 |
| \|\| \|\| 687□ | | |
| 111246 \|\| PCSK2 \|\| proprotein convertase subtilisin/kexin type 2 \|\| Hs.315186 \|\| R37959 | 3.303352118 | 9.29640 |
| \|\| \|\| 5126□ | | |
| 223489 \|\| CLIC6 \|\| chloride intracellular channel 6 \|\| Hs.353146 \|\| AA630241 \|\| \|\| | 3.301789075 | 5.58281 |
| 54102□ | | |
| 118058 \|\| HIST2H2BE \|\| histone 2, H2be \|\| Hs.2178 \|\| AA010223 \|\| \|\| 8349□ | 3.29466814 | 6.40681 |
| 106359 \|\| CYGB \|\| cytoglobin \|\| Hs.95120 \|\| AA025407 \|\| \|\| 114757□ | 3.287522707 | 6.22708 |
| 101148 \|\| ARHI \|\| ras homolog gene family, member I \|\| Hs.194695 \|\| W72033 \|\| \|\| | 3.24685291 | 5.72150 |
| 9077□ | | |
| 103772 \|\| FLJ21069 \|\| hypothetical protein FLJ21069 \|\| Hs.341806 \|\| R24223 \|\| \|\| | 3.246601639 | 4.86146 |
| 79745□ | | |
| 114192 \|\| TM4SF2 \|\| transmembrane 4 superfamily member 2 \|\| Hs.439586 \|\| N93505 \|\| | 3.239809005 | 6.64422 |
| TALLA = T-cell acute lymphoblastic leukemia associated antigen \|\| 7102□ | | |
| 308977 \|\| F3 \|\| coagulation factor III (thromboplastin, tissue factor) \|\| Hs.62192 \|\| | 3.233349397 | 5.75669 |
| AI313387 \|\| \|\| 2152□ | | |
| 111924 \|\| \|\| CDNA clone MGC: 52263 IMAGE: 4123447, complete cds \|\| Hs.251664 \|\| | 3.232700101 | 5.86456 |
| H23457 \|\| \|\| □ | | |
| 102295 \|\| FNTA \|\| farnesyltransferase, CAAX box, alpha \|\| Hs.356463 \|\| N78902 \|\| \|\| | 3.207096922 | 5.68556 |
| 2339□ | | |
| 107638 \|\| SPOCK3 \|\| sparc/osteonectin, cwcv and kazal-like domains proteoglycan | 3.200688916 | 13.58934 |
| (testican) 3 \|\| Hs.159425 \|\| AA199586 \|\| \|\| 50859□ | | |
| 108672 \|\| GLI2 \|\|GLI-Kruppel family member GLI2 \|\| Hs.111867 \|\| AI822076 \|\| \|\| 2736□ | 3.185700834 | 4.47430 |
| 224819 \|\| VAMP5 \|\| vesicle-associated membrane protein 5 (myobrevin) \|\| Hs.172684 \|\| | 3.158207782 | 4.86338 |
| T62963 \|\| \|\| 10791□ | | |
| 117183 \|\| TLE3 \|\|transducin-like enhancer of split 3 (E(sp1) homolog, Drosophila) \|\| | 3.148770476 | 5.06080 |
| Hs.287362 \|\| AA136692 \|\| \|\| 70905□ | | |
| 100793 \|\| DDR1 \|\| discoidin domain receptor family, member 1 \|\| Hs.423573 \|\| | 3.145484603 | 6.26005 |
| AA400885 \|\| \|\| 780□ | | |
| 120397 \|\| DUSP22 \|\| dual specificity phosphatase 22 \|\| Hs.29106 \|\| H91044 \|\| \|\| 56940□ | 3.132220782 | 4.43448 |
| 118047 \|\| FLJ39155 \|\| hypothetical protein FLJ39155 \|\| Hs.20103 \|\| AA989292 \|\| \|\| | 3.109303101 | 4.78287 |
| 133584□ | | |
| 104357 \|\| HIST1H2BD \|\| histone 1, H2bd \|\| Hs.180779 \|\| N33927 \|\| \|\| 3017□ | 3.103151527 | 7.09773 |
| 221733 \|\| HIST1H2BG \|\| histone 1, H2bg \|\| Hs.182137 \|\| R98472 \|\| \|\| 8339□ | 3.096820366 | 7.17192 |
| 107094 \|\| \|\| Transcribed sequences \|\| Hs.444291 \|\| AI004175 \|\| \|\| □ | 3.095372642 | 5.05888 |
| 100858 \|\| GSN \|\| gelsolin (amyloidosis, Finnish type) \|\| Hs.446537 \|\| T98455 \|\| \|\| 2934□ | 3.088653073 | 4.81433 |
| 307599 \|\| HOXB6 \|\| homeo box B6 \|\| Hs.147465 \|\| AA918749 \|\| \|\| 3216□ | 3.081546257 | 4.09734 |
| 106155 \|\| \|\| \|\| \|\| R00822 \|\| \|\| □ | 3.032036232 | 10.73946 |
| 120725 \|\| DC36 \|\| **COBW-like placental protein \|\| Hs.355950 \|\| AA485713 \|\| \|\| | 2.998298522 | 4.95397 |
| 389760□ | | |
| 103560 \|\| DKFZP586A0522 \|\| DKFZP586A0522 protein \|\| Hs.288771 \|\| N70948 \|\| \|\| | 2.9681905553 | 4.39226 |
| 25840□ | | |
| 103727 \|\| GPM6B \|\| glycoprotein M6B \|\| Hs.5422 \|\| N59368 \|\| \|\| 2824□ | 2.94976801 | 3.69073 |
| 118309 \|\| KIAA0089 \|\| KIAA0089 protein \|\| Hs.82432 \|\| AA485401 \|\| \|\| 23171□ | 2.942166449 | 4.90425 |
| 110980 \|\| BCL2 \|\| B-cell CLL/lymphoma 2 \|\| Hs.79241 \|\| H73130 \|\| BCL-2 \|\| 596□ | 2.934468028 | 5.09656 |
| 101323 \|\| ENC1 \|\| ectodermal-neural cortex (with BTB-like domain) \|\| Hs.104925 \|\| | 2.925636315 | 9.23060 |
| H72122 \|\| \|\| 8507□ | | |
| 109105 \|\| LGALS3BP \|\| lectin, galactoside-binding, soluble, 3 binding protein \|\| Hs.79339 | 2.92448378 | 3.99258 |
| \|\| AA485353 \|\| \|\| 3959□ | | |
| 114497 \|\| TGFB1I4 \|\| transforming growth factor beta 1 induced transcript 4 \|\| Hs.114360 | 2.918570971 | 5.77290 |
| \|\| AA664389 \|\| \|\| 8848□ | | |
| 100450 \|\| PRKCD \|\| protein kinase C, delta \|\| Hs.155342 \|\| AA496360 \|\| \|\| 5580□ | 2.917370288 | 4.39408 |
| 118803 \|\| ITM2A \|\| integral membrane protein 2A \|\| Hs.17109 \|\| N72450 \|\| \|\| 9452□ | 2.905988173 | 4.80072 |
| 101836 \|\| CEP1 \|\| centrosomal protein 1 \|\| Hs.246344 \|\| H66030 \|\| \|\| 11064□ | 2.89872683 | 4.22671 |
| 114678 \|\| SCNN1A \|\| sodium channel, nonvoltage-gated 1 alpha \|\| Hs.446415 \|\| | 2.898667699 | 9.34777 |
| AA458982 \|\| \|\| 6337□ | | |
| 220092 \|\| LOC220594 \|\| TL132 protein \|\| Hs.234573 \|\| AA521366 \|\| \|\| 220594□ | 2.872830834 | 5.70051 |
| 102512 \|\| NCOA2 \|\| nuclear receptor coactivator 2 \|\| Hs.446678 \|\| R77770 \|\| \|\| 10499□ | 2.868117597 | 3.83819 |
| 118118 \|\| BTG2 \|\| BTG family, member 2 \|\| Hs.75462 \|\| H69582 \|\| \|\| 7832□ | 2.828669849 | 4.28081 |
| 221400 \|\| USP6 \|\| **ubiquitin specific protease 6 (Tre-2 oncogene) \|\| Hs.448851 \|\| | 2.823580558 | 5.80315 |
| N92188 \|\| \|\| 9098□ | | |
| 103266 \|\| USP32 \|\| ubiquitin specific protease 32 \|\| Hs.436133 \|\| AA434005 \|\| \|\| 84669□ | 2.819656885 | 5.69285 |
| 106357 \|\| LMO7 \|\| LIM domain only 7 \|\| Hs.5978 \|\| H22825 \|\| \|\| 4008□ | 2.799961253 | 4.00002 |
| 308400 \|\| TGFB1I4 \|\| transforming growth factor beta 1 induced transcript 4 \|\| Hs.114360 | 2.798287358 | 5.52658 |
| \|\| AA398237 \|\| \|\| 8848□ | | |
| 110057 \|\| KRT8 \|\| keratin 8 \|\| Hs.356123 \|\| AA598517 \|\| \|\| 3856□ | 2.795633119 | 5.84020 |
| 114175 \|\| PCSK2 \|\| proprotein convertase subtilisin/kexin type 2 \|\| Hs.315186 \|\| | 2.76479657 | 7.29683 |
| AA069517 \|\| \|\| 5126□ | | |
| 109207 \|\| ZNF542 \|\| zinc finger protein 542 \|\| Hs.406330 \|\| N50864 \|\| \|\| 147947□ | 2.760952386 | 4.30734 |
| 99151 \|\| ARHGAP6 \|\| Rho GTPase activating protein 6 \|\| Hs.250830 \|\| AA425035 \|\| \|\| | 2.728831628 | 5.21909 |
| 395□ | | |
| 113510 \|\| TGM2 \|\| **transglutaminase 2 (C polypeptide, protein-glutamine-gamma- | 2.714872308 | 5.33962 |
| glutamyltransferase) \|\| Hs.512708 \|\| R97066 \|\| \|\| 7052□ | | |
| 104093 \|\| LSP1 \|\| lymphocyte-specific protein 1 \|\| Hs.56729 \|\| T83159 \|\| \|\| 4046□ | 2.713163327 | 3.13833 |
| 111849 \|\| TXNIP \|\| **thioredoxin interacting protein \|\| Hs.179526 \|\| AA873257 \|\| \|\| | 2.709255953 | 7.44460 |
| 10628□ | | |
| 117912 \|\| \|\| Clone IMAGE: 4794726, mRNA \|\| Hs.367688 \|\| AA127909 \|\| \|\| □ | 2.707861668 | 3.52067 |
| 100270 \|\| NRG2 \|\| neuregulin 2 \|\| Hs.408515 \|\| AA706226 \|\| \|\| 9542□ | 2.706092477 | 4.66884 |
| 222426 \|\| PEPP2 \|\| phosphoinositol 3-phosphate-binding protein-2 \|\| Hs.242537 \|\| | 2.692206447 | 4.80628 |
| R53323 \|\| \|\| 54477□ | | |
| 105455 \|\| \|\| \|\| \|\| R41972 \|\| \|\| □ | 2.689285073 | 2.94017 |
| 112361 \|\| \|\| \|\| \|\| R88878 \|\| \|\| □ | 2.687589882 | 3.98039 |
| 115923 \|\| HIST1H2BK \|\| histone 1, H2bk \|\| Hs.247817 \|\| N71982 \|\| \|\| 85236□ | 2.677065771 | 4.28699 |
| 110633 \|\| \|\| Transcribed sequences \|\| Hs.436897 \|\| AA894559 \|\| \|\| □ | 2.666294299 | 4.12398 |
| 102010 \|\| TNNT3 \|\| troponin T3, skeletal, fast \|\| Hs.73454 \|\| AA449931 \|\| \|\| 7140□ | 2.653841359 | 4.39848 |
| 308512 \|\| \|\| Transcribed sequence with strong similarity to protein ref: NP_000041.1 | 2.651043066 | 4.44397 |
| (H. sapiens) argininosuccinate synthetase [Homo sapiens] \|\| Hs.368019 \|\| AA995233 \|\| \|\| □ | | |
| 223144 \|\| USP53 \|\| ubiquitin specific protease 53 \|\| Hs.135457 \|\| AA504253 \|\| \|\| 545325 | 2.638244845 | 7.32451 |
| 100398 \|\| PRKACB \|\| protein kinase, cAMP-dependent, catalytic, beta \|\| Hs.156324 \|\| | 2.635802344 | 6.50469 |
| AA018979 \|\| PKA-C-BETA = cAMP-dependent protein kinase, BETA-catalytic sub \|\| | | |
| 5567□ | | |
| 223072 \|\| TOX \|\| thymus high mobility group box protein TOX \|\| Hs.439767 \|\| R39310 \|\| | 2.633538309 | 6.90145 |
| \|\| 9760□ | | |
| 99715 \|\| OR2B6 \|\| **olfactory receptor, family 2, subfamily B, member 6 \|\| Hs.532145 \|\| | 2.608000719 | 4.95597 |
| N91194 \|\| \|\| 26212□ | | |
| 111499 \|\| \|\| \|\| \|\| R51382 \|\| \|\| □ | 2.604286987 | 3.49178 |
| 224251 \|\| \|\| \|\| \|\| AA858394 \|\| \|\| □ | 2.603044714 | 4.19482 |
| 116189 \|\| GASP \|\| G protein-coupled receptor-associated sorting protein \|\| Hs.113082 \|\| | 2.593039634 | 4.64358 |
| AA702949 \|\| \|\| 9737□ | | |
| 185134 \|\| MGC71745 \|\| similar to embigin \|\| Hs.528324 \|\| AA521108 \|\| glycogenin-2 like | 2.589900132 | 4.32821 |
| mRNA \|\| 133418□ | | |
| 113723 \|\| TTYH2 \|\| tweety homolog 2 (Drosophila) \|\| Hs.27935 \|\| AA434395 \|\| \|\| 94015□ | 2.573660123 | 3.86906 |
| 221431 \|\| KIAA1946 \|\| KIAA1946 \|\| Hs.172792 \|\| AA663361 \|\| \|\| 165215□ | 2.568980108 | 4.27931 |
| 1045520 \|\| \|\| \|\| \|\| T69767 \|\| \|\| □ | 2.556699809 | 3.60935 |
| 104008 \|\| CEACAM1 \|\| carcinoembryonic antigen-related cell adhesion molecule 1 | 2.538451059 | 3.35845 |
| (biliary glycoprotein) \|\| Hs.512682 \|\| AA406571 \|\| \|\| 634□ | | |
| 106702 \|\| EFA6R \|\| ADP-ribosylation factor guanine nucleotide factor 6 \|\| Hs.408177 \|\| | 2.526955395 | 4.71478 |
| AA232926 \|\| \|\| 23362□ | | |
| 116766 \|\| CAMK1 \|\| calcium/calmodulin-dependent protein \|\| kinase I \|\| Hs.512804 \|\| | 2.513439708 | 3.66943 |
| H29322 \|\| cam kinase I \|\| 8536□ | | |
| 110550 \|\| SSX2IP \|\| synovial sarcoma, X breakpoint 2 interacting protein Hs.22587 \|\| | 2.499526726 | 3.52006 |
| R24450 \|\| \|\| 117178□ | | |
| 316392 \|\| \|\| Human S6 H-8 mRNA expressed in chromosome 6-suppressed melanoma | 2.495505075 | 4.01719 |
| cells. \|\| Hs.446408 \|\| AI276270 \|\| \|\| □ | | |
| 114425 \|\| COL21A1 \|\| collagen, type XXI, alpha 1 \|\| Hs.408757 \|\| W92416 \|\| \|\| 81578□ | 2.472430303 | 4.24156 |
| 224002 \|\| SPAG5 \|\| sperm associated antigen 5 \|\| Hs.16244 \|\| T97349 \|\| \|\| 10615□ | 2.452370351 | 4.73947 |
| 117871 \|\| ITM2A \|\| integral membrane protein 2A \|\| Hs.17109 \|\| AA775257 \|\| \|\| 9452□ | 2.437898136 | 4.50861 |
| 100791 \|\| SLC40A1 \|\| **solute carrier family 40 (iron-regulated transporter), member 1 \|\| | 2.422850413 | 4.35197 |
| Hs.409875 \|\| AA417956 \|\| \|\| 30061□ | | |
| 110707 \|\| \|\| Similar to RIKEN cDNA 6530401L14 gene (LOC400342), mRNA \|\| | 2.416633883 | 4.53746 |
| Hs.444306 \|\| N63575 \|\| \|\| 400342□ | | |
| 330247 \|\| LOC90355 \|\| hypothetical gene supported by AF038182; BC009203 \|\| | 2.413773554 | 3.68080 |
| Hs.25925 \|\| R52538 \|\| \|\| 90355□ | | |
| 113495 \|\| \|\| Colorectal cancer-related mRNA sequence \|\| Hs.436383 \|\| R42331 \|\| \|\| □ | 2.404710156 | 3.76478 |
| 110439 \|\| TXNIP \|\| thioredoxin interacting protein \|\| Hs.179526 \|\| AA044633 \|\| brain- | 2.393622379 | 5.63789 |
| expressed HHCPA78 homolog = Induced in HL60 cells treate \|\| 10628□ | | |
| 309717 \|\| IGF1 \|\| insulin-like growth factor 1 (somatomedin C) \|\| Hs.308053 \|\| N67876 \|\| | 2.387514556 | 4.38595 |
| \|\| 3479□ | | |
| 103796 \|\| TXNIP \|\| **thioredoxin interacting protein \|\| Hs.179526 \|\| AA931478 \|\| \|\| | 2.36442546 | 4.88547 |
| 10628□ | | |
| 225252 \|\| DKFZP586A0522 \|\| DKFZP586A0522 protein \|\| Hs.288771 \|\| AA704713 \|\| \|\| | 2.362275057 | 2.59570 |
| 25840□ | | |
| 107656 \|\| ANK3 \|\| ankyrin 3, node of Ranvier (ankyrin G) \|\| Hs.440478 \|\| AA677185 \|\| \|\| | 2.342560492 | 4.30513 |
| 288□ | | |
| 115961 \|\| KIAA0746 \|\| KIAA0746 protein \|\| Hs.49500 \|\| AA456569 \|\| \|\| 23231□ | 2.338584049 | 2.95755 |
| 113314 \|\| NFIB \|\| nuclear factor I/B \|\| Hs.302690 \|\| AA455935 \|\| \|\| 4781□ | 2.332172911 | 4.32609 |
| 108053 \|\| FLJ32810 \|\| hypothetical protein FLJ32810 \|\| Hs.23193 \|\| AA417982 \|\| \|\| | 2.328258458 | 4.11479 |
| 143872□ | | |
| 98934 \|\| IGF1 \|\| insulin-like growth factor 1 (somatomedin C) \|\| Hs.308053 \|\| AA456321 \|\| | 2.322639378 | 3.40672 |
| \|\| 3479□ | | |
| 119117 \|\| C4A \|\| complement component 4A \|\| Hs.150833 \|\| AA664406 \|\| \|\| 720□ | 2.318517111 | 4.38676 |
| 307742 \|\| FZD7 \|\| frizzled homolog 7 (Drosophila) \|\| Hs.173859 \|\| H71474 \|\| \|\| 8324□ | 2.317658512 | 3.39264 |
| 178306 \|\| LCN2 \|\| lipocalin 2 (oncogene 24p3) \|\| Hs.204238 \|\| N79823 \|\| Neutrophil | 2.314137084 | 3.28794 |
| gelatinase-associated lipocalin \|\| 3934□ | | |
| 113989 \|\| HPS1 \|\| Hermansky-Pudlak syndrome 1 \|\| Hs.404568 \|\| AA418683 \|\| \|\| 3257□ | 2.276418398 | 2.87892 |
| 103650 FOXG1B \|\| forkhead box G1B \|\| Hs.386249 \|\| R19033 \|\| \|\| 2290□ | 2.265121646 | 5.33552 |
| 101175 \|\| MPEG1 \|\| macrophage expressed gene 1 \|\| Hs.62264 \|\| AA487527 \|\| \|\| | 2.258920444 | 2.76491 |
| 219972□ | | |
| 107729 \|\| ASNS \|\| asparagine synthetase \|\| Hs.446546 \|\| AA894927 \|\| \|\| 440□ | 2.256748069 | 2.58639 |
| 101795 \|\| RQCD1 \|\| RCD1 required for cell differentiation1 homolog (S. pombe) \|\| | 2.254131813 | 3.71520 |
| Hs.148767 \|\| H68272 \|\| \|\| 9125□ | | |
| 105536 \|\| FLJ10748 \|\| hypothetical protein FLJ10748 \|\| Hs.10414 \|\| AA293895 \|\| \|\| | 2.238067875 | 5.33672 |
| 55220□ | | |
| 108987 \|\| NAB1 \|\| NGFI-A binding protein 1 (EGR1 binding protein 1) \|\| Hs.107474 \|\| | 2.234951049 | 6.46312 |
| AA486027 \|\| \|\| 4664□ | | |
| 309045 \|\| \|\| \|\| \|\| AA953735 \|\| \|\| □ | 2.231248986 | 4.93358 |
| 104358 \|\| DKFZP586A0522 \|\| DKFZP586A0522 protein \|\| Hs.288771 \|\| R34273 \|\| \|\| | 2.220134947 | 2.59322 |
| 25840□ | | |
| 110016 \|\| MERTK \|\| c-mer proto-oncogene tyrosine kinase \|\| Hs.306178 \|\| AA436564 \|\| | 2.218715784 | 3.81665 |
| c-mer = cellular proto-oncogene \|\| 10461□ | | |
| 220107 \|\| \|\| CDNA FLJ30478 fis, clone BRAWH1000167 \|\| Hs.298258 \|\| R81987 \|\| \|\| □ | 2.206089072 | 3.78541 |
| 311366 \|\| \|\| \|\| \|\| AI268844 \|\| \|\| □ | 2.193999889 | 2.96716 |
| 102776 \|\| TMEM30B \|\| transmembrane protein 30B \|\| Hs.146180 \|\| W39430 \|\| \|\| | 2.18456988 | 4.86391 |
| 161291□ | | |
| 117055 \|\| \|\| Transcribed sequences \|\| Hs.419768 \|\| R99739 \|\| \|\| □ | 2.176468407 | 3.72613 |
| 110150 \|\| HSD11B1 \|\| hydroxysteroid (11-beta) dehydrogenase 1 \|\| Hs.275215 \|\| | 2.129719548 | 2.90169 |
| AA150918 \|\| \|\| 3290□ | | |
| 316150 \|\| KIAA0605 \|\| KIAA0605 gene product \|\| Hs.200594 \|\| AI014304 \|\| \|\| 9719□ | 2.11370019 | 4.01323 |
| 224951 \|\| DSIPI \|\| delta sleep inducing peptide, immunoreactor \|\| Hs.420569 \|\| | 2.109120643 | 3.12031 |
| AA490606 \|\| \|\| 1831□ | | |
| 311281 \|\| \|\| Transcribed sequence with moderate similarity to protein ref: NP_002433.1 | 2.086546918 | 2.08778 |
| (H. sapiens) Musashi \|\| Hs.156395 \|\| AI339225 \|\| \|\| □ | | |
| 107715 \|\| SFTPC \|\| surfactant, pulmonary-associated protein C \|\| Hs.1074 \|\| T39188 \|\| \|\| | 2.083322212 | 3.42885 |
| 6440□ | | |
| 119848 \|\| FLJ39155 \|\| hypothetical protein FLJ39155 \|\| Hs.20103 \|\| R08140 \|\| \|\| | 2.068567212 | 3.14793 |
| 133584□ | | |
| 308452 \|\| COL4A5 \|\| collagen, type IV, alpha 5 (Alport syndrome) \|\| Hs.169825 \|\| | 2.067198112 | 2.63036 |
| AA953254 \|\| \|\| 1287□ | | |
| 102660 \|\| APOD \|\| apolipoprotein D \|\| Hs.75736 \|\| H15842 \|\| \|\| 347□ | 2.06212122 | 1.32535 |
| 114673 \|\| \|\| \|\| \|\| AI668696 \|\| \|\| □ | 2.057990106 | 3.26355 |
| 106632 \|\| DKFZP564O0823 \|\| DKFZP564O0823 protein \|\| Hs.105460 \|\| AA086292 \|\| \|\| | 2.041455324 | 5.35698 |
| 25849□ | | |
| 116081 \|\| SPOCK \|\| sparc/osteonectin, cwcv and kazal-like domains proteoglycan | 2.032111934 | 3.05180 |
| (testican) \|\| Hs.93029 \|\| AA436142 \|\| \|\| 6695□ | | |
| 117274 \|\| CHRDL1 \|\| chordin-like 1 \|\| Hs.440324 \|\| AA040424 \|\| \|\| 91851□ | 2.028233518 | 3.81194 |
| 224412 \|\| \|\| Transcribed sequences \|\| Hs.374495 \|\| AA489040 \|\| \|\| □ | 2.007624423 | 2.71163 |
| 119319 \|\| ELF3 \|\| E74-like factor 3 (ets domain transcription factor, epithelial-specific) \|\| | 1.994663954 | 3.23887 |
| Hs.67928 \|\| AA433851 \|\| \|\| 1999□ | | |
| 112965 \|\| SLC40A1 \|\| solute carrier family 40 (iron-regulated transporter), member 1 \|\| | 1.992490711 | 3.35239 |
| Hs.409875 \|\| T52564 \|\| \|\| 30061□ | | |
| 307877 \|\| SSX2IP synovial sarcoma, X breakpoint 2 interacting protein \|\| Hs.22587 \|\| | 1.987341181 | 2.79462 |
| AA707473 \|\| \|\| 117178□ | | |
| 110563 \|\| GLI2 \|\| GLI-Kruppel family member GLI2 \|\| Hs.111867 \|\| H69695 \|\| \|\| 2736□ | 1.973683454 | 2.58628 |
| 306996 \|\| ZIC2 \|\| Zic family member 2 (odd-paired homolog, Drosophila) \|\| Hs.369063 \|\| | 1.964069627 | 10.21151 |
| AA988219 \|\| \|\| 7546□ | | |
| 109859 \|\| GABRA2 \|\| gamma-aminobutyric acid (GABA) A receptor, alpha 2 \|\| Hs.91343 | 1.95981482 | 2.56545 |
| \|\| N46354 \|\| \|\| 2555□ | | |
| 108178 \|\| SESN3 \|\| sestrin 3 \|\| Hs.271953 \|\| W95428 \|\| \|\| 143686□ | 1.950159056 | 3.43237 |
| 309686 \|\| \|\| \|\| \|\| AA215397 \|\| \|\| □ | 1.941192585 | 3.33314 |
| 107627 \|\| BCHE \|\| butyrylcholinesterase \|\| Hs.422857 \|\| AA885311 \|\| \|\| 590□ | 1.934965034 | 8.25846 |
| 113361 \|\| NFIB \|\| nuclear factor I/B \|\| Hs.302690 \|\| N77345 \|\| \|\| 4781□ | 1.926831695 | 2.98026 |
| 101547 \|\| FGF7 \|\| fibroblast growth factor 7 (keratinocyte growth factor) \|\| Hs.433252 \|\| | 1.923480666 | 2.54340 |
| AA009608 \|\| \|\| 2252□ | | |
| 307221 \|\| HIST1H2BJ \|\| histone 1, H2bj \|\| Hs.519945 \|\| AI076718 \|\| \|\| 8970□ | 1.91788819 | 2.65804 |
| 120698 \|\| LRAP \|\| leukocyte-derived arginine aminopeptidase \|\| Hs.374490 \|\| H17550 \|\| | 1.910714136 | 3.29774 |
| \|\| 64167□ | | |
| 308284 \|\| C1orf21 \|\| chromosome 1 open reading frame 21 \|\| Hs.12532 \|\| AA406569 \|\| \|\| | 1.895024416 | 2.44124 |
| 81563□ | | |
| 227041 \|\| ZNF542 \|\| zinc finger protein 542 \|\| Hs.406330 \|\| AA862414 \|\| \|\| 147947□ | 1.890460364 | 2.35758 |
| 101053 \|\| FOXD1 \|\| forkhead box D1 \|\| Hs.96028 \|\| AA069132 \|\| \|\| 2297□ | 1.882134303 | 2.61757 |
| 105351 \|\| PRKY \|\| protein kinase, Y-linked \|\| Hs.183165 \|\| N99154 \|\| \|\| 5616□ | 1.870918673 | 3.02860 |
| 104542 \|\| \|\| Transcribed sequence with moderate similarity to protein sp: P39195 | 1.869531547 | 2.94149 |
| (H. sapiens) ALU8_HUMAN Alu subfamily SX sequence contamination warning entry \|\| | | |
| Hs.136398 \|\| AA630663 \|\| \|\| □ | | |
| 111035 \|\| \|\| Clone IMAGE: 4794726, mRNA Hs.367688 \|\| R74357 \|\| \|\| □ | 1.869426041 | 2.15076 |
| 120978 \|\| GRIK2 \|\| **glutamate receptor, ionotropic, kainate 2 \|\| Hs.307494 \|\| H15417 \|\| | 1.868654578 | 3.49104 |
| \|\| 2898□ | | |
| 224053 \|\| NEFL \|\| neurofilament, light polypeptide 68 kDa \|\| Hs.107600 \|\| AA776041 \|\| \|\| | 1.846304265 | 3.93777 |
| 4747□ | | |
| 115391 \|\| PRPS2 \|\| phosphoribosyl pyrophosphate synthetase 2 \|\| Hs.104123 \|\| | 1.831840369 | 2.37407 |
| AA197344 \|\| \|\| 5634□ | | |
| 105718 \|\| KCNK2 \|\| potassium channel, subfamily K, member 2 \|\| Hs.202696 \|\| R59601 | 1.830896693 | 2.82252 |
| \|\| \|\| 3776□ | | |
| 118688 \|\| LIFR \|\| leukemia inhibitory factor receptor \|\| Hs.446501 \|\| AA115300 \|\| \|\| | 1.821137043 | 2.07404 |
| 3977□ | | |
| 120209 \|\| MSI2 \|\| musashi homolog 2 (Drosophila) \|\| Hs.185084 \|\| N52994 \|\| \|\| 124540□ | 1.814170817 | 1.71432 |
| 223240 \|\| PLEKHA4 \|\| pleckstrin homology domain containing, family A | 1.799925065 | 1.99714 |
| (phosphoinositide binding specific) member 4 \|\| Hs.9469 \|\| AA521373 \|\| \|\| 57664□ | | |
| 107930 \|\| SLC22A3 \|\| solute carrier family 22 (extraneuronal monoamine transporter), | 1.792495717 | 4.15013 |
| member 3 \|\| Hs.242721 \|\| R08120 \|\| \|\| 6581□ | | |
| 307082 \|\| \|\| Transcribed sequence with strong similarity to protein sp: P00722 (E. coli) | 1.778427606 | 3.26937 |
| BGAL_ECOLI Beta-galactosidase \|\| Hs.121518 \|\| AA976650 \|\| \|\| □ | | |
| 99803 \|\| PRKACB \|\| protein kinase, cAMP-dependent, catalytic, beta \|\| Hs.156324 \|\| | 1.771002017 | 3.93628 |
| AA459980 \|\| \|\| 5567□ | | |
| 104290 \|\| MYO9B \|\| myosin IXB \|\| Hs.159629 \|\| N51705 \|\| myosin-IXb \|\| 4650□ | 1.760367752 | 3.15721 |
| 224130 \|\| ACSL5 \|\| acyl-CoA synthetase long-chain family member 5 \|\| Hs.11638 \|\| | 1.733111026 | 1.86698 |
| AA705516 \|\| \|\| 51703□ | | |
| 107972 \|\| PPAP2B \|\| phosphatidic acid phosphatase type 2B \|\| Hs.432840 \|\| T71976 \|\| | 1.73238668 | 2.97845 |
| Dri42 = phosphatidic acid phosphohydrolase homolog \|\| 8613□ | | |
| 330675 \|\| AKR1C2 \|\| aldo-keto reductase family 1, member C2 (dihydrodiol | 1.732367275 | 3.05742 |
| dehydrogenase 2; bile acid binding protein; 3-alpha hydroxysteroid dehydrogenase, type | | |
| III) \|\| Hs.201967 \|\| AI924357 \|\| \|\| 1646□ | | |
| 225606 \|\| \|\| MRNA; cDNA DKFZp727C191 (from clone DKFZp727C191) \|\| Hs.131417 \|\| | 1.709821093 | 2.25989 |
| AA621408 \|\| \|\| □ | | |
| 312880 \|\| RGS2 \|\| regulator of G-protein signalling 2, 24 kDa \|\| Hs.78944 \|\| AI675670 \|\| \|\| | 1.702037886 | 2.85729 |
| 5997□ | | |
| 99120 \|\| UGCGL2 \|\| UDP-glucose ceramide glucosyltransferase-like 2 \|\| Hs.308242 \|\| | 1.686256625 | 2.45641 |
| AA424587 \|\| \|\| 55757□ | | |
| 226351 \|\| \|\| \|\| \|\| AA931758 \|\| \|\| □ | 1.682053223 | 2.44412 |
| 222092 \|\| \|\| \|\| \|\| T95670 \|\| \|\| □ | 1.678870234 | 1.85695 |
| 105040 \|\| SELL \|\| selectin L (lymphocyte adhesion molecule 1) \|\| Hs.82848 \|\| H00662 \|\| | 1.675994399 | 1.90404 |
| CD62L = L-selectin = LAM-1 = leukocyte adhesion molecule-1 \|\| 6402□ | | |
| 98991 \|\| ZC3HDC6 \|\| zinc finger CCCH type domain containing 6 \|\| Hs.190477 \|\| | 1.668801584 | 2.97735 |
| AA873249 \|\| \|\| 376940□ | | |
| 221145 \|\| \|\| \|\| \|\| AA699429 \|\| \|\| □ | 1.663264463 | 2.38839 |
| 102508 \|\| \|\| Transcribed sequences \|\| Hs.26331 \|\| H05961 \|\| \|\| □ | 1.610378507 | 1.91943 |
| 100751 \|\| EFNB1 \|\| ephrin-B1 \|\| Hs.144700 \|\| AA428778 \|\| \|\| 1947□ | 1.605968574 | 4.25159 |
| 110202 \|\| TPR \|\| **translocated promoter region (to activated MET oncogene) \|\| | 1.567502044 | 5.27651 |
| Hs.170472 \|\| AA451832 \|\| \|\| 7175□ | | |
| 119665 \|\| ERBB2 \|\| v-erb-b2 erythroblastic leukemia viral oncogene homolog 2, | 1.565105674 | 2.52312 |
| neuro/glioblastoma derived oncogene homolog (avian) \|\| Hs.446352 \|\| AA481939 \|\| \|\| | | |
| 2064□ | | |
| 113692 \|\| MGST1 \|\| microsomal glutathione S-transferase 1 \|\| Hs.389700 \|\| AA495935 \|\| | 1.535734648 | 1.72628 |
| Glutathione S-transferase, microsomal \|\| 4257□ | | |
| 101397 \|\| SAA1 \|\| serum amyloid A1 \|\| Hs.332053 \|\| H25546 \|\| \|\| 6288□ | 1.53510133 | 3.69876 |
| 98914 \|\| \|\| \|\| \|\| N95621 \|\| \|\| □ | 1.516072826 | 2.62381 |
| 99677 \|\| AKR1C1 \|\| aldo-keto reductase family 1, member C1 (dihydrodiol | 1.508661745 | 3.18648 |
| dehydrogenase 1; 20-alpha (3-alpha)-hydroxysteroid dehydrogenase) \|\| Hs.295131 \|\| | | |
| R93124 \|\| \|\| 1645□ | | |
| 99474 \|\| \|\| \|\| \|\| H80335 \|\| \|\| □ | 1.508273358 | 3.02897 |
| 220197 \|\| ATF5 \|\| activating transcription factors 5 \|\| Hs.9754 \|\| AA496253 \|\| \|\| 22809□ | 1.503638386 | 3.54907 |
| 226867 \|\| B3GTL \|\| beta 3-glycosyltransferase-like \|\| Hs.13205 \|\| N51325 \|\| \|\| 145173□ | 1.500340884 | 3.74207 |
| 111660 \|\| \|\| MRNA full length insert cDNA clone EUROIMAGE 248114 \|\| Hs.231971 \|\| | 1.497439793 | 2.96482 |
| AA424560 \|\| \|\| □ | | |
| 110252 \|\| SLC37A4 \|\| solute carrier family 37 (glycerol-6-phosphate transporter), | 1.492442849 | 1.59860 |
| member 4 \|\| Hs.132760 \|\| AA490159 \|\| \|\| 2542□ | | |
| 112948 \|\| CACNA1D \|\| calcium channel, voltage-dependent, L type, alpha 1D subunit \|\| | 1.479802344 | 3.64331 |
| Hs.399966 \|\| H29256 \|\| \|\| 776□ | | |
| 110391 \|\| WNT2 \|\| wingless-type MMTV integration site family member 2 \|\| Hs.89791 \|\| | 1.470522042 | 2.41407 |
| T99055 \|\| \|\| 7472□ | | |
| 107959 \|\| CCL3L2 \|\| **chemokine (C-C motif) ligand 3-like 2 \|\| Hs.512683 \|\| R47892 \|\| | 1.467347257 | 1.62489 |
| LD78 beta = almost identical to MIP-1 alpha = chemokine \|\| 390788□ | | |
| 224792 \|\| DPP8 \|\| dipeptidylpeptidase 8 \|\| Hs.439202 \|\| AA496257 \|\| \|\| 54878□ | 1.443587752 | 2.88507 |
| 116920 \|\| SMPD3 \|\| **sphingomyelin phosphodiesterase 3, neutral membrane (neutral | 1.419929837 | 2.55824 |
| sphingomyelinase \|\|) \|\| Hs.91753 \|\| AA460963 \|\| \|\| 55512□ | | |
| 106589 \|\| SLITRK3 \|\| SLIT and NTRK-like family, member 3 \|\| Hs.101745 \|\| H19422 \|\| \|\| | 1.404350541 | 3.01751 |
| 22865□ | | |
| 313462 \|\| FOXF1 \|\| forkhead box F1 \|\| Hs.155591 \|\| AA907419 \|\| \|\| 2294□ | 1.373996415 | 2.94356 |
| 117647 \|\| CG018 \|\| hypothetical gene CG018 \|\| Hs.277888 \|\| AA487590 \|\| \|\| 90634□ | 1.366231187 | 1.55600 |
| 220905 \|\| MSTP9 \|\| macrophage stimulating, pseudogene 9 \|\| Hs.475654 \|\| AA707273 \|\| | 1.358029773 | 1.69881 |
| \|\| 11223□ | | |
| 99229 \|\| EPS8 \|\| epidermal growth factor receptor pathway substrate 8 \|\| Hs.2132 \|\| | 1.352259468 | 1.58170 |
| H13622 \|\| epidermal growth factor receptor kinase substrate (Eps8) \|\| 2059□ | | |
| 310096 \|\| FLJ12604 \|\| hypothetical protein FLJ12604 \|\| Hs.126485 \|\| AI015671 \|\| \|\| | 1.345723012 | 2.91593 |
| 79674□ | | |
| 224759 \|\| MYBL1 \|\| v-myb myeloblastosis viral oncogene homolog (avian)-like 1 \|\| | 1.334358929 | 2.06935 |
| Hs.300592 \|\| AA911236 \|\| \|\| 4603□ | | |
| 108082 \|\| SYNPO \|\| synaptopodin \|\| Hs.511770 \|\| H49442 \|\| \|\| 11346□ | 1.333170303 | 3.58591 |
| 110153 \|\| ODZ4 \|\| odz, odd Oz/ten-m homolog 4 (Drosophila) \|\| Hs.5028 \|\| AA449657 \|\| | 1.299117442 | 3.23864 |
| \|\| 26011□ | | |
| 116829 \|\| \|\| CDNA clone MGC: 52263 IMAGE: 4123447, complete cds \|\| Hs.251664 \|\| | 1.298123994 | 3.86505 |
| N76677 \|\| \|\| □ | | |
| 100381 \|\| CDS1 \|\| CDP-diacylglycerol synthase (phosphatidate cytidylyltransferase) 1 \|\| | 1.298003078 | 1.44849 |
| Hs.380684 \|\| R31300 \|\| \|\| 1040□ | | |
| 102111 \|\| GALNT13 \|\| UDP-N-acetyl-alpha-D-galactosamine: polypeptide N- | 1.295025758 | 3.29729 |
| acetylgalactosaminyltransferase 13 (GalNAc-T13) \|\| Hs.13485 \|\| H23028 \|\| \|\| 114805□ | | |
| 226498 \|\| \|\| CDNA FLJ27142 fis, clone SPL08955 \|\| Hs.38207 \|\| AA777567 \|\| \|\| | 1.29045908 | 2.44492 |
| 200230□ | | |
| 114904 \|\| CCL2 \|\| chemokine (C-C motif) ligand 2 \|\| Hs.303649 \|\| AA425102 \|\| MCP- | 1.286358059 | 2.08760 |
| 1 = MCAF = small inducible cytokine A2 = JE = chemokine \|\| 6347□ | | |
| 108777 \|\| WNT2 \|\| wingless-type MMTV integration site family member 2 \|\| Hs.89791 \|\| | 1.282412015 | 2.23550 |
| H04382 \|\| WNT-2 \|\| 7472□ | | |
| 112346 \|\| PHYHIP \|\| phytanoyl-CoA hydroxylase interacting protein \|\| Hs.334688 \|\| | 1.274201347 | 2.03025 |
| AA405628 \|\| \|\| 9796□ | | |
| 308017 \|\| B3GTL \|\| beta 3-glycosyltransferase-like \|\| Hs.13205 \|\| AA937150 \|\| \|\| | 1.269281165 | 4.77100 |
| 145173□ | | |
| 117427 \|\| SOX9 \|\| SRY (sex determining region Y)-box 9 (campomelic dysplasia, | 1.26609229 | 1.76872 |
| autosomal sex-reversal) \|\| Hs.2316 \|\| AA400464 \|\| \|\| 6662□ | | |
| 226208 \|\| \|\| CDNA FLJ12777 fis, clone NT2RP2001720 \|\| Hs.445239 \|\| AA922858 \|\| \|\| □ | 1.264748051 | 2.94000 |
| 222412 \|\| ITGA4 \|\| **integrin, alpha 4 (antigen CD49D, alpha 4 subunit of VLA-4 | 1.264557885 | 2.29366 |
| receptor) \|\| Hs.528404 \|\| AA490846 \|\| \|\| 3676□ | | |
| 317940 \|\| ITGA4 \|\| **integrin, alpha 4 (antigen CD49D, alpha 4 subunit of VLA-4 | 1.264070332 | 2.74126 |
| receptor) \|\| Hs.528404 \|\| W73004 \|\| \|\| 3676□ | | |
| 112364 \|\| \|\| Transcribed sequences \|\| Hs.436897 \|\| AA916872 \|\| \|\| □ | 1.259881012 | 2.57268 |
| 222035 \|\| \|\| \|\| \|\| AA259115 \|\| \|\| □ | 1.247144881 | 1.69678 |
| 112155 \|\| MAPK10 \|\| mitogen-activated protein kinase 10 \|\| Hs.25209 \|\| R39221 \|\| | 1.244006532 | 2.41273 |
| JNK3 = Stress-activated protein kinase \|\| 5602□ | | |
| 220478 \|\| \|\| MRNA; cDNA DKFZp564B182 (from clone DKFZp564B182) \|\| Hs.50150 \|\| | 1.235527813 | 2.29219 |
| W92438 \|\| \|\| □ | | |
| 318965 \|\| \|\| Transcribed sequences \|\| Hs.506212 \|\| AI335854 \|\| \|\| □ | 1.216895802 | 1.34269 |
| 110839 \|\| \|\| \|\| \|\| AA670429 \|\| \|\| □ | 1.211850654 | 1.55101 |
| 222279 \|\| B3GTL \|\| beta 3-glycosyltransferase-like \|\| Hs.13205 \|\| N24070 \|\| \|\| 145173□ | 1.201785812 | 2.82387 |
| 104022 \|\| PCSK1 \|\| **proprotein convertase subtilisin/kexin type 1 \|\| Hs.78977 \|\| R42630 | 1.180682992 | 2.57997 |
| \|\| \|\| 5122□ | | |
| 104652 \|\| VCAM1 \|\| vascular cell adhesion molecule 1 \|\| Hs.109225 \|\| H16591 \|\| | 1.174970539 | 3.09607 |
| CD106 = VCAM-1 \|\| 7412□ | | |
| 113996 \|\| COL9A3 \|\| collagen, type IX, alpha 3 \|\| Hs.126248 \|\| AA873567 \|\| \|\| 1299□ | 1.164364159 | 2.67626 |
| 102537 \|\| \|\| \|\| \|\| R69584 \|\| \|\| □ | 1.157582924 | 1.96363 |
| 104879 \|\| BBS2 \|\| Bardet-Biedl syndrome 2 \|\| Hs.333738 \|\| N93740 \|\| \|\| 583□ | 1.157393817 | 2.24818 |
| 99434 \|\| FMN2 \|\| formin 2 \|\| Hs.24889 \|\| R73539 \|\| \|\| 56776□ | 1.154515482 | 1.14395 |
| 224483 \|\| \|\| Transcribed sequences \|\| Hs.454845 \|\| AA777098 \|\| \|\| □ | 1.136611133 | 1.70069 |
| 112190 \|\| \|\| \|\| \|\| R75884 \|\| \|\| □ | 1.135753513 | 1.77431 |
| 118455 \|\| ABHD2 \|\| abhydrolase domain containing 2 \|\| Hs.412022 \|\| AA454207 \|\| \|\| | 1.118914849 | 7.06166 |
| 11057□ | | |
| 221321 \|\| \|\| \|\| \|\| AA876411 \|\| \|\| □ | 1.114362912 | 2.37843 |
| 118239 \|\| \|\| CDNA FLJ42140 fis, clone TESTI2041734 \|\| Hs.55982 \|\| AA284279 \|\| \|\| □ | 1.109133617 | 2.27783 |
| 223371 \|\| TRA2A \|\| transformer-2 alpha \|\| Hs.445652 \|\| AA465172 \|\| \|\| 29896□ | 1.096518274 | 1.54208 |
| 119406 \|\| ZNF533 \|\| zinc finger protein 533 \|\| Hs.6295 \|\| AA460346 \|\| \|\| 151126□ | 1.095919809 | 1.70692 |
| 247798 \|\| \|\| \|\| \|\| W01204 \|\| ADRENAL SPECIFIC 30 KD PROTEIN \|\| □ | 1.093316012 | 4.27986 |
| 117061 \|\| CD36 \|\| CD36 antigen (collagen type I receptor, thrombospondin receptor) \|\| | 1.049799708 | 1.78335 |
| Hs.443120 \|\| N39161 \|\| CD36 \|\| 948□ | | |
| 104146 \|\| ITPR2 \|\| inositol 1,4,5-triphosphate receptor, type 2 \|\| Hs.512235 \|\| AA479093 | 1.034722098 | 1.23677 |
| \|\| \|\| 3709□ | | |
| 106378 \|\| PRO1073 \|\| PRO1073 protein \|\| Hs.187199 \|\| AA064973 \|\| \|\| 29005□ | 1.032675937 | 2.58580 |
| 313485 \|\| \|\| \|\| \|\| AI287555 \|\| \|\| □ | 1.030127985 | 1.59590 |
| 98863 \|\| EFEMP1 \|\| EGF-containing fibulin-like extracellular matrix protein 1 \|\| Hs.76224 | 1.029667793 | 2.02334 |
| \|\| AA875933 \|\| \|\| 2202□ | | |
| 222670 \|\| CSPG2 \|\| chondroitin sulfate proteoglycan 2 (versican) \|\| Hs.434488 \|\| | −9.592197875 | 0.02050 |
| AA857944 \|\| \|\| 1462□ | | |
| 104875 \|\| \|\| \|\| \|\| AA669383 \|\| \|\| □ | −8.517832652 | 0.03484 |
| 119597 \|\| S100A10 \|\| S100 calcium binding protein A10 (annexin \|\| ligand, calpactin I, | −8.35582717 | 0.03148 |
| light polypeptide (p11)) \|\| Hs.143873 \|\| AA444051 \|\| \|\| 6281□ | | |
| 112583 \|\| POSTN \|\| periostin, osteoblast specific factor \|\| Hs.136348 \|\| AA598653 \|\| | −7.990063735 | 0.01449 |
| OSF-2os = osteoblast-specific factor = putative bone adhesion pr \|\| 10631□ | | |
| 104759 \|\| S100A10 \|\| **S100 calcium binding protein A10 (annexin \|\| ligand, calpactin I, | −7.84951104 | 0.03807 |
| light polypeptide (p11)) \|\| Hs.143873 \|\| AA873230 \|\| \|\| 6281□ | | |
| 225715 \|\| \|\| \|\| \|\| AA699878 \|\| \|\| □ | −7.669895604 | 0.01345 |
| 100797 \|\| THBS2 \|\| thrombospondin 2 \|\| Hs.458354 \|\| H38013 \|\| \|\| 7058□ | −7.599032817 | 0.03742 |
| 103873 \|\| NBL1 \|\| neuroblastoma, suppression of tumorigenicity 1 \|\| Hs.439671 \|\| | −7.367185665 | 0.01908 |
| AA598830 \|\| \|\| 4681□ | | |
| 220604 \|\| CTHRC1 \|\| collagen triple helix repeat containing 1 \|\| Hs.283713 \|\| AA406425 | −7.35763878 | 0.00867 |
| \|\| \|\| 115908□ | | |
| 119133 \|\| \|\| \|\| \|\| AA461456 \|\| \|\| □ | −7.332591357 | 0.03955 |
| 112166 \|\| MFAP2 \|\| microfibrillar-associated protein 2 \|\| Hs.389137 \|\| N67487 \|\| \|\| | −7.021772241 | 0.03326 |
| 4237□ | | |
| 308692 \|\| POSTN \|\| periostin, osteoblast specific factor \|\| Hs.136348 \|\| AI262129 \|\| \|\| | −6.998984665 | 0.00689 |
| 10631□ | | |
| 309448 \|\| COL5A2 \|\| collagen, type V, alpha 2 \|\| Hs.283393 \|\| AA857098 \|\|\|\| 1290□ | −6.946332088 | 0.03904 |
| 120018 \|\| C5orf13 \|\| chromosome 5 open reading frame 13 \|\| Hs.508741 \|\| H80684 \|\| \|\| | −6.911722459 | 0.04096 |
| 9315□ | | |
| 117509 \|\| ADAM12 \|\| a disintegrin and metalloproteinase domain 12 (meltrin alpha) \|\| | −6.882803652 | 0.02822 |
| Hs.8850 \|\| AA099554 \|\| \|\| 8038□ | | |
| 102967 \|\| CSPG2 \|\| chondroitin sulfate proteoglycan 2 (versican) \|\| Hs.434488 \|\| | −6.856057041 | 0.03060 |
| AA098997 \|\| \|\| 1462□ | | |
| 222543 \|\| COL8A1 \|\| collagen, type VIII, alpha 1 \|\| Hs.114599 \|\| N51859 \|\| \|\| 1295□ | −6.847387516 | 0.02934 |
| 114989 \|\| ANTXR1 \|\| anthrax toxin receptor 1 \|\| Hs.274520 \|\| N20989 \|\| \|\| 84168□ | −6.76088624 | 0.07115 |
| 116414 \|\| CNTN1 \|\| contactin 1 \|\| Hs.143434 \|\| H19023 \|\| \|\| 1272□ | −6.750724274 | 0.02115 |
| 112222 \|\| FLJ14464 \|\| hypothetical protein FLJ14464 \|\| Hs.348609 \|\| AI002047 \|\| \|\| | −6.672036387 | 0.06309 |
| 84875□ | | |
| 115748 \|\| CNTN3 \|\| contactin 3 (plasmacytoma associated) \|\| Hs.512593 \|\| N50845 \|\| \|\| | −6.635018303 | 0.06190 |
| 5067□ | | |
| 111379 \|\| LRRC15 \|\| leucine rich repeat containing 15 \|\| Hs.288467 \|\| AA449577 \|\| \|\| | −6.599602185 | 0.01545 |
| 131578□ | | |
| 312399 \|\| SULF1 \|\| sulfatase 1 \|\| Hs.409602 \|\| AI653116 \|\| \|\| 23213□ | −6.549349052 | 0.02781 |
| 119443 \|\| MAFB \|\| v-maf musculoaponeurotic fibrosarcoma oncogene homolog B | −6.500333359 | 0.04401 |
| (avian) \|\| Hs.169487 \|\| T50121 \|\| \|\| 9935□ | | |
| 114016 \|\| GJA1 \|\| gap junction protein, alpha 1, 43 kDa (connexin 43) \|\| Hs.74471 \|\| | −6.456842836 | 0.03476 |
| AA487623 \|\| \|\| 2697□ | | |
| 222120 \|\| LRRN1 \|\| leucine rich repeat neuronal 1 \|\| Hs.126085 \|\| AA778089 \|\| \|\| | −6.438088308 | 0.03558 |
| 57633□ | | |
| 112861 \|\| COL6A2 \|\| collagen, type VI, alpha 2 \|\| Hs.420269 \|\| AA464042 \|\| \|\| 1292□ | −6.220599318 | 0.06074 |
| 310794 \|\| LOC374946 \|\| hypothetical gene supported by AK075558; BC021286 \|\| | −6.22035749 | 0.01531 |
| Hs.371716 \|\| AA974305 \|\| \|\| 374946□ | | |
| 113487 \|\| CXCL14 \|\| chemokine (C-X-C motif) ligand 14 \|\| Hs.24395 \|\| W72294 \|\| \|\| | −6.210193072 | 0.03071 |
| 9547□ | | |
| 222179 \|\| SPON1 \|\| spondin 1, extracellular matrix protein \|\| Hs.5378 \|\| AA427924 \|\| \|\| | −6.168351147 | 0.04879 |
| 10418□ | | |
| 114548 \|\| BASP1 \|\| brain abundant, membrane attached signal protein 1 \|\| Hs.511745 \|\| | −6.14964011 | 0.04629 |
| AA488676 \|\| \|\| 10409□ | | |
| 114153 \|\| \|\| MRNA; cDNA DKFZp686P14145 (from clone DKFZp686P14145) \|\| | −6.092017164 | 0.06593 |
| Hs.42572 \|\| AA046112 \|\| \|\| □ | | |
| 103510 \|\| SPARC \|\| secreted protein, acidic, cysteine-rich (osteonectin) \|\| Hs.111779 \|\| | −6.079951397 | 0.03952 |
| AA046533 \|\| osteonectin = SPARC = basement membrane protein \|\| 6678□ | | |
| 101494 \|\| \|\| \|\| \|\| N71920 \|\| \|\| □ | −6.040966262 | 0.05049 |
| 112588 \|\| FARP1 \|\| FERM, RhoGEF (ARHGEF) and pleckstrin domain protein 1 | −6.034567012 | 0.07532 |
| (chondrocyte-derived) \|\| Hs.207428 \|\| AA496796 \|\| \|\| 10160□ | | |
| 108807 \|\| ITGB5 \|\| integrin, beta 5 \|\| Hs.149846 \|\| AA434397 \|\| \|\| 3693□ | −6.003462903 | 0.06776 |
| 226071 \|\| TM4SF13 \|\| transmembrane 4 superfamily member 13 \|\| Hs.364544 \|\| | −5.968408151 | 0.04523 |
| W86201 \|\| \|\| 27075□ | | |
| 110318 \|\| GREM1 \|\| gremlin 1 homolog, cysteine knot superfamily (Xenopus laevis) \|\| | −5.864270316 | 0.03347 |
| Hs.40098 \|\| W48619 \|\| \|\| 26585□ | | |
| 117138 \|\| COL3A1 \|\| collagen, type III, alpha 1 (Ehlers-Danlos syndrome type IV, | −5.851759851 | 0.05781 |
| autosomal dominant) \|\| Hs.443625 \|\| W90740 \|\| \|\| 1281□ | | |
| 221607 \|\| MDK \|\| midkine (neurite growth-promoting factor 2) \|\| Hs.82045 \|\| AA968896 \|\| | −5.788936868 | 0.05640 |
| \|\| 4192□ | | |
| 110328 \|\| PODN \|\| podocan \|\| Hs.136664 \|\| AA447610 \|\| \|\| 127435□ | −5.786438596 | 0.05070 |
| 220208 \|\| GREM1 \|\| gremlin 1 homolog, cysteine knot superfamily (Xenopus laevis) \|\| | −5.778888352 | 0.08664 |
| Hs.40098 \|\| W51909 \|\| \|\| 26585□ | | |
| 226840 \|\| \|\| \|\| \|\| T99175 \|\| \|\| □ | −5.762163039 | 0.06614 |
| 110351 \|\| \|\| CDNA FLJ39997 fis, clone STOMA2002367 \|\| Hs.127146 \|\| AA937728 \|\| \|\| □ | −5.756171175 | 0.05813 |
| 309986 \|\| \|\| \|\| \|\| AI057267 \|\| \|\| □ | −5.727209799 | 0.05015 |
| 98983 \|\| KIAA0574 \|\| KIAA0574 protein \|\| Hs.383564 \|\| R60151 \|\| \|\| 23359□ | −5.659466193 | 0.05894 |
| 108628 \|\| ADAM12 \|\| a disintegrin and metalloproteinase domain 12 (meltrin alpha) \|\| | −5.628607156 | 0.03143 |
| Hs.8850 \|\| AA190508 \|\| \|\| 8038□ | | |
| 106203 \|\| \|\| **Transcribed sequence with weak similarity to protein pir: S57447 | −5.59546726 | 0.04259 |
| (H. sapiens) S57447 HPBRII-7 protein - human \|\| Hs.47026 \|\| AA487845 \|\| \|\| □ | | |
| 99425 \|\| \|\| Transcribed sequences \|\| Hs.529878 \|\| AA922939 \|\| \|\| □ | −5.581513187 | 0.07144 |
| 318541 \|\| WISP1 \|\| WNT1 inducible signaling pathway protein 1 \|\| Hs.194680 \|\| | −5.568488377 | 0.05404 |
| AA922800 \|\| \|\| 8840□ | | |
| 102573 \|\| COL1A1 \|\| collagen, type I, alpha 1 \|\| Hs.172928 \|\| W90359 \|\| \|\| 1277□ | −5.564397065 | 0.05041 |
| 114266 \|\| PRDX4 \|\| peroxiredoxin 4 \|\| Hs.83383 \|\| AA459663 \|\| \|\| 10549□ | −5.538485188 | 0.09449 |
| 103176 \|\| COL3A1 \|\| collagen, type III, alpha 1 (Ehlers-Danlos syndrome type IV, | −5.533694161 | 0.04893 |
| autosomal dominant) \|\| Hs.443625 \|\| AA044829 \|\| \|\| 1281□ | | |
| 106329 \|\| CSRP2 \|\| cysteine and glycine-rich protein 2 \|\| Hs.10526 \|\| T59334 \|\| \|\| 1466□ | −5.525434515 | 0.06738 |
| 117004 \|\| FLJ12442 \|\| hypothetical protein FLJ12442 \|\| Hs.84753 \|\| R42815 \|\| \|\| 64943□ | −5.516000677 | 0.08060 |
| 314338 \|\| \|\| Transcribed sequence with weak similarity to protein sp: P39194 | −5.477445468 | 0.03432 |
| (H. sapiens) ALU7_HUMAN Alu subfamily SQ sequence contamination warning entry \|\| | | |
| Hs.270149 \|\| AA953560 \|\| \|\| □ | | |
| 105588 \|\| SERPINH1 \|\| serine (or cysteine) proteinase inhibitor, clade H (heat shock | −5.462286624 | 0.08542 |
| protein 47), member 1, (collagen binding protein 1) \|\| Hs.241579 \|\| R71093 \|\| \|\| 871□ | | |
| 105129 \|\| TMEPAI \|\| transmembrane, prostate androgen induced RNA \|\| Hs.83883 \|\| | −5.456310418 | 0.07672 |
| AA088701 \|\| \|\| 56937□ | | |
| 312141 \|\| ARL7 \|\| ADP-ribosylation factor-like 7 \|\| Hs.111554 \|\| AA281534 \|\| \|\| 10123□ | −5.440003276 | 0.07728 |
| 225651 \|\| EMID1 \|\| EMI domain containing 1 \|\| Hs.289106 \|\| AA775576 \|\| \|\| 129080□ | −5.436554585 | 0.06609 |
| 102116 \|\| THY1 \|\| Thy-1 cell surface antigen \|\| Hs.134643 \|\| AI346653 \|\| \|\| 7070□ | −5.432243879 | 0.05120 |
| 105119 \|\| COL1A2 \|\| collagen, type I, alpha 2 \|\| Hs.232115 \|\| AA490172 \|\| \|\| 1278□ | −5.406517109 | 0.06378 |
| 113048 \|\| PRC1 \|\| **protein regulator of cytokinesis 1 \|\| Hs.344037 \|\| AA449593 \|\| \|\| | −5.364911061 | 0.08599 |
| 9055□ | | |
| 109254 \|\| MTR \|\| 5-methyltetrahydrofolate-homocysteine methyltransferase \|\| Hs.82283 | −5.352851544 | 0.07307 |
| \|\| AA233640 \|\| \|\| 4548□ | | |
| 103712 \|\| GREM1 \|\| gremlin 1 homolog, cysteine knot superfamily (Xenopus laevis) \|\| | −5.351333158 | 0.04584 |
| Hs.40098 \|\| W47324 \|\| \|\| 26585□ | | |
| 116682 \|\| ECM1 \|\| extracellular matrix protein 1 \|\| Hs.81071 \|\| N79484 \|\| \|\| 1893□ | −5.300548395 | 0.07250 |
| 310273 \|\| SDC1 \|\| syndecan 1 \|\| Hs.82109 \|\| AI015641 \|\| \|\| 6382□ | −5.292278827 | 0.06135 |
| 224694 \|\| FHOD3 \|\| formin homology 2 domain containing 3 \|\| Hs.444746 \|\| H22559 \|\| \|\| | −5.274725515 | 0.05714 |
| 80206□ | | |
| 116205 \|\| TMEPAI \|\| transmembrane, prostate androgen induced RNA \|\| Hs.83883 \|\| | −5.252957638 | 0.08051 |
| AA455519 \|\| \|\| 56937□ | | |
| 111515 \|\| SPARC \|\| **secreted protein, acidic, cysteine-rich (osteonectin) \|\| Hs.111779 \|\| | −5.237004679 | 0.07100 |
| AA031596 \|\| \|\| 6678□ | | |
| 101660 \|\| FOSL2 \|\| FOS-like antigen 2 \|\| Hs.301612 \|\| N34799 \|\| fra-2 = fos-related | −5.226225018 | 0.08006 |
| antigen 2 \|\| 2355□ | | |
| 103827 \|\| LOXL1 \|\| lysyl oxidase-like 1 \|\| Hs.65436 \|\| AA405804 \|\| \|\| 4016□ | −5.219192079 | 0.06612 |
| 113575 \|\| DKFZP434B044 \|\| hypothetical protein DKFZp434B044 \|\| Hs.262958 \|\| | −5.207051512 | 0.08971 |
| AA460304 \|\| \|\| 83716□ | | |
| 111770 \|\| VMP1 \|\| likely ortholog of rat vacuole membrane protein 1 \|\| Hs.166254 \|\| | −5.191575643 | 0.08692 |
| AA485373 \|\| \|\| 81671□ | | |
| 100977 \|\| CDH11 \|\| cadherin 11, type 2, OB-cadherin (osteoblast) \|\| Hs.443435 \|\| | −5.185095143 | 0.07569 |
| AA137109 \|\| \|\| 1009□ | | |
| 105460 \|\| WDTC1 \|\| **WD and tetratricopeptide repeats 1 \|\| Hs.172825 \|\| AA004204 \|\| \|\| | −5.148395166 | 0.04953 |
| 23038□ | | |
| 111691 \|\| EDNRA \|\| endothelin receptor type A \|\| Hs.211202 \|\| AA450009 \|\| \|\| 1909□ | −5.14729003 | 0.07970 |
| 223266 \|\| SMOC2 \|\| SPARC related modular calcium binding 2 \|\| Hs.22209 \|\| AA931725 | −5.138874419 | 0.06508 |
| \|\| \|\| 64094□ | | |
| 103637 \|\| ADAM12 \|\| a disintegrin and metalloproteinase domain 12 (meltrin alpha) \|\| | −5.112867661 | 0.04883 |
| Hs.8850 \|\| H78537 \|\| \|\| 8038□ | | |
| 107937 \|\| CSPG2 \|\| chondroitin sulfate proteoglycan 2 (versican) \|\| Hs.434488 \|\| | −5.111074087 | 0.06139 |
| AA056022 \|\| \|\| 1462□ | | |
| 310261 \|\| \|\| Transcribed sequence with weak similarity to protein pir: T17227 | −5.108556844 | 0.07927 |
| (H. sapiens) T17227 hypothetical protein DKFZp434A236.1 - human \|\| Hs.131004 \|\| | | |
| AI015591 \|\| \|\| □ | | |
| 115949 \|\| SPON1 \|\| spondin 1, extracellular matrix protein \|\| Hs.5378 \|\| H09099 \|\| \|\| | −5.092981511 | 0.05822 |
| 10418□ | | |
| 111790 \|\| COL3A1 \|\| collagen, type III, alpha 1 (Ehlers-Danlos syndrome type IV, | −5.084132824 | 0.03798 |
| autosomal dominant) \|\| Hs.443625 \|\| AI679372 \|\| \|\| 1281□ | | |
| 106575 \|\| C5orf13 \|\| chromosome 5 open reading frame 13 \|\| Hs.508741 \|\| N91952 \|\| \|\| | −5.056963448 | 0.10420 |
| 9315□ | | |
| 225055 \|\| \|\| \|\| \|\| AA701863 \|\| \|\| □ | −5.011553933 | 0.10678 |
| 108598 \|\| \|\| \|\| \|\| AA908645 \|\| \|\| □ | −5.009183381 | 0.11236 |
| 113067 \|\| MGC16121 \|\| hypothetical protein MGC16121 \|\| Hs.416379 \|\| R32847 \|\| \|\| | −4.97944286 | 0.11709 |
| 84848□ | | |
| 107611 \|\| C14orf132 \|\| chromosome 14 open reading frame 132 \|\| Hs.458321 \|\| H21039 | −4.968449581 | 0.10101 |
| \|\| \|\| 56967□ | | |
| 103133 \|\| CTSC \|\| cathepsin C \|\| Hs.128065 \|\| AA740376 \|\| \|\| 1075□ | −4.953535018 | 0.10669 |
| 106624 \|\| RAB31 \|\| RAB31, member RAS oncogene family \|\| Hs.223025 \|\| T96082 \|\| \|\| | −4.928165715 | 0.11378 |
| 11031□ | | |
| 104911 \|\| \|\| Clone IMAGE: 5742072, mRNA \|\| Hs.133160 \|\| N22836 \|\| \|\| 401237□ | −4.919823563 | 0.08371 |
| 115298 \|\| KDELR3 \|\| KDEL (Lys-Asp-Glu-Leu) endoplasmic reticulum protein retention | −4.910903525 | 0.12770 |
| receptor 3 \|\| Hs.528305 \|\| AA181085 \|\| \|\| 11015□ | | |
| 226683 \|\| PDLIM3 \|\| PDZ and LIM domain 3 \|\| Hs.71719 \|\| AA972352 \|\| \|\| 27295□ | −4.907575601 | 0.07427 |
| 116645 \|\| THY1 \|\| Thy-1 cell surface antigen \|\| Hs.134643 \|\| AA877226 \|\| \|\| 7070□ | −4.893327096 | 0.05119 |
| 115718 \|\| FOSL2 \|\| FOS-like antigen 2 \|\| Hs.301612 \|\| N80371 \|\| \|\| 2355□ | −4.874358905 | 0.09148 |
| 310491 \|\| GREM1 \|\| gremlin 1 homolog, cysteine knot superfamily (Xenopus laevis) \|\| | −4.844566973 | 0.01713 |
| Hs.40098 \|\| AI041650 \|\| \|\| 26585□ | | |
| 108262 \|\| COL3A1 \|\| collagen, type III, alpha 1 (Ehlers-Danlos syndrome type IV, | −4.830982451 | 0.03607 |
| autosomal dominant) \|\| Hs.443625 \|\| T98611 \|\| \|\| 1281□ | | |
| 109540 \|\| WWP2 \|\| Nedd-4-like ubiquitin-protein ligase \|\| Hs.315485 \|\| H68403 \|\| \|\| | −4.829651358 | 0.11318 |
| 11060□ | | |
| 313617 \|\| CDH11 \|\| cadherin 11, type 2, OB-cadherin (osteoblast) \|\| Hs.443435 \|\| | −4.806952558 | 0.09042 |
| AI278518 \|\| \|\| 1009□ | | |
| 111509 \|\| PHLDB2 \|\| pleckstrin homology-like domain, family B, member 2 \|\| Hs.7378 \|\| | −4.804541226 | 0.10695 |
| AA479351 \|\| \|\| 90102□ | | |
| 226520 \|\| ADAM12 \|\| a disintegrin and metalloproteinase domain 12 (meltrin alpha) \|\| | −4.790180247 | 0.03894 |
| Hs.8850 \|\| AA702808 \|\| \|\| 8038□ | | |
| 226907 \|\| FREQ \|\| frequenin homolog (Drosophila) \|\| Hs.301760 \|\| AA918755 \|\| \|\| | −4.789446 | 0.12600 |
| 23413□ | | |
| 114280 \|\| KRT5 \|\| keratin 5 (epidermolysis bullosa simplex, Dowling- | −4.789168328 | 0.09799 |
| Meara/Kobner/Weber-Cockayne types) \|\| Hs.433845 \|\| W72110 \|\| \|\| 3852□ | | |
| 108290 \|\| NOPE \|\| likely ortholog of mouse neighbor of Punc E11 \|\| Hs.20924 \|\| N53427 | −4.772023487 | 0.08495 |
| \|\| \|\| 57722□ | | |
| 223960 \|\| HAK \|\| heart alpha-kinase \|\| Hs.388674 \|\| W47000 \|\| \|\| 115701□ | −4.749223522 | 0.08524 |
| 118377 \|\| TMEPAI \|\| transmembrane, prostate androgen induced RNA \|\| Hs.83883 \|\| | −4.738122574 | 0.09426 |
| AA486591 \|\| \|\| 56937□ | | |
| 105454 \|\| MARCKS \|\| myristoylated alanine-rich protein kinase C substrate \|\| Hs.318603 | −4.716510387 | 0.11579 |
| \|\| AA131320 \|\| \|\| 4082□ | | |
| 100405 \|\| B7H3 \|\| B7 homolog 3 \|\| Hs.77873 \|\| N54338 \|\| \|\| 80381□ | −4.708229968 | 0.12271 |
| 119752 \|\| OLFML2B \|\| olfactomedin-like 2B \|\| Hs.43658 \|\| N25353 \|\| \|\| 25903□ | −4.695623976 | 0.08508 |
| 118604 \|\| AMPH \|\| amphiphysin (Stiff-Man syndrome with breast cancer 128 kDa | −4.690979795 | 0.09676 |
| autoantigen) \|\| Hs.173034 \|\| H06483 \|\| \|\| 273□ | | |
| 99484 \|\| C1QTNF6 \|\| C1q and tumor necrosis factor related protein 6 \|\| Hs.22011 \|\| | −4.683996483 | 0.07127 |
| R16515 \|\| \|\| 114904□ | | |
| 224197 \|\| PMP22 \|\| peripheral myelin protein 22 \|\| Hs.372031 \|\| H26086 \|\| \|\| 5376□ | −4.671243437 | 0.13120 |
| 114908 \|\| KREMEN1 \|\| kringle containing transmembrane protein 1 \|\| Hs.229335 \|\| | −4.668133711 | 0.11863 |
| AA463206 \|\| \|\| 83999□ | | |
| 110390 \|\| FAM38B \|\| family with sequence similarity 38, member B \|\| Hs.293907 \|\| | −4.651259895 | 0.09725 |
| AA399973 \|\| \|\| 63895□ | | |
| 119714 \|\| COL5A2 \|\| collagen, type V, alpha 2 \|\| Hs.283393 \|\| AA599273 \|\| \|\| 1290□ | −4.628617827 | 0.11091 |
| 119785 \|\| SDC1 \|\| syndecan 1 \|\| Hs.82109 \|\| AA074511 \|\| \|\| 6382□ | −4.61400734 | 0.08125 |
| 112249 \|\| LRRC17 \|\| leucine rich repeat containing 17 \|\| Hs.288720 \|\| AA423870 \|\| \|\| | −4.575981297 | 0.10798 |
| 10234□ | | |
| 221880 \|\| DKFZp779O175 \|\| hypothetical protein DKFZp779O175 \|\| Hs.124047 \|\| | −4.575576934 | 0.13301 |
| AA625567 \|\| \|\| 374899□ | | |
| 119955 \|\| DKFZp761L1417 \|\| hypothetical protein DKFZp761L1417 \|\| Hs.270753 \|\| | −4.562981637 | 0.11167 |
| AA010589 \|\| \|\| 222865□ | | |
| 106872 \|\| DKFZp761B107 \|\| hypothetical protein DKFZp761B107 \|\| Hs.106432 \|\| | −4.5526047 | 0.13682 |
| R52679 \|\| \|\| 91050□ | | |
| 102767 \|\| \|\| \|\| \|\| AA829784 \|\| \|\| □ | −4.552315554 | 0.10927 |
| 101543 \|\| ADAM12 \|\| a disintegrin and metalloproteinase domain 12 (meltrin alpha) \|\| | −4.52804351 | 0.06277 |
| Hs.8850 \|\| AA035018 \|\| \|\| 8038□ | | |
| 105279 \|\| DSCR1L1 \|\| Down syndrome critical region gene 1-like 1 \|\| Hs.156007 \|\| | −4.521453744 | 0.10711 |
| H19439 \|\| \|\| 10231□ | | |
| 105938 \|\| ARL7 \|\| ADP-ribosylation factor-like 7 \|\| Hs.111554 \|\| N35301 \|\| \|\| 10123□ | −4.505167465 | 0.11391 |
| 111639 \|\| \|\| \|\| \|\| AA460239 \|\| \|\| □ | −4.501864871 | 0.09824 |
| 99271 \|\| ZNF521 zinc finger protein 521 \|\| Hs.280305 \|\| AA460732 \|\| \|\| 25925□ | −4.488567525 | 0.11092 |
| 100566 \|\| ST6GaIII \|\| beta-galactoside alpha-2,6-sialyltransferase \|\| \|\| Hs.98265 \|\| | −4.482693281 | 0.07320 |
| AA609458 \|\| \|\| 84620□ | | |
| 99836 \|\| FZD1 \|\| frizzled homolog 1 (Drosophila) \|\| Hs.94234 \|\| N70776 \|\| \|\| 8321□ | −4.476704208 | 0.10936 |
| 104992 \|\| FN1 \|\| fibronectin 1 \|\| Hs.418138 \|\| R62612 \|\| Fibronectin 1 \|\| 2335□ | −4.461777239 | 0.04475 |
| 114397 \|\| QPCT \|\| glutaminyl-peptide cyclotransferase (glutaminyl cyclase) \|\| Hs.79033 | −4.439483397 | 0.09370 |
| \|\| AA282134 \|\| \|\| 25797□ | | |
| 106499 \|\| \|\| Transcribed sequence with strong similarity to protein ref: NP_005442.2 | −4.436990729 | 0.14604 |
| (H. sapiens) enigma protein; LIM domain protein [Homo sapiens] \|\| Hs.509192 \|\| | | |
| AA125911 \|\| \|\| □ | | |
| 105845 \|\| COL1A2 \|\| collagen, type I, alpha 2 \|\| Hs.232115 \|\| W93067 \|\| \|\| 1278□ | −4.421867493 | 0.11384 |
| 116506 \|\| ARK5 \|\| AMP-activated protein kinase family member 5 \|\| Hs.200598 \|\| | −4.409607235 | 0.09879 |
| N92167 \|\| \|\| 9891□ | | |
| 102100 \|\| \|\| Transcribed sequences \|\| Hs.399719 \|\| N90913 \|\| \|\| □ | −4.405542003 | 0.13904 |
| 106281 \|\| EIF2B3 \|\| *eukaryotic translation initiation factor 2B, subunit 3 gamma, 58 kDa | −4.404344576 | 0.10030 |
| \|\| Hs.283627 \|\| W58367 \|\| \|\| 8891□ | | |
| 306860 \|\| ENTPD3 \|\| ectonucleoside triphosphate diphosphohydrolase 3 \|\| Hs.47042 \|\| | −4.396106445 | 0.10820 |
| AI290905 \|\| \|\| 956□ | | |
| 106504 \|\| \|\| \|\| \|\| R33355 \|\| \|\| □ | −4.393129073 | 0.11288 |
| 307956 \|\| LEPRE1 \|\| leucine proline-enriched proteoglycan (leprecan) 1 \|\| Hs.437656 \|\| | −4.38666028 | 0.15844 |
| AA954829 \|\| \|\| 64175□ | | |
| 109526 \|\| MGC16121 \|\| hypothetical protein MGC16121 \|\| Hs.416379 \|\| AA460825 \|\| \|\| | −4.382598112 | 0.13607 |
| 84848□ | | |
| 222583 \|\| SYTL2 \|\| synaptotagmin-like 2 \|\| Hs.390463 \|\| AA521439 \|\| \|\| 54843□ | −4.378399033 | 0.13558 |
| 111557 \|\| PDE4DIP \|\| phosphodiesterase 4D interacting protein (myomegalin) \|\| | −4.376803074 | 0.12310 |
| Hs.502577 \|\| AA192757 \|\| \|\| 9659□ | | |
| 112690 \|\| \|\| Transcribed sequence with moderate similarity to protein sp: Q01995 | −4.36185307 | 0.09527 |
| (H. sapiens) TAGL_HUMAN Transgelin \|\| Hs.512705 \|\| AA010664 \|\| \|\| □ | | |
| 103839 \|\| ANTXR1 \|\| anthrax toxin receptor 1 \|\| Hs.274520 \|\| H58644 \|\| \|\| 84168□ | −4.360763237 | 0.14867 |
| 104800 \|\| PCSK5 \|\| proprotein convertase subtilisin/kexin type 5 \|\| Hs.288931 \|\| | −4.354619202 | 0.11917 |
| AA256399 \|\| \|\| 5125□ | | |
| 100024 \|\| SFRP4 \|\| secreted frizzled-related protein 4 \|\| Hs.105700 \|\| AA486838 \|\| \|\| | −4.323669912 | 0.03445 |
| 6424□ | | |
| 317991 \|\| TNFRSF12A \|\| tumor necrosis factor receptor superfamily, member 12A \|\| | −4.321266255 | 0.11679 |
| Hs.355899 \|\| AI221536 \|\| \|\| 51330□ | | |
| 109983 \|\| FN1 \|\| fibronectin 1 \|\| Hs.418138 \|\| AI262682 \|\| \|\| 2335□ | −4.310113525 | 0.04959 |
| 107064 \|\| \|\| \|\| \|\| AA709414 \|\| \|\| □ | −4.308581605 | 0.13947 |
| 311177 \|\| LOX \|\| lysyl oxidase \|\| Hs.102267 \|\| H80736 \|\| \|\| 4015□ | −4.262927906 | 0.11131 |
| 106200 \|\| COL6A1 \|\| **collagen, type VI, alpha 1 \|\| Hs.415997 \|\| AA026618 \|\| \|\| 1291□ | −4.253849341 | 0.11047 |
| 108898 \|\| C14orf166 \|\| **chromosome 14 open reading frame 166 \|\| Hs.369840 \|\| | −4.249148971 | 0.15029 |
| AA478659 \|\| \|\| 51637□ | | |
| 99937 \|\| MCAM \|\| **melanoma cell adhesion molecule \|\| Hs.511397 \|\| AA489587 \|\| \|\| | −4.241357133 | 0.09123 |
| 4162□ | | |
| 120182 \|\| FBLN2 \|\| fibulin 2 \|\| Hs.198862 \|\| AA452840 \|\| \|\| 2199□ | −4.228492193 | 0.09121 |
| 108960 \|\| \|\| \|\| \|\| AA918703 \|\| \|\| □ | −4.223545758 | 0.14392 |
| 113533 \|\| COLEC12 \|\| collectin sub-family member 12 \|\| Hs.29423 \|\| N53421 \|\| \|\| | −4.220159353 | 0.07729 |
| 81035□ | | |
| 221623 \|\| LOC338773 \|\| hypothetical protein LOC338773 \|\| Hs.449718 \|\| AA702809 \|\| \|\| | −4.20785142 | 0.07588 |
| 338773□ | | |
| 225557 \|\| TNFRSF19 \|\| tumor necrosis factor receptor superfamily, member 19 \|\| | −4.194541083 | 0.11641 |
| Hs.334174 \|\| AA777555 \|\| \|\| 55504□ | | |
| 98861 \|\| MMP11 \|\| matrix metalloproteinase 11 (stromelysin 3) \|\| Hs.143751 \|\| | −4.191191655 | 0.08766 |
| AA040568 \|\| \|\| 4320□ | | |
| 112591 \|\| \|\| \|\| \|\| AA489616 \|\| \|\| □ | −4.174825534 | 0.12911 |
| 309804 \|\| EGFL3 \|\| EGF-like-domain, multiple 3 \|\| Hs.56186 \|\| AI084613 \|\| \|\| 1953□ | −4.170657388 | 0.08442 |
| 119005 \|\| RAB31 \|\| RAB31, member RAS oncogene family \|\| Hs.223025 \|\| AA449590 \|\| | −4.158380457 | 0.12461 |
| \|\| 11031□ | | |
| 100234 \|\| FOSL2 \|\| FOS-like antigen 2 \|\| Hs.301612 \|\| AA101616 \|\| \|\| 2355□ | −4.155827266 | 0.11860 |
| 223884 \|\| PRRX1 \|\| paired related homeobox 1 \|\| Hs.443452 \|\| AA663309 \|\| \|\| 5396□ | −4.137524618 | 0.11633 |
| 224867 \|\| MGC16121 \|\| hypothetical protein MGC16121 \|\| Hs.416379 \|\| AA634409 \|\| \|\| | −4.13653598 | 0.09925 |
| 84848□ | | |
| 115502 \|\| SPARC \|\| secreted protein, acidic, cysteine-rich (osteonectin) \|\| Hs.111779 \|\| | −4.134575243 | 0.06257 |
| H95959 \|\| \|\| 6678□ | | |
| 317231 \|\| \|\| Transcribed sequence with weak similarity to protein sp: P39194 | −4.127377514 | 0.12127 |
| (H. sapiens) ALU7_HUMAN Alu subfamily SQ sequence contamination warning entry \|\| | | |
| Hs.149458 \|\| AI279296 \|\| \|\| □ | | |
| 102847 \|\| FBN1 \|\| fibrillin 1 (Marfan syndrome) \|\| Hs.750 \|\| AA056415 \|\| \|\| 2200□ | −4.118592947 | 0.13125 |
| 223310 \|\| \|\| \|\| \|\| R48843 \|\| \|\| □ | −4.113151339 | 0.12454 |
| 117408 \|\| GOLPH2 \|\| **golgi phosphoprotein 2 \|\| Hs.352662 \|\| AA454597 \|\| \|\| 51280□ | −4.11037283 | 0.15348 |
| 107960 \|\| C5orf13 \|\| chromosome 5 open reading frame 13 \|\| Hs.508741 \|\| H60254 \|\| \|\| | −4.104011245 | 0.12475 |
| 9315□ | | |
| 221444 \|\| C9orf19 \|\| chromosome 9 open reading frame 19 \|\| Hs.302766 \|\| AA634164 \|\| | −4.091476214 | 0.13739 |
| \|\| 152007□ | | |
| 115550 \|\| NPTX2 \|\| neuronal pentraxin \|\| \|\| Hs.3281 \|\| AA683041 \|\| \|\| 4885□ | −4.072873281 | 0.13795 |
| 102526 \|\| \|\| CDNA FLJ44429 fis, clone UTERU2015653 \|\| Hs.86538 \|\| AA478747 \|\| \|\| □ | −4.071520333 | 0.08258 |
| 226007 \|\| \|\| **Transcribed sequences \|\| Hs.171965 \|\| R81486 \|\| \|\| □ | −4.063576923 | 0.13358 |
| 310753 \|\| LRRC17 \|\| leucine rich repeat containing 17 \|\| Hs.288720 \|\| AI341604 \|\| \|\| | −4.056459266 | 0.12595 |
| 10234□ | | |
| 308255 \|\| PLXNC1 \|\| plexin C1 \|\| Hs.286229 \|\| H98855 \|\| \|\| 10154□ | −4.050858783 | 0.11551 |
| 226857 \|\| C5orf13 \|\| chromosome 5 open reading frame 13 \|\| Hs.508741 \|\| AA707174 \|\| | −4.045340063 | 0.15005 |
| \|\| 9315□ | | |
| 225846 \|\| \|\| Hypothetical gene supported by AK123554 (LOC400517), mRNA \|\| | −4.025834617 | 0.11225 |
| Hs.366751 \|\| AA677668 \|\| \|\| 400517□ | | |
| 107766 \|\| \|\| \|\| \|\| AA004664 \|\| \|\| □ | −4.025661914 | 0.11634 |
| 106445 \|\| MGC9850 \|\| hypothetical protein MGC9850 \|\| Hs.222061 \|\| W95586 \|\| | −4.022982886 | 0.12302 |
| Unknown UG Hs.106127 ESTs, Moderately similar to (define \|\| 219404□ | | |
| 110424 \|\| XRCC1 \|\| X-ray repair complementing defective repair in Chinese hamster | −4.009334785 | 0.13479 |
| cells 1 \|\| Hs.98493 \|\| AA425139 \|\| \|\| 7515□ | | |
| 112604 \|\| ID3 \|\| inhibitor of DNA binding 3, dominant negative helix-loop-helix protein \|\| | −4.007978195 | 0.15640 |
| Hs.76884 \|\| AA482119 \|\| \|\| 3399□ | | |
| 101310 \|\| AKAP12 \|\| A kinase (PRKA) anchor protein (gravin) 12 \|\| Hs.197081 \|\| | −4.000165341 | 0.15736 |
| AA478542 \|\| \|\| 9590□ | | |
| 119834 \|\| LPHN2 \|\| latrophilin 2 \|\| Hs.24212 \|\| W74533 \|\| \|\| 23266□ | −3.997101975 | 0.14308 |
| 118034 \|\| IL1R1 \|\| interleukin 1 receptor, type I \|\| Hs.82112 \|\| AA464525 \|\| IL-1 receptor | −3.995156533 | 0.14302 |
| type I \|\| 3554□ | | |
| 311776 \|\| TPS1 \|\| tryptase, alpha \|\| Hs.405479 \|\| AI675311 \|\| \|\| 7176□ | −3.994689979 | 0.10501 |
| 116154 \|\| GUCY1A3 \|\| guanylate cyclase 1, soluble, alpha 3 \|\| Hs.433488 \|\| H22135 \|\| \|\| | −3.989739082 | 0.13455 |
| 2982□ | | |
| 105080 \|\| DKK3 \|\| dickkopf homolog 3 (Xenopus laevis) \|\| Hs.130865 \|\| AA425947 \|\| \|\| | −3.984686415 | 0.09493 |
| 27122□ | | |
| 309044 \|\| TWIST1 \|\| twist homolog 1 (acrocephalosyndactyly 3; Saethre-Chotzen | −3.98297472 | 0.13427 |
| syndrome) (Drosophila) \|\| Hs.66744 \|\| AI220198 \|\| \|\| 7291□ | | |
| 113638 \|\| MARCKS \|\| myristoylated alanine-rich protein kinase C substrate \|\| Hs.318603 | −3.933043456 | 0.14956 |
| \|\| AA482231 \|\| \|\| 4082□ | | |
| 109321 \|\| FARP1 \|\| FERM, RhoGEF (ARHGEF) and pleckstrin domain protein 1 | −3.92668658 | 0.14998 |
| (chondrocyte-derived) \|\| Hs.207428 \|\| AA486435 \|\| \|\| 10160□ | | |
| 319342 \|\| ADAM12 \|\| a disintegrin and metalloproteinase domain 12 (meltrin alpha) \|\| | −3.920869992 | 0.09702 |
| Hs.8850 \|\| R33104 \|\| \|\| 8038□ | | |
| 119999 \|\| KIAA0711 \|\| KIAA0711 gene product \|\| Hs.5333 \|\| AA702544 \|\| \|\| 9920□ | −3.919982776 | 0.16445 |
| 120342 \|\| \|\| \|\| \|\| W93154 \|\| \|\| □ | −3.918864544 | 0.16895 |
| 114315 \|\| \|\| Transcribed sequence with strong similarity to protein ref: NP_006033.1 | −3.909630845 | 0.13081 |
| (H. sapiens) heparan sulfate D-glucosaminyl 3-O-sulfotransferase 3A1; heparin- | | |
| glucosamine 3-O-sulfotransferase [Homo sapiens] \|\| Hs.458493 \|\| N59438 \|\| \|\| □ | | |
| 220140 \|\| SYTL2 \|\| synaptotagmin-like 2 \|\| Hs.390463 \|\| AA046565 \|\| \|\| 54843□ | −3.908532707 | 0.13527 |
| 120562 \|\| GPNMB \|\| glycoprotein (transmembrane) nmb \|\| Hs.389964 \|\| AA425450 \|\| \|\| | −3.906241366 | 0.14602 |
| 10457□ | | |
| 223889 \|\| WFDC1 \|\| WAP four-disulfide core domain 1 \|\| Hs.36688 \|\| AA150491 \|\| \|\| | −3.904115449 | 0.14501 |
| 58189□ | | |
| 105805 \|\| DSG2 \|\| desmoglein 2 \|\| Hs.412597 \|\| W37448 \|\| \|\| 1829□ | −3.901621475 | 0.14957 |
| 319726 \|\| COL8A1 \|\| collagen, type VIII, alpha 1 \|\| Hs.114599 \|\| AA664472 \|\| \|\| 1295□ | −3.899801051 | 0.11734 |
| 223850 \|\| SH3MD4 \|\| SH3 multiple domains 4 \|\| Hs.13254 \|\| H17370 \|\| \|\| 344558□ | −3.899632989 | 0.11005 |
| 119242 \|\| DAF \|\| decay accelerating factor for complement (CD55, Cromer blood group | −3.891490218 | 0.15094 |
| system) \|\| Hs.408864 \|\| R09561 \|\| CD55 = Decay accelerating factor \|\| 1604□ | | |
| 116870 \|\| INHBA \|\| inhibin, beta A (activin A, activin AB alpha polypeptide) \|\| Hs.28792 \|\| | −3.878569798 | 0.11241 |
| R66924 \|\| \|\| 3624□ | | |
| 115054 \|\| KAL1 \|\| Kallmann syndrome 1 sequence \|\| Hs.380850 \|\| H17882 \|\| \|\| 3730□ | −3.862214973 | 0.13581 |
| 99834 \|\| \|\| \|\| \|\| H99816 \|\| \|\| □ | −3.861564936 | 0.16234 |
| 221330 \|\| PRDM6 \|\| PR domain containing 6 \|\| Hs.135118 \|\| N29774 \|\| \|\| 93166□ | −3.860443812 | 0.13849 |
| 107861 \|\| THY1 \|\| Thy-1 cell surface antigen \|\| Hs.134643 \|\| AA428836 \|\| \|\| 7070□ | −3.852276216 | 0.16278 |
| 115654 \|\| SCUBE2 \|\| signal peptide, CUB domain, EGF-like 2 \|\| Hs.435861 \|\| AA574391 | −3.837775215 | 0.13466 |
| \|\| \|\| 57758□ | | |
| 226780 \|\| MMP23B \|\| matrix metalloproteinase 23B \|\| Hs.211819 \|\| AA151428 \|\| \|\| | −3.833998434 | 0.08966 |
| 8510□ | | |
| 220983 \|\| COL11A1 \|\| collagen, type XI, alpha 1 \|\| Hs.439168 \|\| AA775384 \|\| \|\| 1301□ | −3.816096447 | 0.11932 |
| 114704 \|\| C21orf56 \|\| **chromosome 21 open reading frame 56 \|\| Hs.381214 \|\| | −3.81601434 | 0.16380 |
| AA431571 \|\| \|\| 84221□ | | |
| 100373 \|\| AEBP1 \|\| AE binding protein 1 \|\| Hs.439463 \|\| AA490462 \|\| \|\| 165□ | −3.807858212 | 0.13337 |
| 99321 \|\| \|\| CDNA FLJ42250 fis, clone TKIDN2007828 \|\| Hs.22247 \|\| T50020 \|\| \|\| □ | −3.801246909 | 0.14763 |
| 109177 \|\| NAV1 \|\| neuron navigator 1 \|\| Hs.6298 \|\| AA411668 \|\| \|\| 89796□ | −3.798250154 | 0.15642 |
| 310094 \|\| SRPX2 \|\| sushi-repeat-containing protein, X-linked 2 \|\| Hs.306339 \|\| AI362134 | −3.796232437 | 0.16020 |
| \|\| \|\| 27286□ | | |
| 318571 \|\| URB \|\| steroid sensitive gene 1 \|\| Hs.356289 \|\| AI082183 \|\| \|\| 151887□ | −3.779400424 | 0.14387 |
| 107265 \|\| COL12A1 \|\| collagen, type XII, alpha 1 \|\| Hs.101302 \|\| AA478481 \|\| \|\| 1303□ | −3.762343725 | 0.12734 |
| 116399 \|\| SHB \|\| SHB (Src homology 2 domain containing) adaptor protein B \|\| | −3.761192325 | 0.17244 |
| Hs.379206 \|\| AA427595 \|\| \|\| 6461□ | | |
| 109176 \|\| \|\| \|\| \|\| T98151 \|\| \|\| □ | −3.760827415 | 0.14772 |
| 114187 \|\| ADAMTS1 \|\| a disintegrin-like and metalloprotease (reprolysin type) with | −3.746383605 | 0.16459 |
| thrombospondin type 1 motif, 1 \|\| Hs.8230 \|\| R76276 \|\| \|\| 9510□ | | |
| 109676 \|\| MGC15476 \|\| thymus expressed gene 3-like \|\| Hs.134185 \|\| W72525 \|\| \|\| | −3.741869811 | 0.17023 |
| 147906□ | | |
| 105209 \|\| KLF7 \|\| **Kruppel-like factor 7 (ubiquitous) \|\| Hs.436708 \|\| N49209 \|\| \|\| 8609□ | −3.740462151 | 0.18713 |
| 120642 \|\| PLXDC1 \|\| plexin domain containing 1 \|\| Hs.125036 \|\| H11476 \|\| \|\| 57125□ | −3.736046808 | 0.09926 |
| 116211 \|\| ELN \|\| elastin (supravalvular aortic stenosis, Williams-Beuren syndrome) \|\| | −3.729423407 | 0.08608 |
| Hs.252418 \|\| AA459308 \|\| \|\| 2006□ | | |
| 101031 \|\| MN1 \|\| meningioma (disrupted in balanced translocation) 1 \|\| Hs.268515 \|\| | −3.712940642 | 0.11399 |
| R59212 \|\| \|\| 4330□ | | |
| 223403 \|\| PERP \|\| PERP, TP53 apoptosis effector \|\| Hs.149620 \|\| AA775509 \|\| \|\| | −3.711848103 | 0.18933 |
| 64065□ | | |
| 106087 \|\| \|\| Transcribed sequences \|\| Hs.23850 \|\| N34849 \|\| \|\| □ | −3.709673343 | 0.16719 |
| 116802 \|\| DOC1 \|\| downregulated in ovarian cancer 1 \|\| Hs.15432 \|\| W69790 \|\| \|\| | −3.70786066 | 0.16017 |
| 11259□ | | |
| 102761 \|\| EDG2 \|\| endothelial differentiation, lysophosphatidic acid G-protein-coupled | −3.707254552 | 0.17012 |
| receptor, 2 \|\| Hs.75794 \|\| AA193405 \|\| \|\| 1902□ | | |
| 108694 \|\| KIAA0802 \|\| KIAA0802 \|\| Hs.434101 \|\| W55875 \|\| \|\| 23255□ | −3.707061391 | 0.15268 |
| 112967 \|\| SLC39A4 \|\| **solute carrier family 39 (zinc transporter), member 4 \|\| | −3.699766676 | 0.16283 |
| Hs.411274 \|\| AI017237 \|\| \|\| 55630□ | | |
| 109984 \|\| LOX \|\| lysyl oxidase \|\| Hs.102267 \|\| AA037732 \|\| \|\| 4015□ | −3.696337253 | 0.13771 |
| 104244 \|\| \|\| \|\| \|\| AA417659 \|\| \|\| □ | −3.664061691 | 0.14953 |
| 220472 \|\| C1QTNF6 \|\| C1q and tumor necrosis factor related protein 6 \|\| Hs.22011 \|\| | −3.662063446 | 0.12781 |
| AA677315 \|\| \|\| 114904□ | | |
| 222391 \|\| FHL2 \|\| four and a half LIM domains 2 \|\| Hs.8302 \|\| AA995282 \|\| \|\| 2274□ | −3.654501493 | 0.18619 |
| 112199 \|\| MYO10 \|\| myosin X \|\| Hs.61638 \|\| AA187977 \|\| \|\| 4651□ | −3.646259826 | 0.16295 |
| 107127 \|\| CNN1 \|\| calponin 1, basic, smooth muscle \|\| Hs.21223 \|\| AA398400 \|\| \|\| | −3.632534405 | 0.17562 |
| 1264□ | | |
| 223248 \|\| \|\| Transcribed sequences \|\| Hs.120852 \|\| AA705436 \|\| \|\| □ | −3.615838547 | 0.18480 |
| 119506 \|\| COL6A1 \|\| collagen, type VI, alpha 1 \|\| Hs.415997 \|\| H99676 \|\| \|\| 1291□ | −3.610879456 | 0.17070 |
| 101183 \|\| \|\| Clone IMAGE: 4791553, mRNA \|\| Hs.22907 \|\| AA973634 \|\| \|\| □ | −3.60129161 | 0.18485 |
| 119243 \|\| KREMEN1 \|\| **kringle containing transmembrane protein 1 \|\| Hs.229335 \|\| | −3.590771517 | 0.17861 |
| AA234669 \|\| \|\| 83999□ | | |
| 102605 \|\| MYL9 \|\| myosin, light polypeptide 9, regulatory \|\| Hs.433814 \|\| AA877166 \|\| \|\| | −3.586855819 | 0.18132 |
| 10398□ | | |
| 224656 \|\| ANGPTL2 \|\| angiopoietin-like 2 \|\| Hs.8025 \|\| AA704833 \|\| \|\| 23452□ | −3.574035138 | 0.15308 |
| 117192 \|\| BDKRB2 \|\| bradykinin receptor B2 \|\| Hs.250882 \|\| AA194043 \|\| \|\| 624□ | −3.56791306 | 0.15721 |
| 224080 \|\| DPT \|\| dermatopontin \|\| Hs.80552 \|\| AA626248 \|\| \|\| 1805□ | −3.567873487 | 0.14484 |
| 311459 \|\| NKD2 \|\| naked cuticle homolog 2 (Drosophila) \|\| Hs.240951 \|\| AI263890 \|\| \|\| | −3.552359807 | 0.19784 |
| 85409□ | | |
| 104765 \|\| \|\| CDNA FLJ11639 fis, clone HEMBA1004327 \|\| Hs.187578 \|\| AA055052 \|\| \|\| □ | −3.545516473 | 0.16830 |
| 105859 \|\| PRELP \|\| proline arginine-rich end leucine-rich repeat protein \|\| Hs.76494 \|\| | −3.539655863 | 0.15001 |
| AA434067 \|\| \|\| 5549□ | | |
| 100311 \|\| SDCCAG33 \|\| serologically defined colon cancer antigen 33 \|\| Hs.284217 \|\| | −3.535700135 | 0.16394 |
| N72048 \|\| \|\| 10194□ | | |
| 226785 \|\| ARK5 \|\| AMP-activated protein kinase family member 5 \|\| Hs.200598 \|\| | −3.534899587 | 0.12502 |
| AA774839 \|\| \|\| 9891□ | | |
| 116573 \|\| APCDD1 \|\| adenomatosis polyposis coli down-regulated 1 \|\| Hs.374481 \|\| | −3.533111075 | 0.11682 |
| W72005 \|\| \|\| 147495□ | | |
| 114184 \|\| URB \|\| steroid sensitive gene 1 \|\| Hs.356289 \|\| AA416767 \|\| \|\| 151887□ | −3.524455456 | 0.12939 |
| 118907 \|\| \|\| \|\| \|\| N39262 \|\| \|\| □ | −3.515931006 | 0.12588 |
| 106972 \|\| \|\| Transcribed sequence with weak similarity to protein pir: JC1087 | −3.502714971 | 0.21360 |
| (H. sapiens) JC1087 RNA helicase, ATP-dependent - human \|\| Hs.221931 \|\| AA035704 | | |
| \|\| \|\| □ | | |
| 109699 \|\| TCEB3 \|\| transcription elongation factor B (SIII), polypeptide 3 (110 kDa, | −3.498772673 | 0.15488 |
| elongin A) \|\| Hs.15535 \|\| AA128607 \|\| \|\| 6924□ | | |
| 309887 \|\| ADAMTS14 \|\| a disintegrin-like and metalloprotease (reprolysin type) with | −3.475485436 | 0.15850 |
| thrombospondin type 1 motif, 14 \|\| Hs.352156 \|\| W60649 \|\| \|\| 140766□ | | |
| 107953 \|\| \|\| **Transcribed sequence with moderate similarity to protein sp: P39194 | −3.473715268 | 0.17875 |
| (H. sapiens) ALU7_HUMAN Alu subfamily SQ sequence contamination warning entry \|\| | | |
| Hs.473082 \|\| AA665536 \|\| \|\| □ | | |
| 108781 \|\| GARP \|\| glycoprotein A repetitions predominant \|\| Hs.151641 \|\| AA122287 \|\| \|\| | −3.467891281 | 0.19211 |
| 2615□ | | |
| 112474 \|\| RNF144 \|\| ring finger protein 144 \|\| Hs.78894 \|\| W91930 \|\| \|\| 9781□ | −3.461514601 | 0.20429 |
| 101336 \|\| URB \|\| steroid sensitive gene 1 \|\| Hs.356289 \|\| AA455496 \|\| \|\| 151887□ | −3.46063246 | 0.14418 |
| 102898 \|\| \|\| Transcribed sequences \|\| Hs.444753 \|\| AA460314 \|\| \|\| □ | −3.457732296 | 0.19988 |
| 310664 \|\| AHR \|\| aryl hydrocarbon receptor \|\| Hs.170087 \|\| AA181307 \|\| \|\| 196□ | −3.454904044 | 0.15614 |
| 104753 \|\| TGFB3 \|\| **transforming growth factor, beta 3 \|\| Hs.2025 \|\| AA040616 \|\| \|\| | −3.445038371 | 0.16639 |
| 7043□ | | |
| 118506 \|\| \|\| MRNA; cDNA DKFZp586J1717 (from clone DKFZp586J1717) \|\| Hs.56027 \|\| | −3.441109124 | 0.15701 |
| W48600 \|\| \|\| □ | | |
| 223892 \|\| PCYOX1 \|\| prenylcysteine oxidase 1 \|\| Hs.278627 \|\| AA633825 \|\| \|\| 51449□ | −3.439129051 | 0.15756 |
| 314820 \|\| \|\| Transcribed sequences \|\| Hs.122310 \|\| AI084343 \|\| \|\| □ | −3.438442625 | 0.13609 |
| 227011 \|\| P4HA2 \|\| procollagen-proline, 2-oxoglutarate 4-dioxygenase (proline 4- | −3.4345398 | 0.18984 |
| hydroxylase), alpha polypeptide \|\| \|\| Hs.104772 \|\| W49521 \|\| \|\| 8974□ | | |
| 109224 \|\| CTGF \|\| connective tissue growth factor \|\| Hs.410037 \|\| AA598794 \|\| \|\| 1490□ | −3.426414721 | 0.16550 |
| 226583 \|\| GUCY1A3 \|\| guanylate cyclase 1, soluble, alpha 3 \|\| Hs.433488 \|\| H19241 \|\| \|\| | −3.422070265 | 0.17023 |
| 2982□ | | |
| 113639 \|\| BLVRB \|\| **biliverdin reductase B (flavin reductase (NADPH)) \|\| Hs.76289 \|\| | −3.419694854 | 0.10650 |
| N76927 \|\| \|\| 645□ | | |
| 220664 \|\| MMP23B \|\| matrix metalloproteinase 23B \|\| Hs.211819 \|\| AA626131 \|\| \|\| | −3.395533507 | 0.17999 |
| 8510□ | | |
| 107459 \|\| NOTCH3 \|\| Notch homolog 3 (Drosophila) \|\| Hs.8546 \|\| T63511 \|\| \|\| 4854□ | −3.372003429 | 0.19396 |
| 99221 \|\| LGI2 \|\| leucine-rich repeat LGI family, member 2 \|\| Hs.12488 \|\| R42056 \|\| \|\| | −3.371173362 | 0.17256 |
| 55203□ | | |
| 111282 \|\| BGN \|\| biglycan \|\| Hs.821 \|\| R77226 \|\| \|\| 633□ | −3.366073175 | 0.13524 |
| 223440 \|\| MYO10 \|\| myosin X \|\| Hs.61638 \|\| AA700471 \|\| \|\| 4651□ | −3.359461493 | 0.17317 |
| 105810 \|\| MYH9 \|\| myosin, heavy polypeptide 9, non-muscle \|\| Hs.146550 \|\| T69926 \|\| \|\| | −3.346801153 | 0.21180 |
| 4627□ | | |
| 114768 \|\| \|\| \|\| \|\| N32201 \|\| \|\| □ | −3.346290803 | 0.15501 |
| 119739 \|\| KIAA2028 \|\| similar to PH (pleckstrin homology) domain \|\| Hs.255938 \|\| | −3.34566147 | 0.16760 |
| AA447583 \|\| \|\| 130271□ | | |
| 247373 \|\| ARK5 \|\| AMP-activated protein kinase family member 5 \|\| Hs.200598 \|\| | −3.337924955 | 0.19647 |
| R85685 \|\| EST \|\| 9891□ | | |
| 103965 \|\| BRE \|\| brain and reproductive organ-expressed (TNFRSF1A modulator) \|\| | −3.337409896 | 0.20577 |
| Hs.80426 \|\| AA477082 \|\| \|\| 9577□ | | |
| 223729 \|\| \|\| LOC388279 (LOC388279), mRNA \|\| Hs.237396 \|\| AA625672 \|\| \|\| 388279□ | −3.33570378 | 0.21452 |
| 221087 \|\| PRDC \|\| protein related to DAN and cerberus \|\| Hs.207407 \|\| AA778005 \|\| \|\| | −3.325186886 | 0.13837 |
| 64388□ | | |
| 119430 \|\| \|\| \|\| \|\| AI261207 \|\| \|\| □ | −3.319154507 | 0.21128 |
| 104616 \|\| PTN \|\| pleiotrophin (heparin binding growth factor 8, neurite growth-promoting | −3.31796017 | 0.13225 |
| factor 1) \|\| Hs.44 \|\| AA001449 \|\| \|\| 5764□ | | |
| 106115 \|\| GUCY1A3 \|\| guanylate cyclase 1, soluble, alpha 3 \|\| Hs.433488 \|\| H23049 \|\| \|\| | −3.291221417 | 0.19795 |
| 2982□ | | |
| 319834 \|\| \|\| \|\| \|\| AA910598 \|\| \|\| □ | −3.290266291 | 0.21454 |
| 311020 \|\| BACH2 \|\| BTB and CNC homology 1, basic leucine zipper transcription factor | −3.279195797 | 0.19824 |
| 2 \|\| Hs.88414 \|\| AI004995 \|\| \|\| 60468□ | | |
| 102662 \|\| LUM \|\| lumican \|\| Hs.406475 \|\| AA035657 \|\| \|\| 4060□ | −3.246726546 | 0.15174 |
| 107000 \|\| CSPG2 \|\| chondroitin sulfate proteoglycan 2 (versican) \|\| Hs.434488 \|\| | −3.246073578 | 0.17816 |
| AA722599 \|\| \|\| 1462□ | | |
| 309646 \|\| \|\| \|\| \|\| T64982 \|\| \|\| □ | −3.240061873 | 0.23339 |
| 109427 \|\| LRRN1 \|\| leucine rich repeat neuronal 1 \|\| Hs.126085 \|\| AA176867 \|\| \|\| | −3.227819818 | 0.17519 |
| 57633□ | | |
| 225959 \|\| COG8 \|\| component of oligomeric golgi complex 8 \|\| Hs.130849 \|\| AA864323 \|\| | −3.215227551 | 0.24215 |
| \|\| 84342□ | | |
| 112328 \|\| \|\| Clone IMAGE: 4791553, mRNA \|\| Hs.22907 \|\| R56764 \|\| \|\| □ | −3.206200397 | 0.20389 |
| 110722 \|\| LUM \|\| lumican \|\| Hs.406475 \|\| AA453712 \|\| \|\| 4060□ | −3.204634795 | 0.21578 |
| 109139 \|\| TAGLN2 \|\| transgelin 2 \|\| Hs.406504 \|\| H08563 \|\| \|\| 8407□ | −3.203785645 | 0.19909 |
| 107552 \|\| PMP22 \|\| peripheral myelin protein 22 \|\| Hs.372031 \|\| R26732 \|\| \|\| 5376□ | −3.185783651 | 0.24376 |
| 224335 \|\| LOC83468 \|\| gycosyltransferase \|\| Hs.159993 \|\| AA628462 \|\| \|\| 83468□ | −3.184917385 | 0.20385 |
| 114977 \|\| GPR48 \|\| G protein-coupled receptor 48 \|\| Hs.160271 \|\| H88521 \|\| \|\| 55366□ | −3.179562289 | 0.20866 |
| 103604 \|\| COL6A2 \|\| collagen, type VI, alpha 2 \|\| Hs.420269 \|\| AA633747 \|\| \|\| 1292□ | −3.177426303 | 0.20793 |
| 118489 \|\| MEOX2 \|\| mesenchyme homeo box 2 (growth arrest-specific homeo box) \|\| | −3.175758915 | 0.18480 |
| Hs.77858 \|\| H25223 \|\| \|\| 4223□ | | |
| 103475 \|\| RUNX2 \|\| runt-related transcription factor 2 \|\| Hs.122116 \|\| AA125922 \|\| \|\| | −3.173575994 | 0.20190 |
| 860□ | | |
| 117526 \|\| D2S448 \|\| Melanoma associated gene \|\| Hs.118893 \|\| W72043 \|\| \|\| 7837□ | −3.173145076 | 0.23589 |
| 110533 \|\| RAB31 \|\| RAB31, member RAS oncogene family \|\| Hs.223025 \|\| N72559 \|\| \|\| | −3.170393793 | 0.16266 |
| 11031□ | | |
| 307674 \|\| \|\| Transcribed sequence with strong similarity to protein pir: I56326 | −3.162232993 | 0.20188 |
| (H. sapiens) I56326 fatty acid binding protein homolog - human \|\| Hs.502696 \|\| AI359037 | | |
| \|\| \|\| □ | | |
| 111738 \|\| OSBPL3 \|\| oxysterol binding protein-like 3 \|\| Hs.197955 \|\| N24076 \|\| \|\| 26031□ | −3.161920118 | 0.22985 |
| 116127 \|\| \|\| \|\| \|\| AA975922 \|\| \|\| □ | −3.147895835 | 0.19461 |
| 117971 \|\| NAV1 \|\| neuron navigator 1 \|\| Hs.6298 \|\| AA043878 \|\| \|\| 89796□ | −3.138254927 | 0.20922 |
| 225591 \|\| LOC253827 \|\| hypothetical protein LOC253827 \|\| Hs.339024 \|\| AA663730 \|\| \|\| | −3.133486895 | 0.17730 |
| 253827□ | | |
| 120206 \|\| C5orf13 \|\| chromosome 5 open reading frame 13 \|\| Hs.508741 \|\| AA284280 \|\| | −3.132539485 | 0.22785 |
| \|\| 9315□ | | |
| 221015 \|\| \|\| Transcribed sequences \|\| Hs.480837 \|\| AA436194 \|\| \|\| □ | −3.121223727 | 0.05573 |
| 110275 \|\| TMEM16D \|\| transmembrane protein 16D \|\| Hs.58785 \|\| T67161 \|\| \|\| 121601□ | −3.120936994 | 0.18281 |
| 107787 \|\| ADAMTS1 \|\| a disintegrin-like and metalloprotease (reprolysin type) with | −3.113124284 | 0.20902 |
| thrombospondin type 1 motif, 1 \|\| Hs.8230 \|\| AA057170 \|\| \|\| 9510□ | | |
| 111105 \|\| COL6A1 \|\| collagen, type VI, alpha 1 \|\| Hs.415997 \|\| AA046525 \|\| \|\| 1291□ | −3.102915404 | 0.18491 |
| 311037 \|\| MMP11 \|\| matrix metalloproteinase 11 (stromelysin 3) \|\| Hs.143751 \|\| | −3.101674676 | 0.16853 |
| AA954935 \|\| \|\| 4320□ | | |
| 114455 \|\| SHC2 \|\| SHC (Src homology 2 domain containing) transforming protein 2 \|\| | −3.095612272 | 0.20863 |
| Hs.30965 \|\| H10072 \|\| \|\| 25759□ | | |
| 220332 \|\| DKFZp434L142 \|\| hypothetical protein DKFZp434L142 \|\| Hs.323583 \|\| | −3.084479618 | 0.20965 |
| AA490585 \|\| \|\| 51313□ | | |
| 98353 \|\| CREB3L1 \|\| cAMP responsive element binding protein 3-like 1 \|\| Hs.405961 \|\| | −3.079770224 | 0.15433 |
| T78909 \|\| \|\| 90993□ | | |
| 106501 \|\| FLJ38101 \|\| hypothetical protein FLJ38101 \|\| Hs.138563 \|\| W73382 \|\| \|\| | −3.073544558 | 0.21836 |
| 255919□ | | |
| 246546 \|\| PTGER3 \|\| prostaglandin E receptor 3 (subtype EP3) \|\| Hs.527970 \|\| | −3.06621108 | 0.18195 |
| AA151583 \|\| Prostaglandin E receptor 3 (subtype EP3) {alternative produc \|\| 5733□ | | |
| 103875 \|\| NRP2 \|\| neuropilin 2 \|\| Hs.368746 \|\| AA490279 \|\| \|\| 8828□ | −3.063320417 | 0.22330 |
| 103849 \|\| NR4A1 \|\| nuclear receptor subfamily 4, group A, member 1 \|\| Hs.1119 \|\| | −3.061983784 | 0.21322 |
| N94487 \|\| Nak1 = TR3 orphan receptor = homologue of Nur77/NGIF-B/N10 anti- \|\| 3164□ | | |
| 103922 \|\| \|\| \|\| \|\| N69764 \|\| \|\| □ | −3.050136135 | 0.23050 |
| 117232 \|\| KRT25A \|\| keratin 25A \|\| Hs.55412 \|\| W73634 \|\| \|\| 147183□ | −3.042902743 | 0.24115 |
| 107562 \|\| RHOBTB1 \|\| Rho-related BTB domain containing 1 \|\| Hs.15099 \|\| AA182796 \|\| | −3.033762311 | 0.21515 |
| \|\| 9886□ | | |
| 109084 \|\| LOX \|\| lysyl oxidase \|\| Hs.102267 \|\| H99075 \|\| \|\| 4015□ | −3.033504566 | 0.21765 |
| 100530 \|\| PDGFRL \|\| **platelet-derived growth factor receptor-like \|\| Hs.170040 \|\| | −3.030390634 | 0.23119 |
| AA461197 \|\| \|\| 5157□ | | |
| 110389 \|\| PLAU \|\| plasminogen activator, urokinase \|\| Hs.77274 \|\| AA284668 \|\| \|\| 5328□ | −3.028819781 | 0.21152 |
| 118511 \|\| CTGF \|\| connective tissue growth factor \|\| Hs.410037 \|\| AA044993 \|\| \|\| 1490□ | −3.021578292 | 0.18152 |
| 102234 \|\| INHBA \|\| inhibin, beta A (activin A, activin AB alpha polypeptide) \|\| Hs.28792 \|\| | −3.01977317 | 0.19859 |
| N27159 \|\| \|\| 3624□ | | |
| 316006 \|\| MAFB \|\| v-maf musculoaponeurotic fibrosarcoma oncogene homolog B | −3.018405929 | 0.22365 |
| (avian) \|\| Hs.169487 \|\| AA037402 \|\| \|\| 9935□ | | |
| 220522 \|\| FZD1 \|\| frizzled homolog 1 (Drosophila) \|\| Hs.94234 \|\| AA664127 \|\| \|\| 8321□ | −3.010872056 | 0.23073 |
| 223435 \|\| PLXNA2 \|\| plexin A2 \|\| Hs.350065 \|\| W74801 \|\| \|\| 5362□ | −2.996948469 | 0.20854 |
| 109049 \|\| IL1R1 \|\| interleukin 1 receptor, type I \|\| Hs.82112 \|\| R56687 \|\| IL-1 receptor | −2.989326964 | 0.22360 |
| type I \|\| 3554□ | | |
| 120205 \|\| LHFP \|\| lipoma HMGIC fusion partner \|\| Hs.93765 \|\| N58145 \|\| \|\| 10186□ | −2.988081349 | 0.22044 |
| 320321 \|\| WISP1 \|\| WNT1 inducible signaling pathway protein 1 \|\| Hs.194680 \|\| | −2.988070889 | 0.20683 |
| AI473336 \|\| \|\| 8840□ | | |
| 116729 \|\| TMEFF1 \|\| transmembrane protein with EGF-like and two follistatin-like | −2.979041186 | 0.23286 |
| domains 1 \|\| Hs.336224 \|\| AA431475 \|\| \|\| 8577□ | | |
| 119877 \|\| GALNTL2 \|\| UDP-N-acetyl-alpha-D-galactosamine:polypeptide N- | −2.977439434 | 0.12913 |
| acetylgalactosaminyltransferase-like 2 \|\| Hs.411308 \|\| AA055179 \|\| \|\| 117248□ | | |
| 111323 \|\| EGR1 \|\| early growth response 1 \|\| Hs.326035 \|\| AA486533 \|\| \|\| 1958□ | −2.976456034 | 0.20860 |
| 115196 \|\| LOX \|\| lysyl oxidase \|\| Hs.102267 \|\| W70343 \|\| \|\| 4015□ | −2.971254727 | 0.25088 |
| 331171 \|\| ADAMTS2 \|\| a disintegrin-like and metalloprotease (reprolysin type) with | −2.967818966 | 0.20671 |
| thrombospondin type 1 motif, 2 \|\| Hs.120330 \|\| AI624388 \|\| \|\| 9509□ | | |
| 115147 \|\| SOCS3 \|\| suppressor of cytokine signaling 3 \|\| Hs.436943 \|\| T72915 \|\| \|\| | −2.963262297 | 0.23401 |
| 9021□ | | |
| 117706 \|\| COL6A1 \|\| collagen, type VI, alpha 1 \|\| Hs.415997 \|\| AA047208 \|\| \|\| 1291□ | −2.957428728 | 0.22494 |
| 101017 \|\| C7orf10 \|\| chromosome 7 open reading frame 10 \|\| Hs.114611 \|\| N99256 \|\| \|\| | −2.953025076 | 0.19817 |
| 79783□ | | |
| 99904 \|\| CTSK \|\| cathepsin K (pycnodysostosis) \|\| Hs.83942 \|\| R00859 \|\| \|\| 1513□ | −2.948001872 | 0.19646 |
| 113419 \|\| PTGER3 \|\| prostaglandin E receptor 3 (subtype EP3) \|\| Hs.527970 \|\| | −2.943145972 | 0.14467 |
| AA406362 \|\| \|\| 5733□ | | |
| 226659 \|\| COBLL1 \|\| COBL-like 1 \|\| Hs.443943 \|\| AA099593 \|\| \|\| 22837□ | −2.940520116 | 0.23699 |
| 117179 \|\| DPP4 \|\| dipeptidylpeptidase 4 (CD26, adenosine deaminase complexing | −2.931900272 | 0.17796 |
| protein 2) \|\| Hs.44926 \|\| W70233 \|\| \|\| 1803□ | | |
| 101059 \|\| KIAA1295 \|\| KIAA1295 \|\| Hs.26204 \|\| AA131537 \|\| \|\| 57517□ | −2.928740012 | 0.23597 |
| 106224 \|\| KIT \|\| v-kit Hardy-Zuckerman 4 feline sarcoma viral oncogene homolog \|\| | −2.926367812 | 0.16784 |
| Hs.81665 \|\| N24824 \|\| \|\| 3815□ | | |
| 111147 \|\| RARRES2 \|\| retinoic acid receptor responder (tazarotene induced) 2 \|\| | −2.914514327 | 0.20850 |
| Hs.37682 \|\| AA481944 \|\| \|\| 5919□ | | |
| 99232 \|\| FN1 \|\| fibronectin 1 \|\| Hs.418138 \|\| W84711 \|\| \|\| 2335□ | −2.909556474 | 0.23344 |
| 222950 \|\| CACNA2D3 \|\| calcium channel, voltage-dependent, alpha 2/delta 3 subunit \|\| | −2.881078064 | 0.16052 |
| Hs.435112 \|\| R20288 \|\| \|\| 55799□ | | |
| 108970 \|\| PLA2R1 \|\| phospholipase A2 receptor 1, 180 kDa \|\| Hs.410477 \|\| W04525 \|\| \|\| | −2.881038261 | 0.23343 |
| 22925□ | | |
| 115793 \|\| \|\| \|\| \|\| W93592 \|\| \|\| □ | −2.876610653 | 0.18513 |
| 319567 \|\| FLJ12505 \|\| hypothetical protein FLJ12505 \|\| Hs.96885 \|\| AI280215 \|\| \|\| | −2.876266965 | 0.24913 |
| 79805□ | | |
| 105221 \|\| \|\| CDNA FLJ42250 fis, clone TKIDN2007828 \|\| Hs.22247 \|\| AA455087 \|\| \|\| □ | −2.874501278 | 0.20673 |
| 113522 \|\| SMPDL3A \|\| sphingomyelin phosphodiesterase, acid-like 3A \|\| Hs.277962 \|\| | −2.87117851 | 0.21210 |
| AA676836 \|\| \|\| 10924□ | | |
| 104275 \|\| FN1 \|\| fibronectin 1 \|\| Hs.418138 \|\| N26285 \|\| \|\| 2335□ | −2.863144109 | 0.22540 |
| 314465 \|\| CDC42EP3 \|\| CDC42 effector protein (Rho GTPase binding) 3 \|\| Hs.352554 \|\| | −2.845562741 | 0.23353 |
| AA708976 \|\| \|\| 10602□ | | |
| 116230 \|\| SGCE \|\| sarcoglycan, epsilon \|\| Hs.409798 \|\| AA432066 \|\| \|\| 8910□ | −2.844791355 | 0.26047 |
| 119105 \|\| TUB \|\| tubby homolog (mouse) \|\| Hs.54468 \|\| AI261674 \|\| \|\| 7275□ | −2.843264569 | 0.22966 |
| 102254 \|\| DPYSL3 \|\| dihydropyrimidinase-like 3 \|\| Hs.150358 \|\| AI831083 \|\| \|\| 1809□ | −2.841135506 | 0.25800 |
| 105408 \|\| SDC2 \|\| syndecan 2 (heparan sulfate proteoglycan 1, cell surface-associated, | −2.83654257 | 0.22038 |
| fibroglycan) \|\| Hs.1501 \|\| H64346 \|\| \|\| 6383□ | | |
| 116443 \|\| FBN1 \|\| fibrillin 1 (Marfan syndrome) \|\| Hs.750 \|\| AA418674 \|\| \|\| 2200□ | −2.835028335 | 0.17789 |
| 113891 \|\| \|\| **Transcribed sequence with weak similarity to protein sp: P39188 | −2.822658193 | 0.25979 |
| (H. sapiens) ALU1_HUMAN Alu subfamily J sequence contamination warning entry \|\| | | |
| Hs.444835 \|\| H96672 \|\| \|\| □ | | |
| 102960 \|\| ZNF515 \|\| zinc finger protein 515 \|\| Hs.436767 \|\| H68285 \|\| \|\| 169792□ | −2.818669312 | 0.21639 |
| 98915 \|\| OGN \|\| osteoglycin (osteoinductive factor, mimecan) \|\| Hs.109439 \|\| AA045327 | −2.81433711 | 0.19730 |
| \|\| \|\| 4969□ | | |
| 119660 \|\| MGC39325 \|\| hypothetical protein MGC39325 \|\| Hs.34054 \|\| R86669 \|\| \|\| | −2.800857757 | 0.23672 |
| 90362□ | | |
| 104139 \|\| HAK \|\| heart alpha-kinase \|\| Hs.388674 \|\| R27572 \|\| \|\| 115701□ | −2.799618256 | 0.26074 |
| 220989 \|\| \|\| \|\| \|\| R20393 \|\| \|\| □ | −2.791031277 | 0.17897 |
| 108645 \|\| COPEB \|\| core promoter element binding protein \|\| Hs.285313 \|\| AA013481 \|\| | −2.788222856 | 0.24745 |
| \|\| 1316□ | | |
| 117533 \|\| \|\| \|\| \|\| AI049923 \|\| \|\| □ | −2.783132557 | 0.24676 |
| 104601 \|\| ITGA10 \|\| integrin, alpha 10 \|\| Hs.158237 \|\| H43656 \|\| \|\| 8515□ | −2.774008391 | 0.22260 |
| 226084 \|\| C9orf88 \|\| **chromosome 9 open reading frame 88 \|\| Hs.109857 \|\| N34466 \|\| | −2.772499659 | 0.26369 |
| \|\| 64855□ | | |
| 220157 \|\| LOC253827 \|\| hypothetical protein LOC253827 \|\| Hs.339024 \|\| N91811 \|\| \|\| | −2.768601634 | 0.17649 |
| 253827□ | | |
| 107770 \|\| \|\| CDNA FLJ43362 fis, clone NT2RP7017365 \|\| Hs.209253 \|\| AA410454 \|\| \|\| □ | −2.765331743 | 0.23660 |
| 307579 \|\| \|\| Full length insert cDNA clone ZD77F06 \|\| Hs.124863 \|\| AA975413 \|\| \|\| □ | −2.76278685 | 0.24212 |
| 107204 \|\| FLJ14525 \|\| hypothetical protein FLJ14525 \|\| Hs.26812 \|\| AA464028 \|\| \|\| | −2.760471326 | 0.20615 |
| 84886□ | | |
| 224528 \|\| ROR2 \|\| receptor tyrosine kinase-like orphan receptor 2 \|\| Hs.208080 \|\| | −2.757205189 | 0.26958 |
| AA701961 \|\| \|\| 4920□ | | |
| 118589 \|\| \|\| CDNA: FLJ22256 fis, clone HRC02860 \|\| Hs.288741 \|\| H71070 \|\| \|\| □ | −2.756218606 | 0.18991 |
| 101022 \|\| TTLL4 \|\| **tubulin tyrosine ligase-like family, member 4 \|\| Hs.169910 \|\| | −2.755712581 | 0.27418 |
| AA463454 \|\| \|\| 9654□ | | |
| 120504 \|\| PLXDC2 \|\| plexin domain containing 2 \|\| Hs.444997 \|\| AA166917 \|\| \|\| 84898□ | −2.754197706 | 0.23788 |
| 118366 \|\| COL5A1 \|\| collagen, type V, alpha 1 \|\| Hs.528321 \|\| R75635 \|\| \|\| 1289□ | −2.740772973 | 0.14987 |
| 308090 \|\| CYR61 \|\| cysteine-rich, angiogenic inducer, 61 \|\| Hs.8867 \|\| AA012892 \|\| \|\| | −2.73496564 | 0.24782 |
| 3491□ | | |
| 312629 \|\| CDKN1C \|\| cyclin-dependent kinase inhibitor 1C (p57, Kip2) \|\| Hs.106070 \|\| | −2.731401675 | 0.24442 |
| AI676118 \|\| \|\| 1028□ | | |
| 116073 \|\| ASAM \|\| adipocyte-specific adhesion molecule \|\| Hs.135121 \|\| R63922 \|\| \|\| | −2.730434425 | 0.28364 |
| 79827□ | | |
| 102206 \|\| \|\| MRNA; cDNA DKFZp564O0862 (from clone DKFZp564O0862) \|\| Hs.99472 | −2.730341175 | 0.25253 |
| \|\| R16259 \|\| \|\| □ | | |
| 308073 \|\| NAV1 \|\| neuron navigator 1 \|\| Hs.6298 \|\| AA010927 \|\| \|\| 89796□ | −2.726200933 | 0.28149 |
| 224275 \|\| AGTRL1 \|\| angiotensin \|\| receptor-like 1 \|\| Hs.438311 \|\| R58969 \|\| \|\| 187□ | −2.726002894 | 0.24533 |
| 223769 \|\| RASEF \|\| RAS and EF hand domain containing \|\| Hs.165464 \|\| AA826324 \|\| \|\| | −2.724862332 | 0.19740 |
| 158158□ | | |
| 313561 \|\| FZD2 \|\| frizzled homolog 2 (Drosophila) \|\| Hs.142912 \|\| AI279304 \|\| \|\| 2535□ | −2.722940565 | 0.22626 |
| 226524 \|\| FLJ25348 \|\| hypothetical protein FLJ25348 \|\| Hs.62604 \|\| AA772494 \|\| \|\| | −2.718939066 | 0.24470 |
| 90853□ | | |
| 103073 \|\| GEM \|\| GTP binding protein overexpressed in skeletal muscle \|\| Hs.79022 \|\| | −2.71153179 | 0.26434 |
| AA418077 \|\| \|\| 2669□ | | |
| 119427 \|\| FLJ38508 \|\| hypothetical protein FLJ38508 \|\| Hs.321988 \|\| AA424371 \|\| \|\| | −2.711399933 | 0.25938 |
| 160428□ | | |
| 310609 \|\| ZIC1 \|\| Zic family member 1 (odd-paired homolog, Drosophila) \|\| Hs.41154 \|\| | −2.700261083 | 0.22824 |
| AI358916 \|\| \|\| 7545□ | | |
| 309270 \|\| CYR61 \|\| cysteine-rich, angiogenic inducer, 61 \|\| Hs.8867 \|\| AI014487 \|\| \|\| | −2.697503545 | 0.23085 |
| 3491□ | | |
| 222117 \|\| CDC42EP3 \|\| CDC42 effector protein (Rho GTPase binding) 3 \|\| Hs.352554 \|\| | −2.692044813 | 0.22101 |
| AA213816 \|\| \|\| 10602□ | | |
| 112655 \|\| SEPT11 \|\| septin 11 \|\| Hs.386784 \|\| AA428368 \|\| \|\| 55752□ | −2.69181035 | 0.24131 |
| 113586 \|\| FBN2 \|\| fibrillin 2 (congenital contractural arachnodactyly) \|\| Hs.79432 \|\| | −2.691012246 | 0.26154 |
| N74178 \|\| \|\| 2201□ | | |
| 108846 \|\| TNC \|\| tenascin C (hexabrachion) \|\| Hs.98998 \|\| R39239 \|\| \|\| 3371□ | −2.685339774 | 0.17512 |
| 223897 \|\| AXIN2 \|\| axin 2 (conductin, axil) \|\| Hs.127337 \|\| R33823 \|\| \|\| 8313□ | −2.670642811 | 0.27258 |
| 113367 \|\| LRIG1 \|\| leucine-rich repeats and immunoglobulin-like domains 1 \|\| Hs.528353 | −2.670189288 | 0.27588 |
| \|\| AA159578 \|\| \|\| 26018□ | | |
| 112938 \|\| ID4 \|\| inhibitor of DNA binding 4, dominant negative helix-loop-helix protein \|\| | −2.667062386 | 0.16000 |
| Hs.391392 \|\| AA452493 \|\| \|\| 3400□ | | |
| 118921 \|\| PITX2 \|\| paired-like homeodomain transcription factor 2 \|\| Hs.92282 \|\| T64905 | −2.664895268 | 0.23432 |
| \|\| \|\| 5308□ | | |
| 102191 \|\| CRABP2 \|\| cellular retinoic acid binding protein 2 \|\| Hs.183650 \|\| AA598508 \|\| | −2.657360356 | 0.30629 |
| \|\| 1382□ | | |
| 310726 \|\| FLJ10312 \|\| hypothetical protein FLJ10312 \|\| Hs.183114 \|\| AA918229 \|\| \|\| | −2.649487207 | 0.28821 |
| 79822□ | | |
| 112105 \|\| DSP \|\| desmoplakin \|\| Hs.349499 \|\| R33456 \|\| \|\| 1832□ | −2.640137304 | 0.27411 |
| 98823 \|\| CDH11 \|\| cadherin 11, type 2, OB-cadherin (osteoblast) \|\| Hs.443435 \|\| H96738 | −2.637823733 | 0.29192 |
| \|\| \|\| 1009□ | | |
| 309548 \|\| \|\| Transcribed sequence with moderate similarity to protein pdb: 1LBG (E. coli) | −2.620334775 | 0.27234 |
| B Chain B, Lactose Operon Repressor Bound To 21-Base Pair Symmetric Operator | | |
| Dna, Alpha Carbons Only \|\| Hs.433695 \|\| AA488444 \|\| \|\| □ | | |
| 99539 \|\| TBX15 \|\| T-box 15 \|\| Hs.164680 \|\| AA463229 \|\| \|\| 6913□ | −2.619375394 | 0.19221 |
| 308735 \|\| CALD1 \|\| caldesmon 1 \|\| Hs.443811 \|\| AA453669 \|\| \|\| 800□ | −2.617482873 | 0.30116 |
| 307146 \|\| AXIN2 \|\| axin 2 (conductin, axil) \|\| Hs.127337 \|\| AA976642 \|\| \|\| 8313□ | −2.607126029 | 0.25101 |
| 224633 \|\| FSTL3 \|\| **follistatin-like 3 (secreted glycoprotein) \|\| Hs.433827 \|\| AA417274 \|\| | −2.604061305 | 0.26719 |
| \|\| 10272□ | | |
| 310488 \|\| TNC \|\| tenascin C (hexabrachion) \|\| Hs.98998 \|\| AA598955 \|\| \|\| 3371□ | −2.597884862 | 0.21166 |
| 222592 \|\| UCC1 \|\| upregulated in colorectal cancer gene 1 \|\| Hs.46721 \|\| N47444 \|\| \|\| | −2.575424669 | 0.28436 |
| 54749□ | | |
| 311649 \|\| ASAM \|\| adipocyte-specific adhesion molecule \|\| Hs.135121 \|\| AI038014 \|\| \|\| | −2.569584439 | 0.29617 |
| 79827□ | | |
| 103460 \|\| GPNMB \|\| glycoprotein (transmembrane) nmb \|\| Hs.389964 \|\| AA059346 \|\| \|\| | −2.552580313 | 0.31323 |
| 10457□ | | |
| 119864 \|\| TGFB2 \|\| transforming growth factor, beta 2 \|\| Hs.169300 \|\| N48082 \|\| TGF | −2.550178141 | 0.28381 |
| beta-2 \|\| 7042□ | | |
| 112254 \|\| FMOD \|\| fibromodulin \|\| Hs.442844 \|\| AA485748 \|\| \|\| 2331□ | −2.544653315 | 0.27243 |
| 116618 \|\| LOC345667 \|\| similar to ADAMTS-10 precursor (A disintegrin and | −2.538740128 | 0.20078 |
| metalloproteinase with thrombospondin motifs 10) (ADAM-TS 10) (ADAM-TS10) \|\| | | |
| Hs.382857 \|\| W94295 \|\| \|\| 345667□ | | |
| 163376 \|\| NRG1 \|\| neuregulin 1 \|\| Hs.172816 \|\| H24357 \|\| Heregulin alpha = neu = glial | −2.537673426 | 0.26857 |
| growth factor 2 \|\| 3084□ | | |
| 220142 \|\| GALNT5 \|\| UDP-N-acetyl-alpha-D-galactosamine: polypeptide N- | −2.536011051 | 0.31037 |
| acetylgalactosaminyltransferase 5 (GalNAc-T5) \|\| Hs.443716 \|\| AA676660 \|\| \|\| 11227□ | | |
| 118371 \|\| NRG1 \|\| neuregulin 1 \|\| Hs.172816 \|\| R72075 \|\| \|\| 3084□ | −2.533343588 | 0.28387 |
| 101570 \|\| \|\| Full length insert cDNA YN73H08 \|\| Hs.120725 \|\| H51425 \|\| \|\| □ | −2.532334328 | 0.29882 |
| 119852 \|\| CTSE \|\| cathepsin E \|\| Hs.1355 \|\| H94487 \|\| \|\| 1510□ | −2.523818963 | 0.27918 |
| 98814 \|\| FLJ38507 \|\| colon carcinoma related protein \|\| Hs.435013 \|\| H02837 \|\| \|\| | −2.500720774 | 0.29184 |
| 389136□ | | |
| 106045 \|\| PDIR \|\| for protein disulfide isomerase-related \|\| Hs.76901 \|\| AA404387 \|\| \|\| | −2.499096236 | 0.30661 |
| 10954□ | | |
| 314096 \|\| HEY2 \|\| hairy/enhancer-of-split related with YRPW motif 2 \|\| Hs.144287 \|\| | −2.494859741 | 0.24965 |
| AI299482 \|\| \|\| 23493□ | | |
| 313144 \|\| PART1 \|\| prostate androgen-regulated transcript 1 \|\| Hs.412792 \|\| AI311391 \|\| | −2.491715872 | 0.27052 |
| \|\| 25859□ | | |
| 221911 \|\| ADAMTS9 \|\| a disintegrin-like and metalloprotease (reprolysin type) with | −2.486715255 | 0.29988 |
| thrombospondin type 1 motif, 9 \|\| Hs.318751 \|\| AI002071 \|\| \|\| 56999□ | | |
| 116816 \|\| THBS1 \|\| thrombospondin 1 \|\| Hs.164226 \|\| AA232645 \|\| \|\| 7057□ | −2.481503988 | 0.21943 |
| 225802 \|\| C6orf65 \|\| chromosome 6 open reading frame 65 \|\| Hs.47403 \|\| R38266 \|\| \|\| | −2.478661923 | 0.24365 |
| 221336□ | | |
| 118685 \|\| KIAA0367 \|\| KIAA0367 \|\| Hs.23311 \|\| AA447773 \|\| \|\| 23273□ | −2.475710061 | 0.27948 |
| 105666 \|\| ELL2 \|\| elongation factor, RNA polymerase II, 2 \|\| Hs.192221 \|\| AA284232 \|\| \|\| | −2.473398269 | 0.32797 |
| 22936□ | | |
| 117476 \|\| FGF12 \|\| fibroblast growth factor 12 \|\| Hs.343809 \|\| N71102 \|\| FGF- | −2.470929198 | 0.31253 |
| 12 = Fibroblast growth factor-12 \|\| 2257□ | | |
| 226512 \|\| K5B \|\| keratin 5b \|\| Hs.121824 \|\| AA775536 \|\| \|\| 196374□ | −2.467155522 | 0.26635 |
| 98589 \|\| E2IG4 \|\| hypothetical protein, estradiol-induced \|\| Hs.8361 \|\| R13844 \|\| \|\| | −2.459862586 | 0.31167 |
| 25987□ | | |
| 101588 \|\| PDGFRL \|\| **platelet-derived growth factor receptor-like \|\| Hs.170040 \|\| | −2.451931374 | 0.32001 |
| AA454868 \|\| PDGF receptor beta-like tumor suppressor \|\| 5157□ | | |
| 101528 \|\| RGS7 \|\| regulator of G-protein signalling 7 \|\| Hs.79348 \|\| H23046 \|\| | −2.427067503 | 0.13602 |
| BL34 = RGS1 = regulator of G-protein signaling which inhibits SD \|\| 6000□ | | |
| 224067 \|\| \|\| \|\| \|\| AA703557 \|\| \|\| □ | −2.424238673 | 0.30920 |
| 223256 \|\| DOCK6 \|\| dedicator of cytokinesis 6 \|\| Hs.8982 \|\| AA678373 \|\| \|\| 57572□ | −2.41561796 | 0.32156 |
| 310856 \|\| \|\| Similar to RIKEN cDNA 2210021J22 (LOC150383), mRNA \|\| Hs.275711 \|\| | −2.409260788 | 0.31477 |
| R15978 \|\| \|\| 84730□ | | |
| 308336 \|\| KIAA0934 \|\| KIAA0934 \|\| Hs.116204 \|\| AA410299 \|\| \|\| 22982□ | −2.409156873 | 0.31484 |
| 110348 \|\| CALD1 \|\| caldesmon 1 \|\| Hs.443811 \|\| H51958 \|\| \|\| 800□ | −2.396952622 | 0.33105 |
| 103146 \|\| FLJ14525 \|\| hypothetical protein FLJ14525 \|\| Hs.26812 \|\| N59373 \|\| \|\| 84886□ | −2.392817438 | 0.27747 |
| 318583 \|\| \|\| \|\| \|\| AI334253 \|\| \|\| □ | −2.37526706 | 0.34925 |
| 222380 \|\| F2R \|\| coagulation factor II (thrombin) receptor \|\| Hs.128087 \|\| N20406 \|\| \|\| | −2.370837088 | 0.26567 |
| 2149□ | | |
| 100127 \|\| PTHLH \|\| parathyroid hormone-like hormone \|\| Hs.89626 \|\| AA845432 \|\| \|\| | −2.364020974 | 0.30800 |
| 5744□ | | |
| 115579 \|\| OGN \|\| osteoglycin (osteoinductive factor, mimecan) \|\| Hs.109439 \|\| | −2.355224287 | 0.16376 |
| AA219099 \|\| \|\| 4969□ | | |
| 103059 \|\| FLJ30277 \|\| hypothetical protein FLJ30277 \|\| Hs.182635 \|\| AA284282 \|\| \|\| | −2.331787111 | 0.28360 |
| 152641□ | | |
| 101694 \|\| AQP1 \|\| aquaporin 1 (channel-forming integral protein, 28 kDa) \|\| Hs.76152 \|\| | −2.33107841 | 0.29127 |
| H23036 \|\| \|\| 358□ | | |
| 224392 \|\| DAF \|\| decay accelerating factor for complement (CD55, Cromer blood group | −2.312540385 | 0.31095 |
| system) \|\| Hs.408864 \|\| AA678160 \|\| \|\| 1604□ | | |
| 114872 \|\| KIAA1906 \|\| KIAA1906 protein \|\| Hs.6496 \|\| R41754 \|\| \|\| 114795□ | −2.309392097 | 0.36974 |
| 111723 \|\| \|\| **CDNA FLJ37050 fis, clone BRACE2013369 \|\| Hs.144285 \|\| AA115848 \|\| | −2.304793381 | 0.34844 |
| \|\| □ | | |
| 99209 \|\| \|\| \|\| \|\| AA076645 \|\| \|\| □ | −2.299695167 | 0.28337 |
| 117659 \|\| THBS1 \|\| thrombospondin 1 \|\| Hs.164226 \|\| AA007557 \|\| \|\| 7057□ | −2.281442723 | 0.28275 |
| 319946 \|\| OPRS1 \|\| opioid receptor, sigma 1 \|\| Hs.24447 \|\| AI365014 \|\| \|\| 10280□ | −2.271513987 | 0.31710 |
| 330930 \|\| COL6A2 \|\| collagen, type VI, alpha 2 \|\| Hs.420269 \|\| AI830005 \|\| \|\| 1292□ | −2.270929943 | 0.35900 |
| 103549 \|\| CD9 \|\| CD9 antigen (p24) \|\| Hs.387579 \|\| AA412053 \|\| CD9 \|\| 928□ | −2.266185192 | 0.28075 |
| 119647 \|\| TDO2 \|\| tryptophan 2,3-dioxygenase \|\| Hs.183671 \|\| T72398 \|\| \|\| 6999□ | −2.263748791 | 0.13885 |
| 330857 \|\| GUCY1A3 \|\| guanylate cyclase 1, soluble, alpha 3 \|\| Hs.433488 \|\| AI954306 \|\| | −2.242248585 | 0.35200 |
| \|\| 2982□ | | |
| 111093 \|\| COL11A1 \|\| collagen, type XI, alpha 1 \|\| Hs.439168 \|\| R31701 \|\| \|\| 1301□ | −2.238183145 | 0.34714 |
| 316245 \|\| CH25H \|\| cholesterol 25-hydroxylase \|\| Hs.47357 \|\| AI081548 \|\| \|\| 9023□ | −2.23770944 | 0.38076 |
| 309421 \|\| GPRC5B \|\| G protein-coupled receptor, family C, group 5, member B \|\| | −2.237401434 | 0.34370 |
| Hs.448805 \|\| AI356028 \|\| \|\| 51704□ | | |
| 307778 \|\| ECM2 \|\| extracellular matrix protein 2, female organ and adipocyte specific \|\| | −2.231012008 | 0.31983 |
| Hs.117060 \|\| AI016683 \|\| \|\| 1842□ | | |
| 111508 \|\| MYL1 \|\| myosin, light polypeptide 1, alkali; skeletal, fast \|\| Hs.187338 \|\| | −2.229203269 | 0.10333 |
| AA196393 \|\| \|\| 4632□ | | |
| 117657 \|\| \|\| \|\| \|\| AA431770 \|\| \|\| □ | −2.224581366 | 0.36989 |
| 108941 \|\| SCYE1 \|\| small inducible cytokine subfamily E, member 1 (endothelial | −2.221621835 | 0.31986 |
| monocyte-activating) \|\| Hs.105656 \|\| H42360 \|\| \|\| 9255□ | | |
| 104646 \|\| CLECSF2 \|\| C-type (calcium dependent, carbohydrate-recognition domain) | −2.219979062 | 0.30121 |
| lectin, superfamily member 2 (activation-induced) \|\| Hs.85201 \|\| H11732 \|\| | | |
| AICL = activation-induced C-type lectin \|\| 9976□ | | |
| 106170 \|\| IL1R1 \|\| interleukin 1 receptor, type I \|\| Hs.82112 \|\| N69425 \|\| \|\| 3554□ | −2.211490527 | 0.30587 |
| 220524 \|\| NOV \|\| nephroblastoma overexpressed gene \|\| Hs.235935 \|\| AA910443 \|\| \|\| | −2.193099238 | 0.29683 |
| 4856□ | | |
| 109898 \|\| TGFB2 \|\| transforming growth factor, beta 2 \|\| Hs.169300 \|\| N45138 \|\| TGF | −2.187239483 | 0.33663 |
| beta-2 \|\| 7042□ | | |
| 224651 \|\| FLJ12505 \|\| hypothetical protein FLJ12505 \|\| Hs.96885 \|\| AA885478 \|\| \|\| | −2.180502753 | 0.27729 |
| 79805□ | | |
| 99237 \|\| DPT \|\| dermatopontin \|\| Hs.80552 \|\| R48303 \|\| \|\| 1805□ | −2.173432539 | 0.29185 |
| 226908 \|\| PLS3 \|\| plastin 3 (T isoform) \|\| Hs.430166 \|\| AA953747 \|\| \|\| 5358□ | −2.172311313 | 0.36982 |
| 109333 \|\| AMPH \|\| amphiphysin (Stiff-Man syndrome with breast cancer 128 kDa | −2.169133071 | 0.29085 |
| autoantigen) \|\| Hs.173034 \|\| H08504 \|\| \|\| 273□ | | |
| 117106 \|\| HLA-DQA2 \|\| **major histocompatibility complex, class II, DQ alpha 2 \|\| | −2.16700573 | 0.30873 |
| Hs.289095 \|\| T63324 \|\| \|\| 3118□ | | |
| 120362 \|\| AD-017 \|\| **glycosyltransferase AD-017 \|\| Hs.297304 \|\| H94897 \|\| \|\| 55830□ | −2.166465331 | 0.24037 |
| 247136 \|\| \|\| Similar to 40S ribosomal protein S26 (LOC391165), mRNA \|\| Hs.512517 \|\| | −2.162993691 | 0.36084 |
| W47595 \|\| Transforming growth factor beta 2 \|\| 391165□ | | |
| 118336 \|\| \|\| Clone DNA100312 VSSW1971 (UNQ1971) mRNA, complete cds \|\| | −2.160770821 | 0.30907 |
| Hs.437875 \|\| AA128017 \|\| \|\| □ | | |
| 315088 \|\| RARB \|\| retinoic acid receptor, beta \|\| Hs.436538 \|\| H69474 \|\| \|\| 5915□ | −2.160305599 | 0.34873 |
| 100990 \|\| COL16A1 \|\| collagen, type XVI, alpha 1 \|\| Hs.26208 \|\| R54778 \|\| \|\| 1307□ | −2.15424395 | 0.33398 |
| 115461 \|\| \|\| Transcribed sequence with weak similarity to protein sp: P39194 | −2.146653046 | 0.21535 |
| (H. sapiens) ALU7_HUMAN Alu subfamily SQ sequence contamination warning entry \|\| | | |
| Hs.270149 \|\| H77493 \|\| \|\| □ | | |
| 330437 \|\| MMP23B \|\| matrix metalloproteinase 23B \|\| Hs.211819 \|\| AW072298 \|\| \|\| | −2.135258655 | 0.39981 |
| 8510□ | | |
| 100028 \|\| CLECSF2 \|\| **C-type (calcium dependent, carbohydrate-recognition domain) | −2.134108945 | 0.30332 |
| lectin, superfamily member 2 (activation-induced) \|\| Hs.85201 \|\| AA417921 \|\| \|\| 9976□ | | |
| 309775 \|\| JDP2 \|\| jun dimerization protein 2 \|\| Hs.154095 \|\| AA932870 \|\| \|\| 122953□ | −2.126897294 | 0.39618 |
| 99380 \|\| CRIP2 \|\| cysteine-rich protein 2 \|\| Hs.70327 \|\| AA873604 \|\| \|\| 1397□ | −2.118673576 | 0.21869 |
| 312995 \|\| FLJ12505 \|\| hypothetical protein FLJ12505 \|\| Hs.96885 \|\| AI291607 \|\| \|\| | −2.116793914 | 0.31066 |
| 79805□ | | |
| 433344 \|\| \|\| *mitoch. cont. cellular retinoic acid-binding protein 2 \|\| \|\| \|\| \|\| □ | −2.105798653 | 0.40241 |
| 313394 \|\| HLF \|\| hepatic leukemia factor \|\| Hs.250692 \|\| R59192 \|\| \|\| 3131□ | −2.100244406 | 0.41924 |
| 110833 \|\| DKFZP434B172 \|\| DKFZP434B172 protein \|\| Hs.112822 \|\| AA610143 \|\| \|\| | −2.098184617 | 0.39592 |
| 26172□ | | |
| 115688 \|\| SEMA3D \|\| sema. domain, immunoglobulin domain (Ig), short basic domain, | −2.095344203 | 0.30079 |
| secreted, (semaphorin) 3D \|\| Hs.187319 \|\| AA165409 \|\| \|\| 223117□ | | |
| 223538 \|\| KIAA0992 \|\| palladin \|\| Hs.194431 \|\| AA705655 \|\| \|\| 23022□ | −2.086997639 | 0.34870 |

Overall survival (OS) was defined by death from any cause. In this cohort of young breast cancer patients, only six patients died of causes other than breast cancer (five second primaries and one cardiovascular). Distant metastasis-free survival (DMFS) was defined by a distant metastasis as a first recurrence event; data on all patients were censored on the date of the last follow-up visit, death from causes other than breast cancer, the recurrence of local or regional disease, or the development of a second primary cancer, including contra-lateral breast cancer. Kaplan-Meier survival curves were compared by the Cox-Mantel log-rank test in Winstat for Microsoft Excel (R. Fitch Software, Germany). Multivariate analysis by the Cox proportional hazard method was performed using the software package SPSS® 11.5 (SPSS, Inc.).

TMA construction. A TMA of fibroblastic conditions was constructed using a manual tissue arrayer (Beecher Instruments, Silver Spring, Md., United States) following previously described techniques with modifications. Briefly, certain specimens, such as skin and fistula tract, contained tissues whose positional orientation was important for analysis. Coring of these tissues could lose orientation of the cells within the core. Therefore, orientation-sensitive material was dissected from the original blocks and re-embedded into the paraffin block used for tissue arraying. Tissues thus embedded included skin, lung, breast, granulation tissue, and fistula tract. After the embedding process was completed, construction of the tissue array was performed using single 2-mm cores. In addition, the TMA contained 0.6-mm cores of lobular (n=14) and ductal (n=10) breast carcinomas, fibroadenomas (n=11), SFT (n=5), DTF (n=5), and colorectal carcinomas (n=2), scar (n=1), and keloid (n=1). All samples were obtained from archived material at the Stanford University Medical Center Department of Pathology between 2001 and 2004 with IRB approval. The cores were taken from areas in the paraffin block that were representative of the diagnostic tissue.

IHC. Serial sections of 4 μm were cut from the TMA blocks, deparaffinized in xylene, and hydrated in a graded series of alcohol. The slides were pretreated with citrate buffer and a microwave step. Staining was then performed using the DAKO EnVision+ System, Peroxidase (DAB), (DAKO, Cambridgeshire, United Kingdom) for APOD (Clone 36C6, 1:40 dilution, Novocastra, Newcastle, United Kingdom), CD34 (1:20 dilution, BD Biosciences, San Diego, Calif., United States), and BCL2 (1:800 dilution, DAKO Cytomation, Carpinteria, Calif., United States) stains. Results were interpreted as follows: Staining was interpreted as negative when no more than 5% of the spindled stromal cells showed light staining. A score of “weak positive” was given for light-brown staining in more than 5% of the spindled stromal cells. A score of “strong positive” was given for staining in more than 50% of the spindled stromal cells. Cores in which no diagnostic material was present were omitted from further analysis. The cores were initially reviewed independently by two pathologists (RW and MvdR), and disagreements were reviewed together to, achieve a consensus score. Scoring of the arrays was analyzed using the Deconvoluter software as previously described [24], with each sample receiving the highest score for either of the two cores.

In situ hybridization (ISH). ISH of TMA sections was performed based on a protocol published previously. Briefly, digoxigenin (DIG)-labeled sense and anti-sense RNA probes are generated by PCR amplification of 400 to 600 bp products with the T7 promoter incorporated into the primers. In vitro transcription was performed with a DIG RNA-labeling kit and T7 polymerase according to the manufacturer's protocol (Roche Diagnostics, Indianapolis, Ind., United States). We cut sections 4 μm thick from the paraffin blocks, deparaffinized them in xylene, and hydrated them in graded concentrations of ethanol for 5 min each. Sections were then incubated with 3% hydrogen peroxide, followed by digestion in 10 μg/ml of proteinase K at 37° C. for 30 min. Sections were hybridized overnight at 55° C. with either sense or anti-sense riboprobes at 150 ng/ml dilution in mRNA hybridization buffer (DAKO). The following day, sections were washed in 2×SSC and incubated with a 1:35 dilution of RNase A cocktail (Ambion, Austin, Tex., United States) in 2×SSC for 30 min at 37° C. Next, sections were stringently washed in 2×SSC/50% formamide twice, followed by one wash at 0.08×SSC at 50° C. Biotin blocking reagents (DAKO) were applied to the section to block the endogenous biotin. For signal amplification, a HRP-conjugated rabbit anti-DIG antibody (DAKO) was used to catalyze the deposition of biotinyl tyramide, followed by secondary streptavidin complex (GenPoint kit; DAKO). The final signal was developed with DAB (GenPoint kit; DAKO), and the tissues were counterstained in hematoxylin for 15 s.

### EXAMPLE 2

### Analysis of Ovarian Cancer

Using the datasets of Example 1, 23 ovarian serous carcinomas and serous neoplasms of low malignant potential were clustered based on our fibroblast gene list from the DTF and SFT reference datasets. The results how a similar classification to that found for breast carcinoma, indicating the underlying similarity of tumor-associated stromal cells even where the carcinoma cells are unrelated.

### EXAMPLE 3

### The Gene Expression Profile of Extraskeletal Myxoid Chondrosarcoma

Extraskeletal myxoid chondrosarcoma (EMC) is a soft tissue tumour that occurs primarily in the extremities and is characterized by a balanced translocation most commonly involving t(9;22) (q22;q12). The morphological spectrum of EMC is broad and thus a diagnosis based on histology alone can be difficult. Currently, no systemic therapy exists that improves survival in patients with EMC. In the present study, gene expression profiling has been performed to discover new diagnostic markers and potential therapeutic targets for this tumour type. Global gene expression profiling of ten EMCs and 26 other sarcomas using 42 000 spot cDNA microarrays revealed that the cases of EMC were closely related to each other and distinct from the other tumours profiled. Significance analysis of microarrays (SAM) identified 86 genes that distinguished EMC from the other sarcomas with 0.25% likelihood of false significance. NMB, DKK1, DNER, CLCN3, and DEF6 were the top five genes in this analysis. In situ hybridization for NMB gene expression on tissue microarrays (TMAs) containing a total of 1164 specimens representing 62 different sarcoma types and 15 different carcinoma types showed that NMB was highly expressed in 17 of 22 EMC cases and very rarely expressed in other tumours and thus could function as a novel diagnostic marker. High levels of expression of PPARG and the gene encoding its interacting protein, PPARGC1A, in most EMCs suggest activation of lipid metabolism pathways in this tumour. Small molecule inhibitors for PPARG exist and PPARG could be a potential therapeutic target for EMC.

Materials and Methods

Tumour samples. Ten cases of EMC were used for the gene expression studies. The clinical features of these ten cases are shown in Table 5. All cases examined had classical histology consistent with EMC and were reviewed by at least two pathologists with expertise in soft tissue tumors. The tissues were frozen and stored at −80° C. at the time of procurement. The institutional review board at Stanford University approved the study. For comparison, we used five cases each of gastrointestinal stromal tumour (GIST) and synovial sarcoma (SS); four cases each of leiomysarcoma (LMS) and malignant fibrous histiocytoma (MFH); and eight cases of dermatofibrosarcoma protuberans (DFSP). The sarcomas used for comparison purposes have been previously published.

| STT | | Age | | | Size |
|---|---|---|---|---|---|
| No | Diagnosis | (years) | Sex | Site | (cm) |
| 2528 | EMC | 51 | Male | Perineum | NA |
| 1169 | EMC | 66 | Male | Leg, left | 15 |
| 3783 | EMC | 54 | Female | Pelvic region, right | 9 |
| 3782 | EMC | 40 | Female | Thigh, left | 7.5 |
| 3780 | EMC | 61 | Female | Thigh, right | 14 |
| 3699 | EMC | 53 | Male | Thigh, right | 16 |
| 2003 | EMC | 69 | Male | Thigh, right | 8 |
| 3696 | EMC | 49 | Male | Groin | 12 |
| 3697 | EMC | 69 | Male | Thigh, right | 8 |
| 3698 | EMC | 71 | Male | Leg, right | 6 |

STT = soft tissue tumour,
EMC = extraskeletal myxoid chondrosarcoma;
NA = not available.

Gene expression using cDNA microarrays. The cDNA microarrays used in the study contained a total of 42 000 cDNA spots representing approximately 28 000 genes or expressed sequence tags (ESTs) printed on polylysine-coated glass slides by the Stanford Functional Genomics Facility. Preparation and details of microarray construction, isolation of mRNA from tumour tissues, labelling, and hybridization have been described previously. Briefly, tissue was homogenized in Trizol reagent (Invitrogen, Carlsbad, Calif., USA) and total RNA was extracted, followed by mRNA isolation using the FastTrack 2.0 method according to the manufacturer's protocol. Preparation of Cy3-dUTP (green fluorescent)-labelled cDNA from reference mRNA and Cy5-dUTP (red fluorescent)-labelled cDNA from 2 [symbol] g of each tumour specimen mRNA, microarray hybridization, and washing of arrays were performed as previously described. The reference mRNA was obtained from Stratagene (La Jolla, Calif., USA).

Microarrays were scanned on a GenePix 4000 microarray scanner (Axon Instruments, Foster City, Calif., USA) and fluorescence ratios (tumour/reference) were calculated using GenePix software. The raw data and the image files from these experiments are available from the Stanford Microarray Database and the filtered dataset is available through the accompanying website. Data were selected using the following criteria: control and empty spots on the arrays were not included in the analysis, as well as those spots manually flagged as not measurable. Only cDNA spots with a ratio of signal over background of at least 2.0 in either the Cy3 or the Cy5 channel were included. Genes with less than 80% well-measured data were not selected. A final filtering criterion was for genes whose expression level differed by at least four-fold in at least three arrays. Using these criteria, 2918 genes passed the filtering criteria and were used for further analysis. Unsupervised hierarchical clustering analysis and significance analysis of microarrays (SAM) were then performed as described previously.

Tissue microarray (TMA) construction. A TMA of 464 soft tissue tumours (TA-38, TA-39) was constructed using a manual tissue arrayer (Beecher Instruments, Silver Spring, Md., USA) following previously described techniques. Duplicate 600 [symbol] m cores were taken from paraffin-embedded soft tissue tumour samples archived at the Stanford University Medical Center, Department of Pathology between 1995 and 2001. The cores were taken from areas in the paraffin block that were representative of the diagnosis. Fifty-four different soft tissue tumour diagnostic entities were represented on TA-38 and TA-39. These tissue arrays are identical to TA-34 and TA-35 which are described in detail in a previous study. Furthermore, two new TMAs were generated that contained 19 cases of EMC, 24 cases of myxoid liposarcomas, and 25 cases of other sarcomas (TA-109) and TA-140 that contained 19 cases of pleomorphic adenomas. A total of 57 different sarcoma types were represented on TMAs TA-38/-39, TA-109, and TA-140. We also used a TMA (TA-03/008) [24] that contained a total of 121 cases in duplicate, including 62 chondrosarcomas, five EMCs, four each of chondromyxoid fibromas and chondroblastomas, and 30 enchondromas, ten osteosarcomas, and six osteochondromas. This array added an additional five soft tissue tumour (STT) types to the 57 represented on TA-38/TA-39 and TA-109/TA-140, for a total of 62 STT diagnostic entities. In addition, we used carcinoma TMAs (TA-41 and TA-42) containing 526 cases representing 15 different carcinomas, including colon, lung, prostrate, ovary, etc. The institutional review board at Stanford University approved the construction of these TMAs.

In situ hybridization (ISH). ISH of TMA sections was performed as previously described. Briefly, sense and anti-sense RNA probes were generated for NMB, PHLDA1, LRP5, and KIT by polymerase chain reaction amplification with the T7 promoter sequence added to the 5′ end of either forward or reverse primer to generate sense or anti-sense probes. In vitro transcription was performed with a digoxigenin RNA-labelling kit and T7 polymerase according to the manufacturer's protocol (Roche Diagnostics, Indianapolis, Ind., USA). Sections (4 μm thick) cut from the TMA blocks were dewaxed in xylene and hydrated in graded concentrations of ethanol for 5 min each. Sections were then incubated with 1% hydrogen peroxide, followed by digestion in 10 μg/ml proteinase K at 37° C. for 30 min. Sections were hybridized overnight at 55° C. with either sense or anti-sense riboprobes at 150 ng/ml dilution in mRNA hybridization buffer (DAKO). The following day, sections were washed in 2× saline sodium citrate (SSC) and incubated with a 1:35 dilution of RNase A cocktail (Ambion, Austin, Tex., USA) in 2×SSC for 30 min at 37° C. Next, sections were stringently washed in 2×SSC-50% formamide twice, followed by one wash in 0.08×SSC at 55° C. Biotin-blocking reagents (DAKO) were applied to the section to block endogenous biotin. For signal amplification, a horseradish peroxidase conjugated rabbit anti-digoxigenin antibody (DAKO) was used to catalyze the deposition of biotinyl tyramide, followed by secondary streptavidin-horseradish peroxidase complex (GenPoint kit, DAKO). The final signal was developed with diaminobenzidine (Gen-Point kit, DAKO) and the tissues were counterstained in haematoxylin for 15 s.

TABLE 2
The first 50 genes with the highest differential expression in EMC
according to SAM analysis. Input parameters for SAM analysis:
imputation engine is 10 nearest neighbour imputer; data
type is two class-unpaired data; data are in log scale; number of
permutations is 100; RNG seed is 1234567; delta fold-changes
are 1.86835: and median number of false significant is 0.2205

| Gene | | | Score | |
|---|---|---|---|---|
| symbol | Description | Unigene ID | (d) | Fold-change |
| NMB | Neuromedin B | Hs.386470 | 6.560 | 20.28606 |
| DKK1 | Dickkopf homologue 1 (Xenopus laevis) | Hs.40499 | 6.165 | 65.64312 |
| DNER | Delta-notch-like EGF repeat-containing transmembrane | Hs.234074 | 5.016 | 14.68225 |
| CLCN3 | Chloride channel 3 | Hs.372528 | 5.010 | 6.79669 |
| DEF6 | Differentially expressed in FDCP 6 homologue (mouse) | Hs.15476 | 4.804 | 8.29296 |
| RNF130 | Ring finger protein 130 | Hs.155718 | 4.803 | 5.02955 |
| C10orf116 | chromosome 10 open reading frame 116 | Hs.511763 | 4.682 | 10.14755 |
| ADORA2A | Adenosine A2a receptor | Hs.197029 | 4.657 | 5.44355 |
| CTNND2 | Catenin (cadherin-associated protein), delta 2 | Hs.436421 | 4.499 | 9.68996 |
| PAM | Peptidylglycine alpha-amidating monooxygenase | Hs.352733 | 4.420 | 7.30293 |
| GCLC | Glutamate-cysteine ligase, catalytic subunit | Hs.414985 | 4.395 | 10.35690 |
| CPD | Carboxypeptidase D | Hs.5057 | 4.361 | 8.13674 |
| FADS2 | Fatty acid desaturase 2 | Hs.388164 | 4.332 | 6.09523 |
| HIP1R | Huntingtin interacting protein-1-related | Hs.96731 | 4.279 | 10.00849 |
| GARNL4 | GTPase activating RANGAP domain-like 4 | Hs.499659 | 4.257 | 6.24861 |
| LRRN1 | Leucine-rich repeat neuronal 1 | Hs.126085 | 4.215 | 6.04359 |
| PGAM2 | Phosphoglycerate mutase 2 (muscle) | Hs.413238 | 4.190 | 6.68866 |
| LRP5 | Low density lipoprotein receptor-related protein 5 | Hs.6347 | 4.169 | 5.04694 |
| BACH | Brain acyl-CoA hydrolase | Hs.435092 | 4.119 | 4.74086 |
| MAPK12 | Mitogen-activated protein kinase 12 | Hs.432642 | 3.942 | 4.19472 |
| SNCA | Synuclein, alpha (non-A4 component of amyloid precursor) | Hs.76930 | 3.913 | 16.42230 |
| IRS2 | Insulin receptor substrate 2 | Hs.143648 | 3.909 | 5.20002 |
| CDK7 | Cyclin-dependent kinase 7 | Hs.184298 | 3.895 | 14.36505 |
| MAN1A1 | Mannosidase, alpha, class 1A, member 1 | Hs.255149 | 3.877 | 14.92724 |
| DOC-1R | Tumour suppressor deleted in oral cancer-related 1 | Hs.379039 | 3.875 | 6.15741 |
| PHLDA1 | Pleckstrin homology-like domain, family A, member 1 | Hs.82101 | 3.834 | 6.58961 |
| CRIP2 | Cysteine-rich protein 2 | Hs.70327 | 3.765 | 4.33354 |
| NDRG2 | NDRG family member 2 | Hs.243960 | 3.749 | 4.25865 |
| SOD3 | Superoxide dismutase 3, extracellular | Hs.2420 | 3.746 | 6.75870 |
| HSPB8 | Heat shock 22 kD protein 8 | Hs.111676 | 3.725 | 5.55067 |
| NPDC1 | Neural proliferation, differentiation and control, 1 | Hs.105547 | 3.721 | 3.83682 |
| PCTK3 | PCTAIRE protein kinase 3 | Hs.445402 | 3.709 | 5.83566 |
| DPP6 | Dipeptidylpeptidase 6 | Hs.390175 | 3.698 | 4.13856 |
| MYC | v-myc myelocytomatosis viral oncogene homologue (avian) | Hs.202453 | 3.669 | 3.79501 |
| FOXC1 | Forkhead box C1 | Hs.348883 | 3.646 | 6.39417 |
| ZNF92 | Zinc finger protein 92 (HTF12) | Hs.9521 | 3.637 | 3.80734 |
| RBAF600 | Retinoblastoma-associated factor 600 | Hs.287616 | 3.577 | 3.84612 |
| PPP1R3C | Protein phosphatase 1, regulatory (inhibitor) subunit 3C | Hs.303090 | 3.562 | 5.56192 |
| LMO4 | UM domain only 4 | Hs.3844 | 3.553 | 3.93953 |
| CSPG2 | Chondroitin sulphate proteoglycan 2 (versican) | Hs.434488 | 3.519 | 7.28297 |
| POLR3G | Polymerase (RNA) III (DNA directed) polypeptide G | Hs.282387 | 3.511 | 4.57622 |
| FOXC1 | Forkhead box C1 | Hs.348883 | 3.646 | 3.62360 |
| NRN1 | Neuritin 1 | Hs.103291 | 3.446 | 7.49085 |
| NRBP | Nuclear receptor binding protein | Hs.272736 | 3.446 | 5.96659 |
| PPARG | Peroxisome proliferative activated receptor, gamma | Hs.387667 | 3.413 | 4.26804 |
| PTPRM | Protein tyrosine phosphatase, receptor type, M | Hs.154151 | 3.412 | 4.03699 |
| TUBB1 | Tubulin. beta 1 | Hs.303023 | 3.407 | 5.37414 |
| SEC6L1 | SEC6-like 1 (S. cerevisiae) | Hs.448580 | 3.404 | 7.22878 |
| MMP24 | Matrix metalloproteinase 24 (membrane-inserted) | Hs.212581 | 3.396 | 3.68382 |
| MAPT | Microtubule-associated protein tau | Hs.101174 | 3.363 | 5.94829 |

TABLE 3
Staining results for NMB in situ hybridization on TMAs
TA-38/-39, TA-109, TA-140, and TA-03/008 in tabular form.
The number of positives includes cases that stained either
strong or weakly. Only sarcomas represented by at least nine
different cases on the tissue array are given in this table. The
staining results on sarcoma types for which less than nine cases
were present are shown in Supplementary Table 6

| | No | No positive | % positive |
|---|---|---|---|
| Diagnosis | of cases | for NMB | for NMB |
| Angiosarcoma | 13 | 3 | 23.1 |
| Chondrosarcoma | 64 | 0 | 0.0 |
| DSRCT | 14 | 0 | 0.0 |
| DFSP | 9 | 1 | 11.1 |
| Enchondroma | 29 | 0 | 0.0 |
| EMC | 22 | 18 | 81.8 |
| Endometrial stromal sarcoma | 12 | 0 | 0.0 |
| Epithelioid sarcoma | 12 | 0 | 0.0 |
| Fibromatosis | 17 | 2 | 11.8 |
| GIST | 29 | 4 | 13.8 |
| Haemangioendothelioma | 10 | 0 | 0.0 |
| Leiomyoma | 10 | 2 | 20.0 |
| Leiomyosarcoma | 40 | 1 | 2.5 |
| Lipomatous tumour | 39 | 1 | 2.6 |
| MFH | 61 | 6 | 9.8 |
| Myxoid liposarcoma | 24 | 0 | 0.0 |
| Osteosarcoma | 10 | 0 | 0.0 |
| Pleomorphic adenomas | 19 | 0 | 0.0 |
| Rhabdomyosarcoma | 17 | 0 | 0.0 |
| SFT | 13 | 1 | 7.7 |
| Synovial sarcoma | 21 | 0 | 0.0 |
| Tenosynovial giant cell tumour | 9 | 0 | 0.0 |

Immunohistochemistry. Anti-KIT antiserum (rabbit polyclonal, 1:50; DAKO) was used on 4 [symbol] m sections from the tissue array blocks that were dewaxed in xylene, and hydrated in a graded series of alcohol. Staining was then performed using the EnVision+ anti-rabbit system (DAKO).

Scoring of immunohistochemistry and ISH. Cores were scored as follows: a score of −2 was given for negative staining, defined as fewer than 5% of tumour cells showing staining at or minimally above background. A score of 1 (weak positive staining) was given for light brown staining in greater than 5% of tumour cells. A score of 2 (strong positive staining) was given for dark brown staining in greater than 50% of tumour cells. Non-tumour cells and cells of unknown origin were not scored. Two pathologists (MvdR and RW) independently scored the stains and disagreements were reviewed together to achieve a consensus score. Scoring results were combined using Deconvoluter and Compressor programmes and represented as a clustered dataset in Treeview.

DNA sequencing. KIT gene sequencing was carried out using a combination of denaturing HPLC and direct sequencing, as previously described.

Results

Gene expression analysis. We analyzed the gene expression profiles for ten cases of EMC with 42 000 spot cDNA microarrays and compared them with 26 previously reported soft tissue tumours. The clinical features for the ten EMC cases are shown in Table 5. After passing the predetermined filtering criteria of (1) a ratio of 2.0 mean florescence intensity versus background intensity for each spot in either Cy3 or Cy5 channels and (2) an absolute value of greater than four-fold expression, relative to the mean expression across all 36 cases, in at least three samples, 8862 spots remained from the initial dataset. A further selection for genes that had at least 80% measurable data (ie measurable results in at least 28 tumours) left 2918 genes that passed all the filtering criteria. The 2918 genes and 36 tumour samples were grouped using unsupervised hierarchical clustering, which is an analysis that clusters the genes into groups with similar expression patterns across the tumours tested and clusters the tumour specimens based on their gene expression profile. All ten cases of EMC clustered together, indicating that they were closely related to each other and significantly different from the other tumours profiled. The EMC specimens were distinguished from other neoplasms by a large cluster of about 560 highly expressed genes.

Marker Discovery.

Significance analysis of microarrays (SAM). We analysed the expression data by SAM to identify and rank order the genes that differentiate EMCs from other sarcomas. The EMC cases were also very distinct from other sarcomas by this analysis. Eighty-six genes distinguished this tumour from the other sarcomas with 0.25% probability of false significance. NMB was the top-ranking gene in SAM and was highly expressed in all EMC samples (FIG. 1B). The top 50 genes identified by SAM are shown in Table 6.

Neuromedin B (NMB) is a specific marker for EMC. In order to validate potential new diagnostic markers for EMC identified through the gene expression analyses, we generated ISH probes against three genes: NMB, PHLDA1, and LRP5. These genes were chosen based on the gene ranking in the SAM and red channel (Cy5) intensity, a measure of the absolute amount of RNA in the sample. For ISH testing, we used a previously described sarcoma TMA, TA-38FrA-39, consisting of 986 cores (464 cases) and two novel TMAs, TA-109 and TA-140, that included duplicate cores from three gastrointestinal stromal tumours (GISTs), 19 EMCs, 24 myxoid liposarcomas (MLSs), ten desmoplastic small round cell tumours (DSRCTs), and 19 pleomorphic adenomas. Combined, these arrays represent 57 different sarcoma types. The sense strand probes for NMB, PHLDA1, and LRP5 served as negative controls.

Strong staining for NMB was seen for 15 of 19 scoreable EMC cases; one of the remaining four cases was weakly positive. Strong NMB staining was predominantly confined to EMCs and only one of the non-EMC cases, an MFH, was strongly positive. A small number of other sarcomas showed weak staining for NMB, including 5/61 MFH and 1/40 LMS cases (Table 8). PHLDA1 showed high levels of expression by gene array studies in nine of ten EMCs but was also weakly expressed in GISTs. By ISH, PHLDA1 stained 12 of 19 (63.1%) of the scoreable cases of EMC. PHLDA1 was weakly positive in 4 of 28 (14.2%) of the scoreable GIST cases. In gene arrays, LRP5 showed high levels of expression in nine of ten EMCs; LRP5 was also weakly expressed in two of the synovial sarcomas included in the gene array studies. On TMAs, LRP5 stained 12 of 19 (63.1%) cases of EMC by ISH. However, a significant number of other sarcomas demonstrated at least weak staining for LRP5.

With NMB showing the highest degree of specificity, we used a TMA (TA-03/008) that contained a wide variety of cartilaginous lesions to evaluate the specificity of NMB ISH. The tissue array TA-03/008 contained five new diagnostic entities and a total of 121 cases in duplicate, including 62 chondrosarcomas, five EMCs, four each of chondromyxoid fibromas and chondroblastomas, and 30 enchondromas, ten osteosarcomas, and six osteochondromas. Of the five EMC cases on this tissue array, two were strongly positive, two were unscoreable due to lack of tissue, and one was negative for NMB staining, while none of the other sarcomas on the tissue array were positive for NMB. Considering all TMAs, NMB showed strong expression in 17 of 22 EMC cases available and weak expression in one EMC case. To evaluate further the specificity of NMB ISH, we extended our observations to a carcinoma TMA (TA-41/TA-42) that contained 526 cores representing carcinomas from many different primary sites. Of the 438 scoreable cores on the tissue array, none showed strong staining and only seven showed weak staining for NMB. These included renal cell carcinoma (1/36), transitional cell carcinoma of the bladder (1/25), squamous cell carcinoma of the lung (2/16), and thyroid papillary carcinoma (3/18).

Gene expression modules in EMC. Signalling pathway-related genes Several different signalling pathways are represented within the EMC gene cluster of highly expressed genes. Signal transduction genes involved in adipocytic differentiation are identified. The genes CITED2, CPT1B, and PPARGC1A act as co-activators in the pathway mediated by the peroxisome proliferator, PPAR-alpha. Peroxisome proliferators regulate gene expression by forming a heterodimeric complex with PPAR/RXR and binding to a peroxisome proliferator-response element (PPRE). Peroxisome proliferators are involved in lipid metabolism. Although PPAR-alpha itself is not expressed in EMCs, another gene that belongs to the PPAR family, PPARG, was significantly expressed in most of the EMCs. PPARG is a key regulator of adipocyte differentiation and glucose homeostasis. PPARGC1A, which is a peroxisome proliferator co-activator and also an interacting protein with PPARG, that allows the interaction of PPARG with multiple transcription factors involved in a wide variety of pathways, was also strongly expressed. DKK1 (a Wnt antagonist) and LRP5 (a Wnt coreceptor), genes involved in the Wnt signalling pathway, are highly expressed in most cases of EMC. Expression of DKK1 was shown to promote growth and expansion of mesenchymal cells: this suggests that DKK1 may induce growth and tumourigenesis in EMCs. LRP5 has been implicated in disease progression in high-grade osteosarcoma.

Other genes highly expressed in EMC that are involved in signalling pathways include PTPRM, PCTK3, MAPK12, JUN, MYC, and CLCN3. The majority of EMCs express both CSPG2 (chondroitin sulphate proteoglycan 2) and MMP24 (matrix metalloproteinase 24). CSPG2 is a protein that may play a role in intercellular signalling and in connecting cells with the extracellular matrix. MMP24 is involved in degradation of proteoglycans, such as dermatan sulphate and chondroitin sulphate proteoglycan, of which CSPG2 is a member. The stroma of EMC consists predominantly of chondroitin-4 and 6-sulphate and keratan sulphate. An isoform of chondroitin sulphate, proteoglycan V1, plays a major role in neuronal differentiation and neurite outgrowth.

KIT gene expression in EMC. In our gene expression analysis, high levels of KIT expression were noted in six of the ten EMCs. Among these six cases, four showed KIT expression at levels comparable to that seen in GIST. We confirmed KIT expression in a subset of EMCs with a separate set of EMC cases on TMAs using ISH and immunohistochemistry (IHC). With IHC, 8 of 19 scoreable cases (42.1%) were positive for KIT. By ISH, 6 of 11 scoreable cases (54.5%) were positive for KIT. These findings suggest a possible mutation in KIT, as described for GIST. We subsequently screened exons 9, 11, 13, and 17 of the KIT gene from these six cases of EMC but did not identify any mutations. Furthermore, no consistent differences were seen in gene expression between KIT-positive and KIT-negative EMCs, and KIT-positive EMC did not share expression of other genes with GISTs.

Evidence for neural-neuroendocrine differentiation. Many genes that suggest neuroendocrine differentiation were expressed in EMC. EN02 and INSM1 are considered to be markers for neuroendocrine differentiation and were detected in seven of ten EMC cases. SYP, CHGA, NEF3, and GAD2 are also thought to be neuroendocrine differentiation markers and each was expressed in at least three of the ten cases of EMC. These genes did not meet the gene-filtering criteria used in these experiments. Nevertheless, these four genes do show increased expression in EMC compared with other sarcomas. We also noticed increased expression of NPDC1 and NDRG2, which are implicated in neuroendocrine differentiation, in EMC. A number of genes that are associated with different neuronal functions are expressed in EMC, including CLCN3, PHLDA1, CTNVD2, NRN1, OLFM1, PAM, LRRN1, CELSR2, SYNJ, DNER, and BGN. In addition to genes that have a neuronal function, two genes (SNCA and SNGG) that play a role in neurodegenerative disease are expressed in EMC.

Synuclein-alpha (SNCA) and synucleingamma (SNCG) are members of the synuclein family of proteins, which are believed to be involved in the pathogenesis of neurodegenerative diseases. SNCA induces fibrillization of microtubule-associated protein tau. High levels of SNCG have been identified in advanced breast carcinomas, suggesting a correlation between overexpression of SNCG and tumour development.

Cell proliferation genes in EMC. Various cell proliferation and cell migration genes are present in the EMC gene cluster. Overexpression of IRS2 (insulin receptor substrate 2) in EMC may enhance mitogenic signalling. Furthermore, high levels of expression of connective tissue growth factor (CTGF) in a subset of EMCs suggest that CTGF may promote proliferation. Three other genes (TM4SF2, CDK7, and ERK8) that are involved in regulation of cell proliferation are also highly expressed in EMCs.

Microtubules in EMC. Ultrastructural studies have shown the presence of densely packed bundles or parallel arrays of microtubules in EMC. In our studies, we noticed high levels of expression of microtubule-associated genes such as MAP7, TUBB1, TUBB5, and MAPT. Of these genes, MAPT (microtubule-associated protein tau) is differentially expressed in the nervous system, depending on the stage of neuronal maturation and neuron type.

Unsupervised hierarchical clustering of ten EMCs and a total of 26 other sarcomas used in the study revealed that the EMCs were closely related based on their gene expression profiles. Two class unpaired SAM on the final gene set selected after gene filtering revealed many genes that are significantly associated with EMC. NMB, DKK1, DNER, CLCN3, and DEF6 were the top five genes that distinguished EMC from the other sarcomas.

EMC is one of several soft tissue tumours that has a fusion protein involving the EWS gene. The fusion proteins containing EWS have been shown to possess strong transcriptional regulatory activity. The presence of a DNA-binding domain in the EWS fusion proteins suggests that the fusion protein may exert its oncogenic potential by deregulating the expression of specific target genes. We searched our gene expression data for known downstream genes affected by fusion proteins in other sarcomas. We identified three genes. (ID2, MYC, and TM4SF2) that were highly expressed in EMC and that are known to be affected in tumours with other EWS fusion proteins. The high expression of these three downstream targets (ID2, MYC, and TM4SF2) in the majority of the EMCs suggests that some of the fusion partners associated with EWS may have common gene targets in the different sarcomas where EWS is used as a partner in translocation.

A significant number of genes were differentially expressed in EMC. One of these is Neuromedin B (NMB), a mammalian homologue of amphibian bombesin and a secreted neuropeptide involved in stimulation of smooth muscle contraction. NMB is a potent mitogen and growth factor for normal and neoplastic lung and for gastrointestinal epithelial tissue. NMB was among the first neuropeptides to be implicated as an autocrine growth factor in lung cancer cells. In situ hybridization using NMB on our TMAs containing a total of 1164 specimens of 62 different types of soft tissue tumour and 15 types of carcinoma showed that NMB was highly expressed in EMCs (17 of 22) but rarely in other tumours. The predominant expression of NMB in EMC indicates that it may be useful in the diagnosis of EMC.

Myoepithelial/mixed tumours and myxoid liposarcomas are regarded as the main differential diagnosis of EMC. NMB was negative in 19 pleomorphic adenomas of the salivary gland and 24 myxoid liposarcomas. NMB is a secreted protein. If it is found in higher levels in the serum of patients, it may serve as a useful serum marker in the diagnosis of recurrence of EMC. This could be of clinical interest as EMC can recur, sometimes after very long periods of time. Patients with EMC currently have to undergo repeated imaging studies for follow-up.

In our study, expression of genes such as ENO2, SYP, CHGA, NEF3, GAD2, and INSM1 in EMC suggests neural-neuroendocrine differentiation. Expression of genes involved in pathways mediated by peroxisome proliferators in EMC suggests that lipid/fat metabolism may be affected in this tumour. PPARs play a critical physiological role as lipid sensors and regulators of lipid metabolism, being activated by fatty acids and eicosanoids. PPARG is highly expressed in most cases of EMC. PPARG regulates fatty acid catabolism, and is involved in inflammation and in the cell response to reactive oxygen species. PPARG promotes lipogenesis and exerts anti-inflammatory and anti-proliferative actions. Our finding of high levels of PPARG could have clinical implications, as PPARG has been shown to be a potential therapeutic target. The PPARG antagonist, GW9662 (2-chloro-5-nitro-N-phenylbenzamide), is cell-permeable, selective, and irreversible. Other small molecule inhibitors of PPARG are Oarylmandelic acid and BADGE [2,2-bis(4_-glycidyloxyphenyl)propane]. Furthermore, TZD18, a novel PPARalpha/gamma dual agonist, has been shown to inhibit cell growth and induce apoptosis in human glioblastoma T98G cells in vitro, indicating a therapeutic potential for this compound.

In summary, using global gene expression profiling, we have identified a gene expression signature of EMC. We found evidence for neuralneuroendocrine differentiation in a majority of EMCs and noticed a significant number of genes that are associated with neuronal function. EMCs show increased expression of several genes that are up-regulated by other fusion proteins that involve EWS as one of the translocation partners. This observation suggests that fusion proteins involved in different sarcomas could have common transcriptional targets. The integrated approach of using gene expression analysis and tissue microarrays resulted in the discovery of the potential diagnostic marker, neuromedin B, for EMC. As it is a secreted protein, neuromedin B may prove to be a serological marker of EMC recurrence. High levels of expression of PPARG and the gene encoding its interacting protein, PPARGC1A, suggest that lipid metabolism is affected in this tumour. The availability of selective small molecule inhibitors for PPARG raises the possibility of using PPARG as a therapeutic target in treating patients with EMC.

What is claimed is:

1. A method for classification of a solid tumor other than a soft tissue tumor and comprising a stromal cell component, the method comprising:
comparing expression of genetic sequences in a sample of said solid tumor with a soft tissue gene expression set (STS);
and classifying said solid tumor according to its relationship with said STS.

2. The method according to claim 1, wherein said solid tumor is a carcinoma.

3. The method according to claim 2, wherein said expression of genetic sequences is determined by microarray hybridization.

4. The method according to claim 3, the method comprising:
extracting mRNA from said carcinoma;
quantitating the level of mRNA corresponding to STS sequences;
comparing said level of mRNA to the level of said mRNA in a reference sample.

5. The method according to claim 4, wherein said comparing step comprises determination of statistical correlation.

6. The method according to claim 2, wherein said expression of genetic sequences is determined by in situ hybridization.

7. The method according to claim 1, wherein said STS is derived from at least one of Evan's tumor; nodular fasciitis; desmoid-type fibromatosis; solitary fibrous tumor; dermatofibrosarcoma protuberans (DFSP); angiosarcoma; epithelioid hemangioendothelioma; tenosynovial giant cell tumor (TGCT); pigmented villonodular synovitis (PVNS); fibrous dysplasia; myxofibrosarcoma; fibrosarcoma; synovial sarcoma; malignant peripheral nerve sheath tumor; neurofibroma; and pleomorphic adenoma of soft tissue.

8. The method according to claim 1, wherein said STS comprises information from at least about 20 genes.

9. The method according to claim 8, wherein said STS is derived by the method comprising:
hybridizing mRNA from at least one particular soft tissue tumor to obtain a set of hybridization data;
filtering said hybridization data to select for sequences having a pre-determined ratio of hybridization intensity to background intensity;
filtering said hybridization data to select for sequences having an absolute level of expression relative to the mean expression level within the tumor classification;
filtering said hybridization data to select for sequences having at least about 70% measurable data in said sample;
grouping filtered data by unsupervised hierarchical clustering across data from unrelated soft tissue tumors and selecting a set of genes that distinguish the soft tissue tumor from other soft tissue tumors.

10. A method of obtaining a genetic signature useful in the classification of a stromal component of a carcinoma, the method comprising:
hybridizing mRNA from at least one particular soft tissue tumor to obtain a set of hybridization data;
filtering said hybridization data to select for sequences having a pre-determined ratio of hybridization intensity to background intensity;
filtering said hybridization data to select for sequences having an absolute level of expression relative to the mean expression level within the tumor classification;
filtering said hybridization data to select for sequences having at least about 70% measurable data in said sample;
grouping filtered data by unsupervised hierarchical clustering across data from unrelated soft tissue tumors and selecting a set of genes that distinguish the soft tissue tumor from other soft tissue tumors.

11. Use of the genetic signature of claim 10 as a probe for in situ hybridization to a carcinoma.

12. Use of the genetic signature of claim 10 as a platform for target discover of polypeptides useful as targets in treatment of a carcinoma.

13. A kit for cancer classification, the kit comprising:
a set of primers specific for at least 25 STS genes; and instructions for use.

14. The kit according to claim 13, further comprising a software package for statistical analysis of expression profiles, and a reference dataset for a STS signature.
